(19) United States
(12) Patent Application Publication
(10) Pub. No.: US 2010/0029697 A1
(43) Pub. Date: Feb. 4, 2010

(54) SUBSTITUTED PYRIDO[3,2-E][1,2,4]TRIAZOLO[4,3-C]PYRIMIDINE DERIVATIVES AS CANNABINOID-1 RECEPTOR MODULATORS

(76) Inventors: John S. Debenham, Scotch Plains, NJ (US); Christina B. Madsen-Duggan, Scotch Plains, NJ (US); Jeffrey J. Hale, Westfield, NJ (US); Thomas F. Walsh, Watchung, NJ (US)

Correspondence Address: MERCK AND CO., INC, P O BOX 2000, RAHWAY, NJ, 07065-0907, US

(21) Appl. No.: 12/529,889

(22) PCT Filed: Mar. 24, 2008
(86) PCT No.: PCT/US08/03847

§ 371 Date: Sep. 3, 2009

Related U.S. Application Data
(60) Provisional application No. 60/920,392, filed on Mar. 28, 2007.

(51) Int. Cl. A61K 31/519; C07D 471/14; A61P 3/04; A61P 25/00; A61P 25/28; A61P 25/16; A61P 25/06; A61P 1/16; A61P 11/06; A61P 25/30

(52) U.S. Cl. 514/267; 544/251

(57) ABSTRACT

Novel compounds of the structural formula (I) are antagonists and/or inverse agonists of the Cannabinoid-1 (CB1) receptor and are useful in the treatment, prevention and suppression of diseases mediated by the CB1 receptor. The compounds of the present invention are useful as centrally acting drugs in the treatment of psychosis, memory deficits, cognitive disorders, Alzheimer s disease, migraine, neuropathy, neuro-inflammatory disorders including multiple sclerosis and Guillain-Barre syndrome and the inflammatory sequelae of viral encephalitis, cerebral vascular accidents, and head trauma, anxiety disorders, stress, epilepsy, Parkinson s disease, movement disorders, and schizophrenia. The compounds are also useful for the treatment of substance abuse disorders, the treatment of obesity or eating disorders, as well as the treatment of asthma, constipation, chronic intestinal pseudo-obstruction, cirrhosis of the liver, non-alcoholic fatty liver disease (NAFLD), non-alcoholic steatohepatitis (NASH), and the promotion of wakefulness.

### BACKGROUND OF THE INVENTION

Marijuana (Cannabis sativa L.) and its derivatives have been used for centuries for medicinal and recreational purposes. A major active ingredient in marijuana and hashish has been determined to be Δ⁹-tetrahydrocannabinol (Δ⁹-THC). Detailed research has revealed that the biological action of Δ⁹-THC and other members of the cannabinoid family occurs through two G-protein coupled receptors termed CB1 and CB2. The CB1 receptor is primarily found in the central and peripheral nervous systems and to a lesser extent in several peripheral organs. The CB2 receptor is found primarily in lymphoid tissues and cells. Three endogenous ligands for the cannabinoid receptors derived from arachidonic acid have been identified (anandamide, 2-arachidonoyl glycerol, and 2-arachidonyl glycerol ether). Each is an agonist with activities similar to Δ⁹-THC, including sedation, hypothermia, intestinal immobility, antinociception, analgesia, catalepsy, anti-emesis, and appetite stimulation.

There are at least three CB1 modulators characterized as inverse agonists/antagonists, ACOMPLIA (rimonabant, N-(1-piperidinyl)-5-(4-chlorophenyl)-1-(2,4-dichlorophenyl)-4-methylpyrazole-3-carboxamide, SR141716A), and 3-(4-chlorophenyl-N′-(4-chlorophenyl)sulfonyl-N-methyl-4-phenyl-4,5-dihydro-1H-pyrazole-1-carboxamide (SLV-319), and taranabant, N-[(1S,2S)-3-(4-Chlorophenyl)-2-(3-cyanophenyl)-1-methylpropyl]-2-methyl-2-[[5-(trifluoromethyl)-2-pyridinyl]oxy]propanamide, in clinical development for treatment of eating disorders and/or smoking cessation at this time. There still remains a need for potent low molecular weight CB1 modulators that have pharmacokinetic and pharmacodynamic properties suitable for use as human pharmaceuticals.

Naphthyridone CB1 antagonists/inverse agonists are described in Debenham, et al., Bioorg. Med. Chem. Lett. 16: 681-685 (2006) and in WO 05/047285. Substituted naphthyridine AKT inhibitors are disclosed in WO 2006/135627. Aryl[e]fused pyrazolo[4,3-c]pyridines are described in Forbes et. al, J. Med. Chem. 33, 2640-2645 (1990). Novel fused Triazolone derivatives are described in WO 2004/081008. Quinazolines are disclosed in Jantova et al., Biologia (Bratislava, Slovakia) (2004), 59(6), 741-752, and in WO 95/32205.

### SUMMARY OF THE INVENTION

The present invention is concerned with novel pyrido triazolo pyrimidines of structural Formula I:

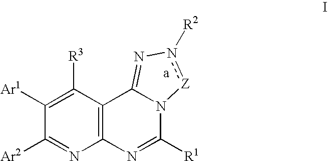

and pharmaceutically acceptable salts thereof which are modulators of and, in particular, antagonists and/or inverse agonists of the Cannabinoid-1 (CB1) receptor and are useful in the treatment, prevention or suppression of diseases mediated by the Cannabinoid-1 (CB1) receptor. In one aspect, the invention is concerned with the use of these novel compounds to selectively antagonize the Cannabinoid-1 (CB1) receptor. As such, compounds of the present invention are useful as centrally acting drugs in the treatment of psychosis, memory deficits, cognitive disorders, Alzheimer's disease, migraine, neuropathy, neuro-inflammatory disorders including multiple sclerosis and Guillain-Barre syndrome and the inflammatory sequelae of viral encephalitis, cerebral vascular accidents, and head trauma, anxiety disorders, stress, epilepsy, Parkinson's disease, movement disorders, and schizophrenia. The compounds are also useful for the treatment of substance abuse disorders, the treatment of obesity or eating disorders, and complications associated therewith, including left ventricular hypertrophy, as well as the treatment of asthma, constipation, chronic intestinal pseudo-obstruction, and cirrhosis of the liver.

The present invention is also concerned with treatment of these conditions, and the use of compounds of the present invention for manufacture of a medicament useful in treating these conditions. The present invention is also concerned with treatment of these conditions through a combination of compounds of formula I and other currently available pharmaceuticals.

The invention is also concerned with pharmaceutical formulations comprising one of the compounds as an active ingredient, as well as processes for preparing the compounds of this invention.

### DETAILED DESCRIPTION OF THE INVENTION

The compounds of the present invention are represented by the compound of structural formula I:

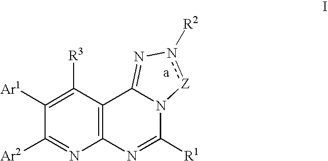

or a pharmaceutically acceptable salt thereof, wherein:
Z is selected from:

(1) C═O, and

(2) CR⁸;

“a” is:

(1) a single bond when R² is present and Z is C═O, or

(2) a double bond when R² is absent and Z is CR⁸;

Ar¹ is selected from:

(1) aryl, and

(2) heteroaryl,

wherein aryl and heteroaryl are unsubstituted or substituted with one, two, three or four substituents selected from R⁴ and R⁵;
Ar² is aryl, wherein aryl is unsubstituted or substituted with one, two, three or four substituents independently selected from R⁶ and R⁷;
R¹ is selected from:

(1) C₁₋₁₀ alkyl,

(2) C₃₋₁₀cycloalkyl,

(3) C₃₋₁₀cycloalkenyl,

(4) C₃₋₁₀cycloalkyl-C₁₋₄alkyl,

(5) C₃₋₁₀cycloalkenyl-C₁₋₄alkyl,

(6) cycloheteroalkyl,

(7) cycloheteroalkyl-C₁₋₄alkyl,

(8) aryl,

(9) aryl-C₁₋₄alkyl,

(10) heteroaryl,

(11) heteroaryl-C₁₋₄alkyl,

(12) —OR^{e}, and

(13) —NR^{c}R^{d},

wherein each alkyl is unsubstituted or substituted with one to four substituents independently selected from R^{a}, and each cycloalkyl, cycloalkenyl, cycloheteroalkyl, aryl and heteroaryl is unsubstituted or substituted with one to four substituents independently selected from R^{b};
R² is absent or selected from:

(1) hydrogen, and

(2) C₁₋₁₀ alkyl;

R³ is hydrogen;
each R⁴, R⁵, R⁶, and R⁷ is independently selected from:
(1) -hydrogen,
(2) -halogen,
(3) —CN,
(4) —C₁₋₆alkyl, unsubstituted or substituted with one, two or three R^{f} substitutents,
(5) —CF₃,
(6) —C₂₋₆alkenyl, unsubstituted or substituted with one, two or three R^{f} substitutents,
(7) -cycloalkyl, unsubstituted or substituted with one, two or three R^{f} substitutents,
(8) -cycloalkyl-C₁₋₃alkyl-, unsubstituted or substituted with one, two or three R^{f} substitutents,
(9) -cycloheteroalkyl, unsubstituted or substituted with one, two or three R^{f} substitutents,
(10) -aryl, unsubstituted or substituted with one, two or three R^{h} substitutents,
(11) -aryl-C₁₋₃alkyl-, unsubstituted or substituted on aryl with one, two or three R^{h} substitutents,
(12) -heteroaryl, unsubstituted or substituted with one, two or three R^{h} substitutents,
(13) -heteroaryl-C₁₋₃alkyl-, unsubstituted or substituted with one, two or three R^{h} substitutents,
(14) —OR^{d},
(15) —OCF₃,
(16) —C(O)R^{d},
(17) —S(O)ₘC₁₋₆alkyl, and
(18) —NR^{c}R^{d};
   R⁸ is selected from:

(1) hydrogen,

(2) halogen,

(3) C₁₋₁₀ alkyl,

(4) (CH₂)ₙ—O—C₁₋₆alkyl,

(5) (CH₂)ₙ—NH₂,

(6) (CH₂)ₙ—NH(C₁₋₆alkyl), and

(7) (CH₂)ₙ—N(C₁₋₆alkyl)₂,

wherein each alkyl is unsubstituted or substituted with one to four substituents independently selected from R^{a};
each R^{a} is independently selected from:

(1) —OR^{d},

(2) —NR^{c}S(O)ₘR^{d},

(3) halogen,

(4) —SR^{d},

(5) —S(O)ₘNR^{c}R^{d},

(6) —NR^{c}R^{d},

(7) —C(O)R^{d},

(8) —CO₂R^{d},

(9) —CN,

(10) —C(O)NR^{c}R^{d},

(11) —NR^{c}C(O)R^{d},

(12) —NR^{c}C(O)OR^{d},

(13) —NR^{c}C(O)NR^{c}R^{d},

(14) —CF₃, and

(15) —OCF₃;

each R^{b} is independently selected from:

(1) R^{a},

(2) oxo,

(3) C₁₋₁₀alkyl,

(4) C₂₋₁₀ alkenyl,

(5) cycloalkyl,

(6) cycloalkyl-C₁₋₁₀alkyl;

(7) cycloheteroalkyl,

(8) cycloheteroalkyl-C₁₋₁₀ alkyl,

(9) aryl,

(10) heteroaryl,

(11) aryl-C₁₋₁₀alkyl, and

(12) heteroaryl-C₁₋₁₀alkyl,

wherein alkyl and alkenyl are unsubstituted or substituted with one, two, three or four R^{h} substituents, and cycloalkyl, cycloheteroalkyl, aryl and heteroaryl are unsubstituted or substituted with one, two or three R^{h} substituents;
R^{c} and R^{d} are each independently selected from:

(1) hydrogen,

(2) C₁₋₁₀alkyl,

(3) C₂₋₁₀alkenyl,

(4) cycloalkyl,

(5) cycloalkyl-C₁₋₁₀alkyl-,

(6) cycloheteroalkyl,

(7) cycloheteroalkyl-C₁₋₁₀ alkyl-,

(8) aryl,

(9) heteroaryl,

(10) aryl-C₁₋₁₀alkyl-, and

(11) heteroaryl-C₁₋₁₀alkyl-,

wherein alkyl, alkenyl, cycloalkyl, cycloheteroalkyl, aryl and heteroaryl are unsubstituted or substituted with one to three substituents selected from R^{f};

each R^{e} is independently selected from:

(1) C₁₋₁₀alkyl,

(2) aryl,

(3) heteroaryl,

(4) cycloalkyl, and

(5) cycloheteroalkyl;

wherein alkyl, cycloalkyl, cycloheteroalkyl, aryl and heteroaryl are unsubstituted or substituted with one, two, or three substituents independently selected from R^{h};
each R^{f} is independently selected from:

(1) halogen,

(2) C₁₋₆alkyl,

(3) 4-methylbenzyl-,

(4) —OH,

(5) —O—C₁₋₄alkyl,

(6) benzyloxy-,

(7) -oxo,

(8) —OC(O)—C₁₋₆alkyl,

(9) —C(O)O—C₁₋₆alkyl,

(10) —S—C₁₋₄alkyl,

(11) —NH(CH₃),

(12) —N(CH₃)₂,

(13) —NO₂,

(14) —CN,

(15) —CF₃, and

(16) —OCF₃,

wherein alkyl may be unsubstituted or substituted with one, two or three substituents selected from R^{g};
each R^{g} is independently selected from:

(1) halogen,

(2) —O—C₁₋₄alkyl,

(3) —OH,

(4) —S—C₁₋₄alkyl,

(5) —CN,

(6) —CF₃, and

(7) —OCF₃;

each R^{h} is independently selected from:

(1) halogen,

(2) oxo,

(3) amino,

(4) hydroxy,

(5) C₁₋₄alkyl,

(6) —O—C₁₋₄alkyl,

(7) —S—C₁₋₄alkyl,

(8) —CN,

(9) —CF₃, and

(10) —OCF₃;

each m is independently selected from 0, 1 and 2; and
each n is independently selected from 0, 1, 2 and 3.

In one embodiment of the present invention, Z is selected from C═O, and CR⁸. In a class of this embodiment, Z is C═O. In another class of this embodiment, Z is CR⁸. In yet another class of this embodiment, Z is CH.

In one embodiment of the present invention, “a” is a single bond when R² is present and Z is C═O, or a double bond when R² is absent and Z is CR⁸.

In another embodiment of the present invention, “a” is a single bond, R² is present, and Z is C═O. In a class of this embodiment, “a” is a single bond, R² is hydrogen, and Z is C═O.

In another embodiment of the present invention, “a” is a double bond, R² is absent, and Z is CH.

In one embodiment of the present invention, Ar¹ is selected from: aryl, and heteroaryl, wherein aryl and heteroaryl are unsubstituted or substituted with one, two, three or four substituents selected from R⁴ and R⁵. In a class of this embodiment of the present invention, Ar¹ is selected from: aryl, and heteroaryl, wherein aryl and heteroaryl are substituted with R⁴ and R⁵.

In one class of this embodiment, Ar¹ is selected from phenyl, and pyridyl, wherein the phenyl and pyridyl are substituted with R⁴ and R⁵.

In a subclass of this class, Ar¹ is selected from:

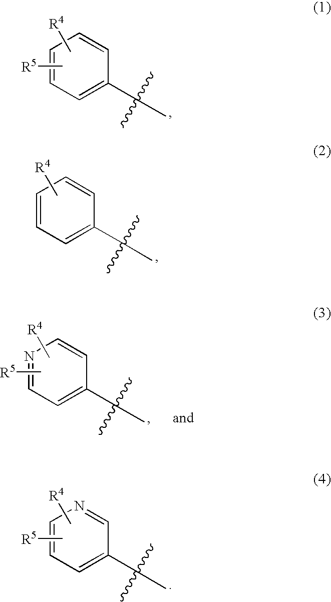

In a subclass of this class, Ar¹ is selected from:

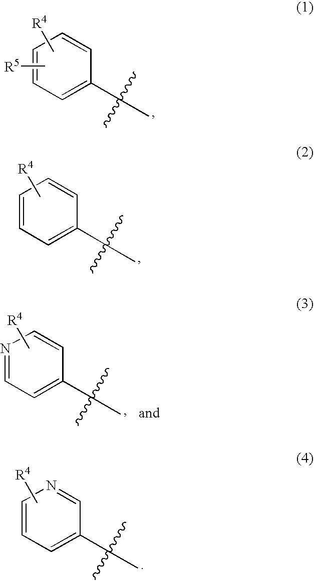

In another subclass of this class, Ar¹ is selected from:

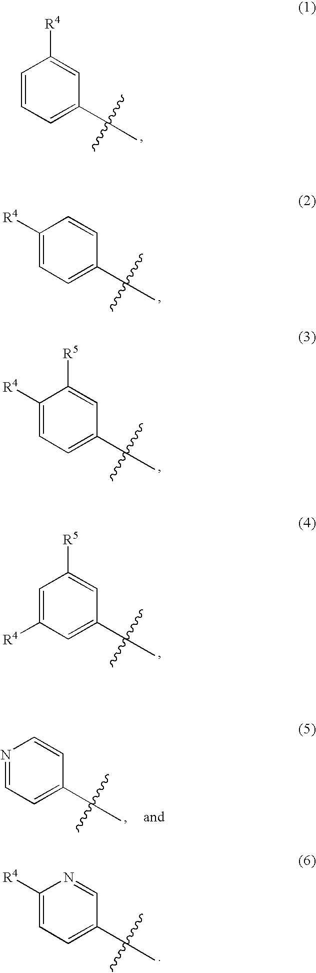

In another subclass, Ar¹ is selected from: 4-chlorophenyl, 4-bromophenyl, 4-fluorophenyl, 3,4-difluorophenyl, 3,5-difluorophenyl, 3-methyl-4-chlorophenyl, 4-methylsulfonylphenyl, 4-cyanophenyl, 4-methylphenyl, 4-trifluoromethylphenyl, 4-methoxyphenyl, 4-isopropyloxyphenyl, 4-trifluoromethyl-oxyphenyl, 4-(dimethylamine)phenyl, 3-chlorophenyl, and 4-(1,2,4-oxadiazole)phenyl. In yet another subclass, Ar¹ is 4-cyanophenyl.

In one embodiment of the present invention, Ar² is aryl, wherein aryl is unsubstituted or substituted with one, two, three or four substituents independently selected from R⁶ and R⁷.

In another embodiment of the present invention, Ar² is aryl, wherein aryl is substituted with R⁶ and R⁷. In one class of this embodiment, Ar² is phenyl, wherein phenyl is substituted with R⁶ and R⁷.

In another class of this embodiment, Ar² is selected from:

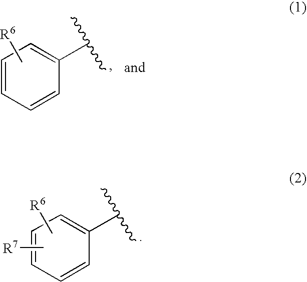

In another class of this embodiment, Ar² is selected from:

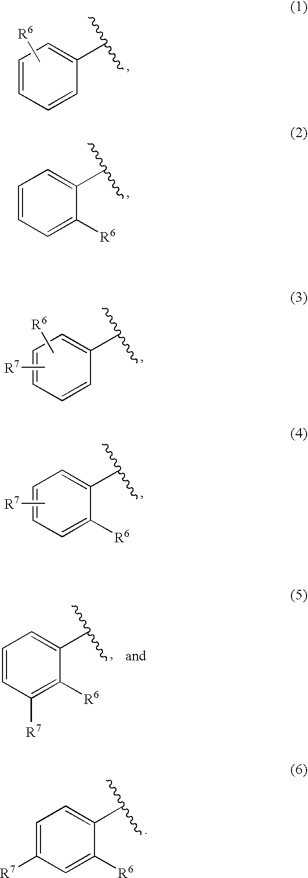

In another class of this embodiment, Ar² is selected from:

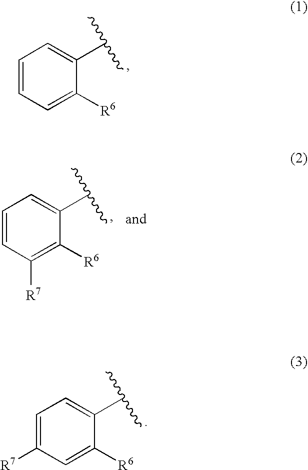

In yet another class, Ar² is selected from: 2-chlorophenyl, 2-bromophenyl, 2-methylphenyl, 4-bromo-2-chlorophenyl, 2-bromo-4-chlorophenyl, 4-cyano-2-chlorophenyl, 2,4-cyanophenyl, and 3-methyl-2-chlorophenyl. In a subclass of this class, Ar² is selected from 2-chlorophenyl.

In one embodiment of the present invention, R¹ is selected from: C₁₋₁₀alkyl, C₃₋₁₀cycloalkyl, C₃₋₁₀cycloalkenyl, C₃₋₁₀cycloalkyl-C₁₋₄alkyl-, C₃₋₁₀cycloalkenyl-C₁₋₄alkyl-, cycloheteroalkyl, cycloheteroalkyl-C₁₋₄alkyl-, aryl, aryl-C₁₋₄alkyl-, heteroaryl, heteroaryl-C₁₋₄alkyl, —OR^{e}, and —NR^{c}R^{d};

wherein each alkyl is unsubstituted or substituted with one to four substituents independently selected from R^{a}, and each cycloalkyl, cycloalkenyl, cycloheteroalkyl, aryl and heteroaryl is optionally substituted with one to four substituents independently selected from R^{b}.

In another embodiment of the present invention, R¹ is selected from: C₁₋₁₀alkyl, C₃₋₁₀cycloalkyl, C₃₋₁₀cycloalkenyl, C₃₋₁₀cycloalkyl-C₁₋₄alkyl-, C₃₋₁₀cycloalkenyl-C₁₋₄alkyl-, cycloheteroalkyl, cycloheteroalkyl-C₁₋₄alkyl-, aryl, aryl-C₁₋₄alkyl-, heteroaryl, heteroaryl-C₁₋₄alkyl, —OR^{e}, and —NR^{c}R^{d}.

In another embodiment of the present invention, R¹ is C₁₋₁₀alkyl or C₃₋₁₀cycloalkyl, wherein alkyl and cycloalkyl are unsubstituted or substituted with one to four substituents independently selected from R^{a}.

In another embodiment of the present invention, R¹ is C₁₋₁₀alkyl, wherein alkyl is unsubstituted or substituted with one to four substituents independently selected from R^{a}. In a class of this embodiment, R¹ is tert-butyl or isopropyl, wherein tert-butyl and isopropyl are unsubstituted or substituted with one to four substituents independently selected from R^{a}. In another class of this embodiment, R¹ is tert-butyl or isopropyl.

In one embodiment of the present invention, R² is absent or present and selected from: hydrogen, and C₁₋₁₀ alkyl. In a class of this embodiment, R² is absent. In another class of this embodiment, R² is hydrogen, or C₁₋₁₀alkyl. In another class of this embodiment, R² is hydrogen. In another class of this embodiment, R² is C₁₋₁₀alkyl. In another class of this embodiment, R² is absent or R² is hydrogen. In a class of this embodiment, R² is absent when “a” is a double bond and Z is CH. In another class of this embodiment, R² is hydrogen when “a” is a single bond and Z is C═O.

In one embodiment of the present invention, R³ is hydrogen.

In one embodiment of the present invention, each R⁴, R⁵, R⁶, and R⁷ is independently selected from: hydrogen, halogen, —CN, —C₁₋₆alkyl, unsubstituted or substituted with one, two or three R^{f} substitutents, —CF₃, —C₂₋₆alkenyl, unsubstituted or substituted with one, two or three R^{f} substitutents, -cycloalkyl, unsubstituted or substituted with one, two or three R^{f} substitutents, cycloalkyl-C₁₋₃alkyl-, unsubstituted or substituted with one, two or three R^{f} substitutents, -cycloheteroalkyl, unsubstituted or substituted with one, two or three R^{f} substitutents, -aryl, unsubstituted or substituted with one, two or three R^{h} substitutents, aryl-C₁₋₃alkyl-, unsubstituted or substituted on aryl with one, two or three R^{h} substitutents, -heteroaryl, unsubstituted or substituted with one, two or three R^{h} substitutents, heteroaryl-C₁₋₃alkyl-, unsubstituted or substituted with one, two or three R^{h} substitutents, —OR^{d}, —OCF₃, —C(O)R^{d}, —S(O)ₘC₁₋₆alkyl, and —NR^{c}R^{d}.

In another embodiment of the present invention, each R⁴, R⁵, R⁶, and R⁷ is independently selected from: -hydrogen, -halogen, and —C₁₋₆alkyl, unsubstituted or substituted with one, two or three R^{f} substitutents. In one class of this embodiment, each R⁴, R⁵, R⁶, and R⁷ is independently selected from:

-hydrogen, -halogen, and —C₁₋₆alkyl. In another class of this embodiment, each R⁴, R⁵, R⁶, and R⁷ is independently selected from: -hydrogen, and -halogen.

In another embodiment of the present invention, each R⁴, R⁵, R⁶, and R⁷ is independently selected from: hydrogen, -halogen, —CN, —C₁₋₆alkyl, unsubstituted or substituted with one, two or three R^{f} substitutents, —CF₃, -heteroaryl, unsubstituted or substituted with one, two or three R^{h} substitutents, —OR^{d}, —OCF₃, —C(O)R^{d}, —S(O)ₘC₁₋₆alkyl, and —NR^{c}R^{d}.

In another embodiment of the present invention, each R⁴ and R⁵ is independently selected from:

-hydrogen, -halogen, —CN, —C₁₋₆alkyl, unsubstituted or substituted with one, two or three R^{f} substitutents, —CF₃, -heteroaryl, unsubstituted or substituted with one, two or three R^{h} substitutents, —OC₁₋₆alkyl, —OCF₃, —C(O)C₁₋₆alkyl, —S(O)₂CH₃, and —N(C₁₋₆alkyl)₂. In a class of this embodiment, each R⁴ and R⁵ is independently selected from: -hydrogen, —Br, —Cl, —F, —CN, —CH₃, —CF₃, -oxadiazole, —OCH₃, —OCH(CH₃)₂, —OCF₃, —C(O)CH₃, —S(O)₂CH₃, and —N(CH₃)₂.

In another embodiment of the present invention, each R⁴ is independently selected from: -hydrogen, -halogen, —CN, —C₁₋₆alkyl-, unsubstituted or substituted with one, two or three R^{f} substitutents, —CF₃, -heteroaryl, unsubstituted or substituted with one, two or three R^{h} substitutents, —OC₁₋₆alkyl, —OCF₃, —S(O)₂CH₃, and —N(C₁₋₆alkyl)₂. In a class of this embodiment, each R⁴ is independently selected from: -hydrogen, —Br, —Cl, —F, —CN, —CH₃, —CF₃, -oxadiazole, —OCH₃, —OCH(CH₃)₂, —OCF₃, —S(O)₂CH₃, and —N(CH₃)₂.

In another embodiment of the present invention, R⁵ is independently selected from: -hydrogen, -halogen, and —C₁₋₆alkyl, unsubstituted or substituted with one, two or three R^{f} substitutents. In a class of this embodiment, R⁵ is independently selected from: -hydrogen, —F, and —CH₃.

In another embodiment of the present invention, each R⁶ and R⁷ is independently selected from: -hydrogen, -halogen, —CN, —C₁₋₆alkyl, unsubstituted or substituted with one, two or three R^{f} substitutents, -aryl, unsubstituted or substituted with one, two or three R^{h} substitutents, and —C(O)R^{d}. In a class of this embodiment, each R⁶ and R⁷ is independently selected from: -hydrogen, —Cl, —Br, —CN, —CH₃, 4-fluorophenyl, and —C(O)CH₃.

In another embodiment of the present invention, each R⁶ is independently selected from: -hydrogen, -halogen, —CN, —C₁₋₆alkyl, unsubstituted or substituted with one, two or three R^{f} substitutents, and aryl, unsubstituted or substituted with one, two or three R^{h} substitutents. In a class of this embodiment, each R⁶ is independently selected from: -hydrogen, —Cl, —Br, —CN, —CH₃, and 4-fluorophenyl.

In another embodiment of the present invention, each R⁷ is independently selected from: -hydrogen, -halogen, —CN, —C₁₋₆alkyl, unsubstituted or substituted with one, two or three R^{f} substitutents, and —C(O)R^{d}. In a class of this embodiment, each R⁷ is independently selected from: -hydrogen, —Cl, —Br, —CN, —CH₃, and —C(O)CH₃.

In one embodiment of the present invention, R⁸ is selected from: hydrogen, halogen, C₁₋₁₀alkyl, (CH₂)ₙ—O—C₁₋₆alkyl, (CH₂)ₙ—NH₂, (CH₂)ₙ—NH(C₁₋₆alkyl), and (CH₂)ₙ—N(C₁₋₆alkyl)₂, wherein each alkyl is unsubstituted or substituted with one to four substituents independently selected from R^{a}.

In another embodiment of the present invention, R⁸ is selected from: hydrogen, halogen, C₁₋₁₀alkyl, (CH₂)ₙ—O—C₁₋₆alkyl, (CH₂)ₙ—NH₂, (CH₂)ₙ—NH(C₁₋₆alkyl), and (CH₂)ₙ—N(C₁₋₆alkyl)₂. In a class of this embodiment, R⁸ is selected from: hydrogen, halogen, C₁₋₆alkyl, —O—C₁₋₆alkyl, —NH₂, —NH(C₁₋₆alkyl), and —N(C₁₋₆alkyl)₂. In another class of this embodiment, R⁸ is hydrogen.

In one embodiment, when Ar¹ is phenyl, pyridine, pyrimidine or bromide and Ar² is phenyl, at least one of R⁴, R⁵, R⁶ and R⁷ is not hydrogen. In a class of this embodiment, when Ar¹ is phenyl, pyridine, pyrimidine or bromide and Ar² is phenyl, at least one of R⁶ and R⁷ is not hydrogen.

In one embodiment of the present invention, each R^{a} is independently selected from: —OR^{d}, —NR^{c}S(O)ₘR^{d}, halogen, —SR^{d}, —S(O)ₘNR^{c}R^{d}, —NR^{c}R^{d}, —C(O)R^{d}, —CO₂R^{d}, —CN, —C(O)NR^{c}R^{d}, —NR^{c}C(O)R^{d}, —NR^{c}C(O)OR^{d}, —NR^{c}C(O)NR^{c}R^{d}, —CF₃, and —OCF₃.

In another embodiment of the present invention, each R^{a} is independently selected from: —OR^{d}, —NR^{c}S(O)ₘR^{d}, halogen, —SR^{d}, —S(O)ₘNR^{d}, —S(O)ₘNR^{c}R^{d}, —NR^{c}R^{d}, —C(O)R^{d}, —CO₂R^{d}, —CN, —C(O)NR^{c}R^{d}, —NR^{c}C(O)R^{d}, —NR^{c}C(O)OR^{d}, —NR^{c}C(O)NR^{c}R^{d}, —CF₃, and —OCF₃. In one class of this embodiment, each R^{a} is independently selected from: —OH, —OCH₃, halogen, —SH, —SO₂R^{d}, —NH₂, —CN, —CO₂R^{d}, —C(O)NR^{c}R^{d}, —CF₃, and —OCF₃. In one subclass, each R^{a} is independently selected from: —OH, —F, —SO₂CH₃, —CO₂—C₁₋₆alkyl, and —CF₃. In another subclass, each R^{a} is independently selected from: —OH, —F, and —CF₃. In another class, R^{a} is halogen.

In one embodiment of the present invention, each R^{b} is independently selected from: R^{a}, oxo, C₁₋₁₀alkyl, C₂₋₁₀ alkenyl, cycloalkyl, cycloalkyl-C₁₋₁₀alkyl, cycloheteroalkyl, cycloheteroalkyl-C₁₋₁₀ alkyl, aryl, heteroaryl, aryl-C₁₋₁₀alkyl, and heteroaryl-C₁₋₁₀alkyl, wherein alkyl and alkenyl moieties are unsubstituted or substituted with one, two, three or four R^{h} substituents, and cycloalkyl, cycloheteroalkyl, aryl and heteroaryl moieties are unsubstituted or substituted with one, two or three R^{h} substituents.

In another embodiment of the present invention, each R^{b} is independently selected from: R^{a}, oxo, C₁₋₁₀alkyl, C₂₋₁₀ alkenyl, cycloalkyl, cycloalkyl-C₁₋₁₀alkyl-, cycloheteroalkyl, cycloheteroalkyl-C₁₋₁₀ alkyl-, aryl, heteroaryl, aryl-C₁₋₁₀ alkyl, and heteroaryl-C₁₋₁₀alkyl-.

In another embodiment, R^{b} is independently selected from: —OR^{d}, —NR^{c}S(O)ₘR^{d}, halogen, —SR^{d}, —S(O)ₘNR^{c}R^{d}, —NR^{c}R^{d}, —C(O)R^{d}, —CO₂R^{d}, —CN, —C(O)NR^{c}R^{d}, —NR^{c}C(O)R^{d}, —NR^{c}C(O)OR^{d}, —NR^{c}C(O)NR^{c}R^{d}, —CF₃, —OCF₃, oxo, C₁₋₁₀alkyl, C₂₋₁₀ alkenyl, cycloalkyl, cycloalkyl-C₁₋₁₀ alkyl, cycloheteroalkyl, cycloheteroalkyl-C₁₋₁₀ alkyl, aryl, heteroaryl, aryl-C₁₋₁₀alkyl, and heteroaryl-C₁₋₁₀alkyl, wherein alkyl and alkenyl moieties are unsubstituted or substituted with one, two, three or four R^{h} substituents, and cycloalkyl, cycloheteroalkyl, aryl and heteroaryl moieties are unsubstituted or substituted with one, two or three R^{h} substituents. In one class of this embodiment, each R^{b} is independently selected from: —OR^{d}, halogen, —SCH₃, —NR^{c}R^{d}, —C(O)R^{d}, —CO₂R^{d}, —CN, —C(O)NR^{c}R^{d}, —CF₃, —OCF₃, oxo, C₁₋₁₀alkyl, C₂₋₁₀ alkenyl, cycloalkyl, cycloalkyl-methyl, cycloheteroalkyl, cycloheteroalkyl-methyl, aryl, heteroaryl, aryl-methyl, heteroaryl-methyl, wherein alkyl and alkenyl moieties are unsubstituted or substituted with one, two, or three R^{h} substituents, and cycloalkyl, cycloheteroalkyl, aryl and heteroaryl moieties are unsubstituted or substituted with one, two or three R^{h} substituents. In another class of this embodiment, each R^{b} is independently selected from: —OH, —OCH₃, halogen, —N(CH₃)₂, —CH(O)—C(O)R^{d}, —CO₂CH₃, —CO₂CH₂C₆H₅, —CN, —CF₃, —OCF₃, oxo, C₁₋₃alkyl, C₂₋₃ alkenyl, cyclopropyl, oxadiazolyl, pyrazolyl, tetrazolyl, and phenyl, wherein alkyl and alkenyl moieties are unsubstituted or substituted with one, two, or three R^{h} substituents, and cycloalkyl, cycloheteroalkyl, aryl and heteroaryl moieties are unsubstituted or substituted with one, two or three R^{h} substituents. In another class of this embodiment, R^{b} is independently selected from: —OH, —OCH₃, halogen, —C(O)CH₃, —CO₂CH₃, —CN, —CF₃, —OCF₃, oxo, methyl, ethyl, isopropyl, and C₂₋₃ alkenyl, wherein alkyl and alkenyl moieties are unsubstituted or substituted with one, two, or three R^{h} substituents. In a subclass, each R^{b} is independently selected from: —OH, fluoro, —C(O)CH₃, —CO₂CH₃, —CN, —CF₃, —OCF₃, oxo, methyl, ethyl, isopropyl, and C₂₋₃ alkenyl, wherein alkyl and alkenyl moieties are unsubstituted or substituted with one, two, or three R^{h} substituents. In another subclass, each R^{b} is independently selected from: —OH, fluoro, oxo, methyl, ethyl, isopropyl, and C₂₋₃ alkenyl, wherein alkyl and alkenyl moieties are unsubstituted or substituted with one, two, or three R^{h} substituents.

In one embodiment of the present invention, R^{c} and R^{d} are each independently selected from: hydrogen, C₁₋₁₀alkyl, C₂₋₁₀ alkenyl, cycloalkyl, cycloalkyl-C₁₋₁₀alkyl-, cycloheteroalkyl, cycloheteroalkyl-C₁₋₁₀ alkyl-, aryl, heteroaryl, aryl-C₁₋₁₀ alkyl-, and heteroaryl-C₁₋₁₀alkyl-.

In another embodiment of the present invention, R^{c} and R^{d} are each independently selected from: hydrogen, C₁₋₁₀alkyl, C₂₋₁₀ alkenyl, cycloalkyl, cycloalkyl-C₁₋₁₀alkyl-, cycloheteroalkyl, cycloheteroalkyl-C₁₋₁₀ alkyl-, aryl, heteroaryl, aryl-C₁₋₁₀alkyl-, and heteroaryl-C₁₋₁₀ alkyl-, wherein each R^{c} and R^{d} moiety, other than hydrogen, may be unsubstituted or substituted with one to three substituents selected from R^{f}.

In another embodiment, R^{c} and R^{d} are each independently selected from: hydrogen, and C₁₋₁₀ alkyl, wherein C₁₋₁₀ alkyl may be unsubstituted or substituted with one to three substituents selected from R^{f}, In a class of this embodiment, R^{c} and R^{d} are each C₁₋₁₀ alkyl, wherein C₁₋₁₀ alkyl may be unsubstituted or substituted with one to three substituents selected from R^{f}. In a subclass of this class, R^{c} and R^{d} are each C₁₋₁₀alkyl. In another class of this embodiment, each R^{c}C is independently selected from: hydrogen, and C₁₋₃alkyl, unsubstituted or substituted with one to three substituents selected from R^{f}. In a subclass of this class, each R^{c} is independently selected from: hydrogen, and C₁₋₃alkyl. In another subclass, each R^{c} is independently selected from: hydrogen, and methyl. In another subclass, R^{c} is methyl. In yet another subclass, R^{c} is hydrogen. In another class of this embodiment, each R^{d} is independently selected from: hydrogen, and C₁₋₃alkyl, unsubstituted or substituted with one to three substituents selected from R^{f}. In a subclass of this class, each R^{d} is independently selected from: hydrogen, and C₁₋₃alkyl. In another subclass, each R^{d} is independently selected from: hydrogen, and methyl. In another subclass, R^{d} is methyl. In yet another subclass, R^{d} is hydrogen. In another class, each R^{d} is independently selected from: hydrogen, C₁₋₆alkyl, C₂₋₆alkenyl, cycloalkyl, cycloalkyl-C₁₋₁₀alkyl-, cycloheteroalkyl, cycloheteroalkyl-C₁₋₁₀ alkyl-, aryl, heteroaryl, aryl-C₁₋₃alkyl-, and heteroaryl-C₁₋₃alkyl-, wherein each R^{d} moiety, other than hydrogen, may be unsubstituted or substituted with one, two or three substituents selected from R^{f}.

In one embodiment of the present invention, each R^{e} is independently selected from: C₁₋₁₀alkyl, aryl, heteroaryl, cycloalkyl, and cycloheteroalkyl.

In another embodiment of the present invention, each R^{e} is independently selected from: C₁₋₁₀alkyl, aryl, heteroaryl, cycloalkyl, and cycloheteroalkyl; wherein alkyl and aryl are unsubstituted or substituted with one, two, or three substituents independently selected from R^{f}.

In another embodiment of the present invention, each R^{e} is independently selected from: C₁₋₁₀alkyl, wherein alkyl is unsubstituted or substituted with one, two, or three substituents independently selected from R^{h}. In a class of this embodiment, each R^{e} is independently selected from: C₁₋₁₀alkyl.

In one embodiment of the present invention, each R^{f} is independently selected from: halogen, C₁₋₆alkyl, 4-methylbenzyl-, —OH, —O—C₁₋₄alkyl, benzyloxy-, -oxo, —OC(O)—C₁₋₆alkyl, —C(O)O—C₁₋₆alkyl, —S—C₁₋₄alkyl, —NH(CH₃), —N(CH₃)₂, —NO₂, —CN, —CF₃, and —OCF₃.

In another embodiment of the present invention, each R^{f} is independently selected from: halogen, C₁₋₆alkyl, 4-methylbenzyl-, —OH, —O—C₁₋₄alkyl, benzyloxy-, -oxo, —OC(O)—C₁₋₆alkyl, —C(O)O—C₁₋₆alkyl, —S—C₁₋₄alkyl, —NH₂, —NH(CH₃), —N(CH₃)₂, —NO₂, —CN, —CF₃, and —OCF₃; wherein alkyl may be unsubstituted or substituted with one, two or three substituents selected from R^{g}.

In one embodiment of the present invention, each R^{g} is independently selected from: halogen, —O—C₁₋₄alkyl, —OH, —S—C₁₋₄alkyl, —CN, —CF₃, and —OCF₃.

In one class, each R^{g} is independently selected from: halogen, methoxy, —OH, —S—CH₃, —CN, —CF₃, and —OCF₃.

In one embodiment of the present invention, each R^{h} is independently selected from: halogen, oxo, amino, hydroxy, C₁₋₄alkyl, —O—C₁₋₄alkyl, —S—C₁₋₄alkyl, —CN, —CF₃, and —OCF₃.

In one class, each R^{h} is independently selected from: halogen, oxo, amino, hydroxy, C₁₋₄alkyl, —O—CH₃, —S—CH₃, —CN, —CF₃, and —OCF₃. In another class of this embodiment, each R^{h} is halogen.

In one embodiment of the present invention, each m is selected from 0, 1 and 2. In one class, m is 0. In another class, m is 1. In another class, m is 2.

In one embodiment of the present invention, each n is selected from 0, 1, 2 and 3. In one class, n is 0. In another class, n is 1. In another class, n is 2. In another class, n is 3.

One embodiment of the present invention comprises a compound of structural formula IA:

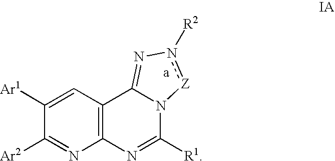

Another embodiment of the present invention comprises a compound of structural formula IB:

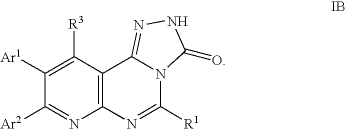

Another embodiment of the present invention comprises a compound of structural formula IC:

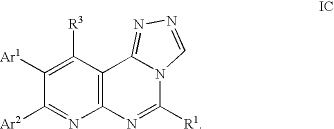

Another embodiment of the present invention comprises a compound of structural formula ID:

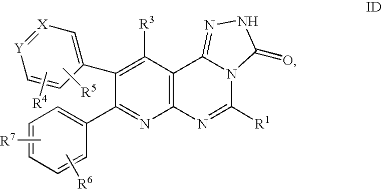

wherein X is CH or N; Y is CH or N, provided that at least one of X and Y is CH, and R¹, R³, R⁴, R⁵, R⁶ and R⁷ are a defined above.

Another embodiment of the present invention comprises a compound of structural formula IE:

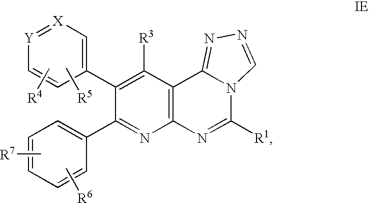

wherein X is CH or N; Y is CH or N provided that at least one of X and Y is CH, and R¹, R³, R⁴, R⁵, R⁶ and R⁷ are a defined above.

“Alkyl”, as well as other groups having the prefix “alk”, such as alkoxy, alkanoyl, means carbon chains of up to 10 carbons which may be linear or branched or combinations thereof. Examples of alkyl groups include methyl, ethyl, n-propyl, isopropyl, butyl, isobutyl, sec- and tert-butyl, pentyl, hexyl, heptyl, octyl, nonyl, and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means mono- or bicyclic or bridged saturated carbocyclic rings, each having from 3 to 10 carbon atoms. Examples of cycloalkyl include cyclopropyl, cyclobutyl, cyclopentyl, cyclohexyl, cycloheptyl, cyclooxtyl, tetrahydronaphthyl, decahydronaphthyl, bicyclo and the like. In one embodiment of the present invention, cycloalkyl is selected from cyclopropyl, cyclobutyl, cyclopentyl, cyclohexyl, and 1,2,3,4-tetrahydronaphthyl.

“Cycloalkenyl” means nonaromatic, mono- or bicyclic or bridged carbocyclic rings, each having from 3 to 10 carbon atoms and at least one degree of unsaturation. Examples of cycloalkyl include cyclopropenyl, cyclobutenyl, cyclopentenyl, cyclohexenyl, cycloheptenyl, cyclooxtenyl, decahydronaphthyl, bicyclo[2.2.1]hept-5-en-2-yl, and the like. In one embodiment of the present invention, cycloalkenyl is selected from cyclopropenyl, cyclobutenyl, cyclopentenyl, cyclohexenyl, and bicyclo[2.2.1]hept-5-en-2-yl, and the like.

“Aryl” means mono- or bicyclic aromatic rings containing only carbon atoms. Examples of aryl include phenyl, naphthyl, and the like.

“Heteroaryl” means an aromatic or partially aromatic heterocycle that contains at least one ring heteratom selected from O, S, and N. Heteroaryls thus include heteroaryls fused to other kinds of rings, such as aryls, cycloalkyls, and cycloheteroalkyls that are not aromatic. Examples of heteroaryl groups include: pyrrolyl, isoxazolyl, isothiazaolyl, pyrazolyl, pyridyl, oxazolyl, oxadiazolyl, thiadiazolyl, thiazolyl, imidazolyl, triazolyl, tetrazolyl, furyl, triazinyl, thienyl, pyrimidyl, pyridazinyl, pyrazinyl, benzoxazolyl, benzothiazolyl, benzimidazolyl, benzofuranyl, dibenzylfuranyl, isobenzylfuranyl, benzopyrazolyl, benzothienyl, benzothiazolyl, furo(2,3-b)pyridyl, quinolyl, indolyl, isoquinolyl, oxazolidinyl, imidazothiathiazolyl, pyrazolylpyridyl, benzotriazolyl, methylenedioxyphenyl, hexahydrothieno-pyridinyl, thienopyridinyl, and the like. In one embodiment of the present invention, heteroaryl is selected from pyridyl, furyl, thienyl, pyrazolyl, oxazolyl, isoxazolyl, oxadiazolyl, indazolyl, oxadiazolyl, tetrazolyl, imidazolyl, indolyl, benzimidazolyl, triazolyl, and benzopyrazolyl.

“Cycloheteroalkyl” refers to a saturated or unsaturated non-aromatic ring or ring system containing at least one heteroatom selected from O, S and N, further including the oxidized forms of sulfur, namely SO and SO₂, in which the point of attachment may be carbon or nitrogen. Examples of heterocycloalkyl include tetrahydrofuranyl, azetidinyl, perhydroazepinyl, dihydrofuranyl, dioxanyl, oxanyl, morpholinyl, 1,4-dithianyl, piperazinyl, piperidinyl, 1,3-dioxolanyl, imidazolidinyl, imidazolinyl, pyrrolinyl, pyrrolidinyl, pyranyl, tetrahydropyranyl, dihydropyranyl, oxathiolanyl, dithiolanyl, 1,3-dithianyl, oxathianyl, thiomorpholinyl, dioxidoisothiazolidinyl, azacycloheptyl, diazobicyclo[3.2.1]-octane, and hexahydroindazolyl. The cycloheteroalkyl ring may be substituted on the ring carbons and/or the ring nitrogens. In one embodiment of the present invention, cycloheteroalkyl is selected from tetrahydrofuranyl, imidazolidinyl, piperidinyl, pyrrolidinyl, isothiazolidinyl morpholinyl and thiomorpholinyl.

“Halogen” includes fluorine, chlorine, bromine and iodine.

When any variable (e.g., R¹, R^{d}, etc.) occurs more than one time in any constituent or in formula I, its definition on each occurrence is independent of its definition at every other occurrence. Also, combinations of substituents and/or variables are permissible only if such combinations result in stable compounds. A squiggly line across a bond in a substituent variable represents the point of attachment.

Under standard nomenclature used throughout this disclosure, the terminal portion of the designated side chain is described first, followed by the adjacent functionality toward the point of attachment. For example, a C₁₋₅ alkylcarbonylamino C₁₋₆ alkyl substituent is equivalent to:

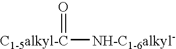

In choosing compounds of the present invention, one of ordinary skill in the art will recognize that the various substituents, i.e. R¹, R², etc., are to be chosen in conformity with well-known principles of chemical structure connectivity and stability.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Compounds of Formula I may contain one or more asymmetric centers and can thus occur as racemates and racemic mixtures, single enantiomers, diastereomeric mixtures and individual diastereomers. The present invention is meant to comprehend all such isomeric forms of the compounds of Formula I.

Some of the compounds described herein contain olefinic double bonds, and unless specified otherwise, are meant to include both E and Z geometric isomers.

Tautomers are defined as compounds that undergo rapid proton shifts from one atom of the compound to another atom of the compound. Some of the compounds described herein may exist as tautomers with different points of attachment of hydrogen. Such an example may be a ketone and its enol form known as keto-enol tautomers. The individual tautomers as well as mixture thereof are encompassed with compounds of Formula I.

Compounds of the Formula I may be separated into diastereoisomeric pairs of enantiomers by, for example, fractional crystallization from a suitable solvent, for example MeOH or ethyl acetate or a mixture thereof. The pair of enantiomers thus obtained may be separated into individual stereoisomers by conventional means, for example by the use of an optically active amine as a resolving agent or on a chiral HPLC column.

Alternatively, any enantiomer of a compound of the general Formula I may be obtained by stereospecific synthesis using optically pure starting materials or reagents of known configuration.

Furthermore, some of the crystalline forms for compounds of the present invention may exist as polymorphs and as such are intended to be included in the present invention. In addition, some of the compounds of the instant invention may form solvates with water or common organic solvents. Such solvates are encompassed within the scope of this invention.

It is generally preferable to administer compounds of the present invention as enantiomerically pure formulations. Racemic mixtures can be separated into their individual enantiomers by any of a number of conventional methods. These include chiral chromatography, derivatization with a chiral auxiliary followed by separation by chromatography or crystallization, and fractional crystallization of diastereomeric salts.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations.

It will be understood that, as used herein, references to the compounds of Formula I are meant to also include the pharmaceutically acceptable salts.

Compounds of the present invention are modulators of the CB1 receptor. In particular, the compounds of structural formula I are antagonists or inverse agonists of the CB1 receptor.

An “agonist” is a compound (hormone, neurotransmitter or synthetic compound) which binds to a receptor and mimics the effects of the endogenous regulatory compound, such as contraction, relaxation, secretion, change in enzyme activity, etc. An “antagonist” is a compound, devoid of intrinsic regulatory activity, which produces effects by interfering with the binding of the endogenous agonist or inhibiting the action of an agonist. An “inverse agonist” is a compound which acts on a receptor but produces the opposite effect produced by the agonist of the particular receptor.

Compounds of this invention are modulators of the CB1 receptor and as such are useful as centrally acting drugs in the treatment of psychosis, memory deficits, cognitive disorders, Alzheimer's disease, migraine, neuropathy, neuro-inflammatory disorders including multiple sclerosis and Guillain-Barre syndrome and the inflammatory sequelae of viral encephalitis, cerebral vascular accidents, and head trauma, anxiety disorders, stress, epilepsy, Parkinson's disease, movement disorders, and schizophrenia. In particular, the compounds of this invention are antagonists/inverse agonists of the CB1 receptor. The compounds are also useful for the treatment of substance abuse disorders, particularly to opiates, alcohol, marijuana, and nicotine. In particular, the compounds of the invention are useful for smoking cessation. The compounds are also useful for the treatment of obesity or eating disorders associated with excessive food intake and complications associated therewith, including left ventricular hypertrophy, as well as treating or preventing obesity in other mammalian species, including canines and felines. The compounds are also useful for the treatment of constipation and chronic intestinal pseudo-obstruction. The compounds are also useful for the treatment of cirrhosis of the liver, non-alcoholic fatty liver disease (NAFLD), non-alcoholic steatohepatitis (NASH), promotion of wakefulness and treatment of asthma.

The terms “administration of” and or “administering a” compound should be understood to mean providing a compound of the invention or a prodrug of a compound of the invention to the individual in need of treatment.

The administration of the compound of structural formula I in order to practice the present methods of therapy is carried out by administering an effective amount of the compound of structural formula I to the mammalian patient in need of such treatment or prophylaxis. The need for a prophylactic administration according to the methods of the present invention is determined via the use of well known risk factors. The effective amount of an individual compound is determined, in the final analysis, by the physician or veterinarian in charge of the case, but depends on factors such as the exact disease to be treated, the severity of the disease and other diseases or conditions from which the patient suffers, the chosen route of administration other drugs and treatments which the patient may concomitantly require, and other factors in the physician's judgment.

The usefulness of the present compounds in these diseases or disorders may be demonstrated in animal disease models that have been reported in the literature. The following are examples of such animal disease models: a) suppression of food intake and resultant weight loss in rats (Life Sciences 1998, 63, 113-117); b) reduction of sweet food intake in marmosets (Behavioural Pharm. 1998, 9, 179-181); c) reduction of sucrose and ethanol intake in mice (Psychopharm. 1997, 132, 104-106); d) increased motor activity and place conditioning in rats (Psychopharm. 1998, 135, 324-332; Psychopharmacol 2000, 151: 25-30); e) spontaneous locomotor activity in mice (J. Pharm. Exp. Ther. 1996, 277, 586-594); f) reduction in opiate self-administration in mice (Sci. 1999, 283, 401-404); g) bronchial hyperresponsiveness in sheep and guinea pigs as models for the various phases of asthma (for example, see W. M. Abraham et al., “α₄-Integrins mediate antigen-induced late bronchial responses and prolonged airway hyperresponsiveness in sheep.” J. Clin. Invest. 93, 776 (1993) and A. A. Y. Milne and P. P. Piper, “Role of VLA-4 integrin in leucocyte recruitment and bronchial hyperresponsiveness in the guinea-pig.” Eur. J. Pharmacol., 282, 243 (1995)); h) mediation of the vasodilated state in advanced liver cirrhosis induced by carbon tetrachloride (Nature Medicine, 2001, 7 (7), 827-832); i) amitriptyline-induced constipation in cynomolgus monkeys is beneficial for the evaluation of laxatives (Biol. Pharm. Bulletin (Japan), 2000, 23(5), 657-9); j) neuropathology of paediatric chronic intestinal pseudo-obstruction and animal models related to the neuropathology of paediatric chronic intestinal pseudo-obstruction (Journal of Pathology (England), 2001, 194 (3), 277-88).

The magnitude of prophylactic or therapeutic dose of a compound of Formula I will, of course, vary with the nature of the severity of the condition to be treated and with the particular compound of Formula I and its route of administration. It will also vary according to the age, weight and response of the individual patient. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for intravenous administration is employed, a suitable dosage range is from about 0.001 mg to about 100 mg in one embodiment from about 0.01 mg to about 50 mg, and in another embodiment from 0.1 mg to 10 mg of a compound of Formula I per kg of body weight per day.

In the case where an oral composition is employed, a suitable dosage range is, e.g. from about 0.01 mg to about 1000 mg of a compound of Formula I per day. In one embodiment, the range is from about 0.1 mg to about 10 mg per day. For oral administration, the compositions are preferably provided in the form of tablets containing from 0.01 to 1,000 mg, preferably 0.01, 0.05, 0.1, 0.5, 1, 2, 2.5, 3, 4, 5, 6, 7, 8, 9, 10, 12, 12.5, 15, 20, 25, 30, 40, 50, 100, 250, 500, 750 or 1000 milligrams of the active ingredient for the symptomatic adjustment of the dosage to the patient to be treated.

Another aspect of the present invention provides pharmaceutical compositions which comprises a compound of Formula I and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

Any suitable route of administration may be employed for providing a mammal, particularly a human or companion animal such as a dog or cat, with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of Formula I as an active ingredient or a pharmaceutically acceptable salt thereof, and may also contain a pharmaceutically acceptable carrier and optionally other therapeutic ingredients. By “pharmaceutically acceptable” it is meant the carrier, diluent or excipient must be compatible with the other ingredients of the formulation and not deleterious to the recipient thereof. The compositions include compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form of an aerosol spray presentation from pressurized packs or nebulizers, or as powders which may be formulated and the powder composition may be inhaled with the aid of an insufflation powder inhaler device. The preferred delivery systems for inhalation are metered dose inhalation (MDI) aerosol, which may be formulated as a suspension or solution of a compound of Formula I in suitable propellants, such as fluorocarbons or hydrocarbons and dry powder inhalation (DPI) aerosol, which may be formulated as a dry powder of a compound of Formula I with or without additional excipients.

Suitable topical formulations of a compound of formula I include transdermal devices, aerosols, creams, solutions, ointments, gels, lotions, dusting powders, and the like. The topical pharmaceutical compositions containing the compounds of the present invention ordinarily include about 0.005% to 5% by weight of the active compound in admixture with a pharmaceutically acceptable vehicle. Transdermal skin patches useful for administering the compounds of the present invention include those well known to those of ordinary skill in that art.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the compounds of Formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules (including timed release and sustained release formulations), pills, cachets, powders, granules or tablets each containing a predetermined amount of the active ingredient, as a powder or granules or as a solution or a suspension in an aqueous liquid, a non-aqueous liquid, an oil-in-water emulsion or a water-in-oil liquid emulsion, including elixirs, tinctures, solutions, suspensions, syrups and emulsions. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet cachet or capsule contains from about 0.01 to 1,000 mg, particularly 0.01, 0.05, 0.1, 0.5, 1.0, 2, 2.5, 3, 4, 5, 6, 7, 8, 9, 10, 12, 15, 25, 30, 40, 50, 75, 100, 125, 150, 175, 180, 200, 225, 250, 500, 750 and 1,000 milligrams of the active ingredient for the symptomatic adjustment of the dosage to the patient to be treated.

Additional suitable means of administration of the compounds of the present invention include injection, intravenous bolus or infusion, intraperitoneal, subcutaneous, intramuscular, intranasal, and topical, with or without occlusion.

Exemplifying the invention is a pharmaceutical composition comprising any of the compounds described above and a pharmaceutically acceptable carrier. Also exemplifying the invention is a pharmaceutical composition made by combining any of the compounds described above and a pharmaceutically acceptable carrier. An illustration of the invention is a process for making a pharmaceutical composition comprising combining any of the compounds described above and a pharmaceutically acceptable carrier.

The dose may be administered in a single daily dose or the total daily dosage may be administered in divided doses of two, three or four times daily. Furthermore, based on the properties of the individual compound selected for administration, the dose may be administered less frequently, e.g., weekly, twice weekly, monthly, etc. The unit dosage will, of course, be correspondingly larger for the less frequent administration.

When administered via intranasal routes, transdermal routes, by rectal or vaginal suppositories, or through a continual intravenous solution, the dosage administration will, of course, be continuous rather than intermittent throughout the dosage regimen.

The following are examples of representative pharmaceutical dosage forms for the compounds of Formula I:

| Injectable Suspension (I.M.) | mg/mL |
|---|---|
| Compound of Formula I | 10 |
| Methylcellulose | 5.0 |
| Tween 80 | 0.5 |
| Benzyl alcohol | 9.0 |
| Benzalkonium chloride | 1.0 |
| Water for injection to a total volume of 1 ml | |

| Tablet | mg/tablet |
|---|---|
| Compound of Formula I | 25 |
| Microcrystalline Cellulose | 415 |
| Povidone | 14.0 |
| Pregelatinized Starch | 43.5 |
| Magnesium Stearate | 2.5 |
| | 500 |

| Capsule | mg/capsule |
|---|---|
| Compound of Formula I | 25 |
| Lactose Powder | 573.5 |
| Magnesium Stearate | 1.5 |
| | 600 |

| Aerosol | Per canister | |
|---|---|---|
| Compound of Formula I | 24 | mg |
| Lecithin, NF Liq. Conc. | 1.2 | mg |
| Trichlorofluoromethane, NF | 4.025 | g |
| Dichlorodifluoromethane, NF | 12.15 | g |

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I include, but are not limited to: antipsychotic agents, cognition enhancing agents, anti-migraine agents, anti-asthmatic agents, antiinflammatory agents, anxiolytics, anti-Parkinson's agents, anti-epileptics, anorectic agents, serotonin reuptake inhibitors, other anti-obesity agents, as well as antidiabetic agents, lipid lowering agents, and antihypertensive agents which may be administered separately or in the same pharmaceutical compositions.

The present invention also provides a method for the treatment or prevention of a CB1 receptor modulator mediated disease, which method comprises administration to a patient in need of such treatment or at risk of developing a CB1 receptor modulator mediated disease of an amount of a CB1 receptor modulator and an amount of one or more active ingredients, such that together they give effective relief.

In a further aspect of the present invention, there is provided a pharmaceutical composition comprising a CB1 receptor modulator and one or more active ingredients, together with at least one pharmaceutically acceptable carrier or excipient.

Thus, according to a further aspect of the present invention there is provided the use of a CB1 receptor modulator and one or more active ingredients for the manufacture of a medicament for the treatment or prevention of a CB1 receptor modulator mediated disease. In a further or alternative aspect of the present invention, there is therefore provided a product comprising a CB1 receptor modulator and one or more active ingredients as a combined preparation for simultaneous, separate or sequential use in the treatment or prevention of CB1 receptor modulator mediated disease. Such a combined preparation may be, for example, in the form of a twin pack.

It will be appreciated that for the treatment or prevention of eating disorders, including obesity, bulimia nervosa and compulsive eating disorders, a compound of the present invention may be used in conjunction with other anorectic agents.

The present invention also provides a method for the treatment or prevention of eating disorders, which method comprises administration to a patient in need of such treatment an amount of a compound of the present invention and an amount of an anorectic agent, such that together they give effective relief.

Suitable anorectic agents of use in combination with a compound of the present invention include, but are not limited to, aminorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine; and pharmaceutically acceptable salts thereof. A particularly suitable class of anorectic agent are the halogenated amphetamine derivatives, including chlorphentermine, cloforex, clortermine, dexfenfluramine, fenfluramine, picilorex and sibutramine; and pharmaceutically acceptable salts thereof. Particular halogenated amphetamine derivatives of use in combination with a compound of the present invention include: fenfluramine and dexfenfluramine, and pharmaceutically acceptable salts thereof.

The present invention also provides a method for the treatment or prevention of obesity, which method comprises administration to a patient in need of such treatment an amount of a compound of the present invention and an amount of another agent useful in treating obesity and obesity-related conditions, such that together they give effective relief.

Suitable agents of use in combination with a compound of the present invention, include, but are not limited to:

(a) anti-diabetic agents such as (1) PPARγ agonists such as glitazones (e.g. ciglitazone; darglitazone; englitazone; isaglitazone (MCC-555); pioglitazone (ACTOS); rosiglitazone (AVANDIA); troglitazone; rivoglitazone, BRL49653; CLX-0921; 5-BTZD, GW-0207, LG-100641, R483, and LY-300512, and the like and compounds disclosed in WO97/10813, 97/27857, 97/28115, 97/28137, 97/27847, 03/000685, and 03/027112 and SPPARMS (selective PPAR gamma modulators) such as T131 (Amgen), FK614 (Fujisawa), netoglitazone, and metaglidasen; (2) biguanides such as buformin; metformin; and phenformin, and the like; (3) protein tyrosine phosphatase-1B (PTP-1B) inhibitors such as ISIS 113715, A-401674, A-364504, IDD-3, IDD 2846, KP-40046, KR61639, MC52445, MC52453, C7, OC-060062, OC-86839, OC29796, TTP-277BC1, and those agents disclosed in WO 04/041799, 04/050646, 02/26707, 02/26743, 04/092146, 03/048140, 04/089918, 03/002569, 04/065387, 04/127570, and US 2004/167183; (4) sulfonylureas such as acetohexamide; chlorpropamide; diabinese; glibenclamide; glipizide; glyburide; glimepiride; gliclazide; glipentide; gliquidone; glisolamide; tolazamide; and tolbutamide, and the like; (5) meglitinides such as repaglinide, metiglinide (GLUFAST) and nateglinide, and the like; (6) alpha glucoside hydrolase inhibitors such as acarbose; adiposine; camiglibose; emiglitate; miglitol; voglibose; pradimicin-Q; salbostatin; CKD-711; MDL-25,637; MDL-73,945; and MOR 14, and the like; (7) alpha-amylase inhibitors such as tendamistat, trestatin, and Al-3688, and the like; (8) insulin secreatagogues such as linogliride nateglinide, mitiglinide (GLUFAST), ID1101 A-4166, and the like; (9) fatty acid oxidation inhibitors, such as clomoxir, and etomoxir, and the like; (10) A2 antagonists, such as midaglizole; isaglidole; deriglidole; idazoxan; earoxan; and fluparoxan, and the like; (11) insulin or insulin mimetics, such as biota, LP-100, novarapid, insulin detemir, insulin lispro, insulin glargine, insulin zinc suspension (lente and ultralente); Lys-Pro insulin, GLP-1 (17-36), GLP-1 (73-7) (insulintropin); GLP-1 (7-36)-NH₂) exenatide/Exendin-4, Exenatide LAR, Linaglutide, AVE0010, CJC 1131, BIM51077, CS 872, THO318, BAY-694326, GP010, ALBUGON (GLP-1 fused to albumin), HGX-007 (Epac agonist), S-23521, and compounds disclosed in WO 04/022004, WO 04/37859, and the like; (12) non-thiazolidinediones such as JT-501, and farglitazar (GW-2570/GI-262579), and the like; (13) PPARα/γ dual agonists such as AVE 0847, CLX-0940, GW-1536, GW1929, GW-2433, KRP-297, L-796449, LBM 642, LR-90, LY510919, MK-0767, ONO 5129, SB 219994, TAK-559, TAK-654, 677954 (GlaxoSmithkline), E-3030 (Eisai), LY510929 (Lilly), AK109 (Asahi), DRF2655 (Dr. Reddy), DRF8351 (Dr. Reddy), MC3002 (Maxocore), TY51501 (ToaEiyo), naveglitazar, muraglitizar, peliglitazar, tesaglitazar (GALIDA), reglitazar (JTT-501), chiglitazar, and those disclosed in WO 99/16758, WO 99/19313, WO 99/20614, WO 99/38850, WO 00/23415, WO 00/23417, WO 00/23445, WO 00/50414, WO 01/00579, WO 01/79150, WO 02/062799, WO 03/033481, WO 03/033450, WO 03/033453; and (14) other insulin sensitizing drugs; (15) VPAC2 receptor agonists; (16) GLK modulators, such as PSN105, RO 281675, RO 274375 and those disclosed in WO 03/015774, WO 03/000262, WO 03/055482, WO 04/046139, WO 04/045614, WO 04/063179, WO 04/063194, WO 04/050645, and the like; (17) retinoid modulators such as those disclosed in WO 03/000249; (18) GSK 3beta/GSK 3 inhibitors such as 4-[2-(2-bromophenyl)-4-(4-fluorophenyl-1H-imidazol-5-yl]pyridine, CT21022, CT20026, CT-98023, SB-216763, SB410111, SB-675236, CP-70949, XD4241 and those compounds disclosed in WO 03/037869, 03/03877, 03/037891, 03/024447, 05/000192, 05/019218 and the like; (19) glycogen phosphorylase (HGLPa) inhibitors, such as AVE 5688, PSN 357, GPi-879, those disclosed in WO 03/037864, WO 03/091213, WO 04/092158, WO 05/013975, WO 05/013981, US 2004/0220229, and JP 2004-196702, and the like; (20) ATP consumption promotors such as those disclosed in WO 03/007990; (21) fixed combinations of PPAR γ agonists and metformin such as AVANDAMET; (22) PPAR pan agonists such as GSK 677954; (23) GPR40 (G-protein coupled receptor 40) also called SNORF 55 such as BG 700, and those disclosed in WO 04/041266, 04/022551, 03/099793; (24) GPR119 (also called RUP3; SNORF 25) such as RUP3, HGPRBMY26, PFI 007, SNORF 25; (25) adenosine receptor 2B antagonists such as ATL-618, ATI-802, E3080, and the like; (26) carnitine palmitoyl transferase inhibitors such as ST 1327, and ST 1326, and the like; (27) Fructose 1,6-bisphosphohatase inhibitors such as CS-917, MB7803, and the like; (28) glucagon antagonists such as AT77077, BAY 694326, GW 4123X, NN2501, and those disclosed in WO 03/064404, WO 05/00781, US 2004/0209928, US 2004/029943, and the like; (30) glucose-6-phosphase inhibitors; (31) phosphoenolpyruvate carboxykinase (PEPCK) inhibitors; (32) pyruvate dehydrogenase kinase (PDK) activators; (33) RXR agonists such as MC1036, CS00018, JNJ 10166806, and those disclosed in WO 04/089916, U.S. Pat. No. 6,759,546, and the like; (34) SGLT inhibitors such as AVE 2268, KGT 1251, T1095/RWJ 394718; (35) BLX-1002;

(b) lipid lowering agents such as (1) bile acid sequestrants such as, cholestyramine, colesevelem, colestipol, dialkylaminoalkyl derivatives of a cross-linked dextran; Colestid®; LoCholest®; and Questran®, and the like; (2) HMG-CoA reductase inhibitors such as atorvastatin, itavastatin, pitavastatin, fluvastatin, lovastatin, pravastatin, rivastatin, rosuvastatin, simvastatin, rosuvastatin (ZD-4522), and the like, particularly simvastatin; (3) HMG-CoA synthase inhibitors; (4) cholesterol absorption inhibitors such as FMVP4 (Forbes Medi-Tech), KT6-971 (Kotobuki Pharmaceutical), FM-VA12 (Forbes Medi-Tech), FM-VP-24 (Forbes Medi-Tech), stanol esters, beta-sitosterol, sterol glycosides such as tiqueside; and azetidinones such as ezetimibe, and those disclosed in WO 04/005247 and the like; (5) acyl coenzyme A-cholesterol acyl transferase (ACAT) inhibitors such as avasimibe, eflucimibe, pactimibe (KY505), SMP 797 (Sumitomo), SM32504 (Sumitomo), and those disclosed in WO 03/091216, and the like; (6) CETP inhibitors such as JTT 705 (Japan Tobacco), torcetrapib, CP 532,632, BAY63-2149 (Bayer), SC 591, SC 795, and the like; (7) squalene synthetase inhibitors; (8) anti-oxidants such as probucol, and the like; (9) PPARα agonists such as beclofibrate, benzafibrate, ciprofibrate, clofibrate, etofibrate, fenofibrate, gemcabene, and gemfibrozil, GW 7647, BM 170744 (Kowa), LY518674 (Lilly), GW590735 (GlaxoSmithkline), KRP-101 (Kyorin), DRF10945 (Dr. Reddy), NS-220/R1593 (Nippon Shinyaku/Roche, ST1929 (Sigma Tau) MC3001/MC3004 (MaxoCore Pharmaceuticals, gemcabene calcium, other fibric acid derivatives, such as Atromid®, Lopid® and Tricor®, and those disclosed in U.S. Pat. No. 6,548,538, and the like; (10) FXR receptor modulators such as GW 4064 (GlaxoSmithkline), SR 103912, QRX401, LN-6691 (Lion Bioscience), and those disclosed in WO 02/064125, WO 04/045511, and the like; (11) LXR receptor modulators such as GW 3965 (GlaxoSmithkline), T9013137, and XTCO179628 (X-Ceptor Therapeutics/Sanyo), and those disclosed in WO 03/031408, WO 03/063796, WO 04/072041, and the like; (12) lipoprotein synthesis inhibitors such as niacin; (13) renin angiotensin system inhibitors; (14) PPAR δ partial agonists, such as those disclosed in WO 03/024395; (15) bile acid reabsorption inhibitors, such as BARI 11453, SC435, PHA384640, S8921, AZD7706, and the like; and bile acid sequesterants such as colesevelam (WELCHOL/CHOLESTAGEL), (16) PPARδ agonists such as GW 501516 (Ligand, GSK), GW 590735, GW-0742 (GlaxoSmithkline), T659 (Amgen/Tularik), LY934 (Lilly), NNC610050 (Novo Nordisk) and those disclosed in WO97/28149, WO 01/79197, WO 02/14291, WO 02/46154, WO 02/46176, WO 02/076957, WO 03/016291, WO 03/033493, WO 03/035603, WO 03/072100, WO 03/097607, WO 04/005253, WO 04/007439, and JP10237049, and the like; (17) triglyceride synthesis inhibitors; (18) microsomal triglyceride transport (MTTP) inhibitors, such as implitapide, LAB687, JTT130 (Japan Tobacco), CP346086, and those disclosed in WO 03/072532, and the like; (19) transcription modulators; (20) squalene epoxidase inhibitors; (21) low density lipoprotein (LDL) receptor inducers; (22) platelet aggregation inhibitors; (23) 5-LO or FLAP inhibitors; and (24) niacin receptor agonists including HM74A receptor agonists; (25) PPAR modulators such as those disclosed in WO 01/25181, WO 01/79150, WO 02/79162, WO 02/081428, WO 03/016265, WO 03/033453; (26) niacin-bound chromium, as disclosed in WO 03/039535; (27) substituted acid derivatives disclosed in WO 03/040114; (28) infused HDL such as LUV/ETC-588 (Pfizer), APO-A1 Milano/ETC216 (Pfizer), ETC-642 (Pfizer), ISIS301012, D4F (Bruin Pharma), synthetic trimeric ApoA1, Bioral Apo A1 targeted to foam cells, and the like; (29) IBAT inhibitors such as BARI143/HMR145A/HMR1453 (Sanofi-Aventis, PHA384640E (Pfizer), S8921 (Shionogi) AZD7806 (AstrZeneca), AK105 (Asah Kasei), and the like; (30) Lp-PLA2 inhibitors such as SB480848 (GlaxoSmithkline), 659032 (GlaxoSmithkline), 677116 (GlaxoSmithkline), and the like; (31) other agents which affect lipic composition including ETC1001/ESP31015 (Pfizer), ESP-55016 (Pfizer), AGI1067 (AtheroGenics), AC3056 (Amylin), AZD4619 (AstrZeneca); and

(c) anti-hypertensive agents such as (1) diuretics, such as thiazides, including chlorthalidone, chlorthiazide, dichlorophenamide, hydroflumethiazide, indapamide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like; (2) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (3) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (4) angiotensin converting enzyme (ACE) inhibitors such as benazepril; captopril; cilazapril; delapril; enalapril; fosinopril; imidapril; losinopril; moexipril; quinapril; quinaprilat; ramipril; perindopril; perindropril; quanipril; spirapril; tenocapril; trandolapril, and zofenopril, and the like; (5) neutral endopeptidase inhibitors such as omapatrilat, cadoxatril and ecadotril, fosidotril, sampatrilat, AVE7688, ER4030, and the like; (6) endothelin antagonists such as tezosentan, A308165, and YM62899, and the like; (7) vasodilators such as hydralazine, clonidine, minoxidil, and nicotinyl alcohol, and the like; (8) angiotensin II receptor antagonists such as candesartan, eprosartan, irbesartan, losartan, pratosartan, tasosartan, telmisartan, valsartan, and EXP-3137, FI6828K, and RNH6270, and the like; (9) α/β adrenergic blockers as nipradilol, arotinolol and amosulalol, and the like; (10) alpha 1 blockers, such as terazosin, urapidil, prazosin, bunazosin, trimazosin, doxazosin, naftopidil, indoramin, WHIP 164, and XEN010, and the like; (11) alpha 2 agonists such as lofexidine, tiamenidine, moxonidine, rilmenidine and guanobenz, and the like; (12) aldosterone inhibitors, and the like; (13) angiopoietin-2-binding agents such as those disclosed in WO 03/030833; and

(d) anti-obesity agents, such as (1) 5HT (serotonin) transporter inhibitors, such as paroxetine, fluoxetine, fenfluramine, fluvoxamine, sertraline, and imipramine, and those disclosed in WO 03/00663, as well as serotonin/noradrenaline re uptake inhibitors such as sibutramine (MERIDIA/REDUCTIL) and dopamine uptake inhibitor/Norepenephrine uptake inhibitors such as radafaxine hydrochloride, 353162 (GlaxoSmithkline), and the like; (2) NE (norepinephrine) transporter inhibitors, such as GW 320659, despiramine, talsupram, and nomifensine; (3) CB1 (cannabinoid-1 receptor) antagonist/inverse agonists, such as rimonabant (ACCOMPLIA Sanofi Synthelabo), SR-147778 (Sanofi Synthelabo), AVE1625 (Sanofi-Aventis), BAY 65-2520 (Bayer), SLV 319 (Solvay), SLV326 (Solvay), CP945598 (Pfizer), E-6776 (Esteve), O1691 (Organix), ORG14481 (Organon), VER24343 (Vernalis), NESS0327 (Univ of Sassari/Univ of Cagliari), and those disclosed in U.S. Pat. Nos. 4,973,587, 5,013,837, 5,081,122, 5,112,820, 5,292,736, 5,532,237, 5,624,941, 6,028,084, and 6,509,367; and WO 96/33159, WO97/29079, WO98/31227, WO 98/33765, WO98/37061, WO98/41519, WO98/43635, WO98/43636, WO99/02499, WO00/10967, WO00/10968, WO 01/09120, WO 01/58869, WO 01/64632, WO 01/64633, WO 01/64634, WO 01/70700, WO 01/96330, WO 02/076949, WO 03/006007, WO 03/007887, WO 03/020217, WO 03/026647, WO 03/026648, WO 03/027069, WO 03/027076, WO 03/027114, WO 03/037332, WO 03/040107, WO 04/096763, WO 04/111039, WO 04/111033, WO 04/111034, WO 04/111038, WO 04/013120, WO 05/000301, WO 05/016286, WO 05/066126 and EP-658546 and the like; (4) ghrelin agonists/antagonists, such as BVT81-97 (BioVitrum), RC1291 (Rejuvenon), SRD-04677 (Sumitomo), unacylated ghrelin (TheraTechnologies), and those disclosed in WO 01/87335, WO 02/08250, WO 05/012331, and the like; (5) H3 (histamine H3) antagonist/inverse agonists, such as thioperamide, 3-(1H-imidazol-4-yl)propyl N-(4-pentenyl)carbamate), clobenpropit, iodophenpropit, imoproxifan, GT2394 (Gliatech), and A331440, and those disclosed in WO 02/15905; and O-[3-(1H-imidazol-4-yl)propanol]carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm. (Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)) and histamine H3 receptor modulators such as those disclosed in WO 03/024928 and WO 03/024929; (6) melanin-concentrating hormone 1 receptor (MCH1R) antagonists, such as T-226296 (Takeda), T71 (Takeda/Amgen), AMGN-608450, AMGN-503796 (Amgen), 856464 (GlaxoSmithkline), A224940 (Abbott), A798 (Abbott), ATC0175/AR224349 (Arena Pharmaceuticals), GW803430 (GlaxoSmithkline), NBI-1A (Neurocrine Biosciences), NGX-1 (Neurogen), SNP-7941 (Synaptic), SNAP9847 (Synaptic), T-226293 (Schering Plough), TPI-1361-17 (Saitama Medical School/University of California Irvine), and those disclosed WO 01/21169, WO 01/82925, WO 01/87834, WO 02/051809, WO 02/06245, WO 02/076929, WO 02/076947, WO 02/04433, WO 02/51809, WO 02/083134, WO 02/094799, WO 03/004027, WO 03/13574, WO 03/15769, WO 03/028641, WO 03/035624, WO 03/033476, WO 03/033480, WO 04/004611, WO 04/004726, WO 04/011438, WO 04/028459, WO 04/034702, WO 04/039764, WO 04/052848, WO 04/087680; and Japanese Patent Application Nos. JP 13226269, JP 1437059, JP2004315511, and the like; (7) MCH2R (melanin concentrating hormone 2R) agonist/antagonists; (8) NPY1 (neuropeptide Y Y1) antagonists, such as BMS205749, BIBP3226, J-115814, BIBO 3304, LY-357897, CP-671906, and GI-264879A; and those disclosed in U.S. Pat. No. 6,001,836; and WO 96/14307, WO 01/23387, WO 99/51600, WO 01/85690, WO 01/85098, WO 01/85173, and WO 01/89528; (9) NPY5 (neuropeptide Y Y5) antagonists, such as 152,804, S2367 (Shionogi), E-6999 (Esteve), GW-569180A, GW-594884A (GlaxoSmithkline), GW-587081X, GW-548118X; FR 235,208; FR226928, FR 240662, FR252384; 1229U91, GI-264879A, CGP71683A, C-75 (Fasgen) LY-377897, LY366377, PD-160170, SR-120562A, SR-120819A,S2367 (Shionogi), JCF-104, and H409/22; and those compounds disclosed in U.S. Pat. Nos. 6,140,354, 6,191,160, 6,258,837, 6,313,298, 6,326,375, 6,329,395, 6,335,345, 6,337,332, 6,329,395, and 6,340,683; and EP-01010691, EP-01044970, and FR252384; and PCT Publication Nos. WO 97/19682, WO 97/20820, WO 97/20821, WO 97/20822, WO 97/20823, WO 98/27063, WO 00/107409, WO 00/185714, WO 00/185730, WO 00/64880, WO 00/68197, WO 00/69849, WO 01/09120, WO 01/14376, WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/20488, WO 02/22592, WO 02/48152, WO 02/49648, WO 02/051806, WO 02/094789, WO 03/009845, WO 03/014083, WO 03/022849, WO 03/028726, WO 05/014592, WO 05/01493; and Norman et al., J. Med. Chem. 43:4288-4312 (2000); (10) leptin, such as recombinant human leptin (PEG-OB, Hoffman La Roche) and recombinant methionyl human leptin (Amgen); (11) leptin derivatives, such as those disclosed in U.S. Pat. Nos. 5,552,524; 5,552,523; 5,552,522; 5,521,283; and WO 96/23513; WO 96/23514; WO 96/23515; WO 96/23516; WO 96/23517; WO 96/23518; WO 96/23519; and WO 96/23520; (12) opioid antagonists, such as nalmefene (Revex®), 3-methoxynaltrexone, naloxone, and naltrexone; and those disclosed in WO 00/21509; (13) orexin antagonists, such as SB-334867-A (GlaxoSmithkline); and those disclosed in WO 01/96302, 01/68609, 02/44172, 02/51232, 02/51838, 02/089800, 02/090355, 03/023561, 03/032991, 03/037847, 04/004733, 04/026866, 04/041791, 04/085403, and the like; (14) BRS3 (bombesin receptor subtype 3) agonists; (15) CCK-A (cholecystokinin-A) agonists, such as AR-R 15849, GI 181771, JMV-180, A-71378, A-71623, PD170292, PD 149164, SR146131, SR125180, butabindide, and those disclosed in U.S. Pat. No. 5,739,106; (16) CNTF (ciliary neurotrophic factors), such as GI-181771 (Glaxo-SmithKline); SR146131 (Sanofi Synthelabo); butabindide; and PD170,292, PD 149164 (Pfizer); (17) CNTF derivatives, such as axokine (Regeneron); and those disclosed in WO 94/09134, WO 98/22128, and WO 99/43813; (18) GHS (growth hormone secretagogue receptor) agonists, such as NN703, hexarelin, MK-0677, SM-130686, CP-424,391, L-692,429 and L-163,255, and those disclosed in U.S. Pat. No. 6,358,951, U.S. Patent Application Nos. 2002/049196 and 2002/022637; and WO 01/56592, and WO 02/32888; (19) 5HT2c (serotonin receptor 2c) agonists, such as APD3546/AR10A (Arena Pharmaceuticals), ATH88651 (Athersys), ATH88740 (Athersys), BVT933 (Biovitrum/GSK), DPCA37215 (BMS), IK264; LY448100 (Lilly), PNU 22394; WAY 470 (Wyeth), WAY629 (Wyeth), WAY161503 (Biovitrum), R-1065, VR1065 (Vernalis/Roche) YM 348; and those disclosed in U.S. Pat. No. 3,914,250; and PCT Publications 01/66548, 02/36596, 02/48124, 02/10169, 02/44152; 02/51844, 02/40456, 02/40457, 03/057698, 05/000849, and the like; (20) Mc3r (melanocortin 3 receptor) agonists; (21) Mc4r (melanocortin 4 receptor) agonists, such as CHIR86036 (Chiron), CHIR915 (Chiron); ME-10142 (Melacure), ME-10145 (Melacure), HS-131 (Melacure), NBI72432 (Neurocrine Biosciences), NNC 70-619 (Novo Nordisk), TTP2435 (Transtech) and those disclosed in PCT Publications WO 99/64002, 00/74679, 01/991752, 01/0125192, 01/52880, 01/74844, 01/70708, 01/70337, 01/91752, 01/010842, 02/059095, 02/059107, 02/059108, 02/059117, 02/062766, 02/069095, 02/12166, 02/11715, 02/12178, 02/15909, 02/38544, 02/068387, 02/068388, 02/067869, 02/081430, 03/06604, 03/007949, 03/009847, 03/009850, 03/013509, 03/031410, 03/094918, 04/028453, 04/048345, 04/050610, 04/075823, 04/083208, 04/089951, 05/000339, and EP 1460069, and US 2005049269, and JP2005042839, and the like; (22) monoamine reuptake inhibitors, such as sibutratmine (Meridia®/Reductil®) and salts thereof, and those compounds disclosed in U.S. Pat. Nos. 4,746,680, 4,806,570, and 5,436,272, and U.S. Patent Publication No. 2002/0006964, and WO 01/27068, and WO 01/62341; (23) serotonin reuptake inhibitors, such as dexfenfluramine, fluoxetine, and those in U.S. Pat. No. 6,365,633, and WO 01/27060, and WO 01/162341; (24) GLP-1 (glucagon-like peptide 1) agonists; (25) Topiramate (Topimax®); (26) phytopharm compound 57 (CP 644,673); (27) ACC2 (acetyl-CoA carboxylase-2) inhibitors; (28) β3 (beta adrenergic receptor 3) agonists, such as rafebergron/AD9677/TAK677 (Dainippon/Takeda), CL-316,243, SB 418790, BRL-37344, L-796568, BMS-196085, BRL-35135A, CGP12177A, BTA-243, GRC1087 (Glenmark Pharmaceuticals) GW 427353 (solabegron hydrochloride), Trecadrine, Zeneca D7114, N-5984 (Nisshin Kyorin), LY-377604 (Lilly), KT07924 (Kissei), SR 59119A, and those disclosed in U.S. Pat. No. 5,705,515, U.S. Pat. No. 5,451,677; and WO94/18161, WO95/29159, WO97/46556, WO98/04526 WO98/32753, WO 01/74782, WO 02/32897, WO 03/014113, WO 03/016276, WO 03/016307, WO 03/024948, WO 03/024953, WO 03/037881, WO 04/108674, and the like; (29) DGAT1 (diacylglycerol acyltransferase 1) inhibitors; (30) DGAT2 (diacylglycerol acyltransferase 2) inhibitors; (31) FAS (fatty acid synthase) inhibitors, such as Cerulenin and C75; (32) PDE (phosphodiesterase) inhibitors, such as theophylline, pentoxifylline, zaprinast, sildenafil, amrinone, milrinone, cilostamide, rolipram, and cilomilast, as well as those described in WO 03/037432, WO 03/037899; (33) thyroid hormone β agonists, such as KB-2611 (KaroBioBMS), and those disclosed in WO 02/15845; and Japanese Patent Application No. JP 2000256190; (34) UCP-1 (uncoupling protein 1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), and retinoic acid; and those disclosed in WO 99/00123; (35) acyl-estrogens, such as oleoyl-estrone, disclosed in del Mar-Grasa, M. et al., Obesity Research, 9:202-9 (2001); (36) glucocorticoid receptor antagonists, such as CP472555 (Pfizer), KB 3305, and those disclosed in WO 04/000869, WO 04/075864, and the like; (37) 11β HSD-1 (11-beta hydroxy steroid dehydrogenase type 1) inhibitors, such as BVT 3498 (AMG 331), BVT 2733, 3-(1-adamantyl)-4-ethyl-5-(ethylthio)-4H-1,2,4-triazole, 3-(1-adamantyl)-5-(3,4,5-trimethoxyphenyl)-4-methyl-4H-1,2,4-triazole, 3-adamantanyl-4,5,6,7,8,9,10,11,12,3a-decahydro-1,2,4-triazolo[4,3-a][11]annulene, and those compounds disclosed in WO 01/90091, 01/90090, 01/90092, 02/072084, 04/011410, 04/033427, 04/041264, 04/027047, 04/056744, 04/065351, 04/089415, 04/037251, and the like; (38) SCD-1 (stearoyl-CoA desaturase-1) inhibitors; (39) dipeptidyl peptidase IV (DPP-4) inhibitors, such as isoleucine thiazolidide, valine pyrrolidide, sitagliptin, saxagliptin, NVP-DPP728, LAF237 (vildagliptin), P93/01, TSL 225, TMC-2A/2B/2C, FE 999011, P9310/K364, VIP 0177, SDZ 274-444, GSK 823093, E 3024, SYR 322, TS021, SSR 162369, GRC 8200, K579, NN7201, CR 14023, PHX 1004, PHX 1149, PT-630, SK-0403; and the compounds disclosed in WO 02/083128, WO 02/062764, WO 02/14271, WO 03/000180, WO 03/000181, WO 03/000250, WO 03/002530, WO 03/002531, WO 03/002553, WO 03/002593, WO 03/004498, WO 03/004496, WO 03/005766, WO 03/017936, WO 03/024942, WO 03/024965, WO 03/033524, WO 03/055881, WO 03/057144, WO 03/037327, WO 04/041795, WO 04/071454, WO 04/0214870, WO 04/041273, WO 04/041820, WO 04/050658, WO 04/046106, WO 04/067509, WO 04/048532, WO 04/099185, WO 04/108730, WO 05/009956, WO 04/09806, WO 05/023762, US 2005/043292, and EP 1 258 476; (40) lipase inhibitors, such as tetrahydrolipstatin (orlistat/XENICAL), ATL962 (Alizyme/Takeda), GT389255 (Genzyme/Peptimmune) Triton WR1339, RHC80267, lipstatin, teasaponin, and diethylumbelliferyl phosphate, FL-386, WAY-121898, Bay-N-3176, valilactone, esteracin, ebelactone A, ebelactone B, and RHC 80267, and those disclosed in WO 01/77094, WO 04/111004, and U.S. Pat. Nos. 4,598,089, 4,452,813, 5,512,565, 5,391,571, 5,602,151, 4,405,644, 4,189,438, and 4,242,453, and the like; (41) fatty acid transporter inhibitors; (42) dicarboxylate transporter inhibitors; (43) glucose transporter inhibitors; and (44) phosphate transporter inhibitors; (45) anorectic bicyclic compounds such as 1426 (Aventis) and 1954 (Aventis), and the compounds disclosed in WO 00/18749, WO 01/32638, WO 01/62746, WO 01/62747, and WO 03/015769; (46) peptide YY and PYY agonists such as PYY336 (Nastech/Merck), AC162352 (IC Innovations/Curis/Amylin), TM30335/TM30338 (7TM Pharma), PYY336 (Emisphere Technologies), pegylated peptide YY3-36, those disclosed in WO 03/026591, 04/089279, and the like; (47) lipid metabolism modulators such as maslinic acid, erythrodiol, ursolic acid uvaol, betulinic acid, betulin, and the like and compounds disclosed in WO 03/011267; (48) transcription factor modulators such as those disclosed in WO 03/026576; (49) Mc5r (melanocortin 5 receptor) modulators, such as those disclosed in WO 97/19952, WO 00/15826, WO 00/15790, US 20030092041, and the like; (50) Brain derived neutotropic factor (BDNF), (51) Mc1r (melanocortin 1 receptor modulators such as LK-184 (Proctor & Gamble), and the like; (52) 5HT6 antagonists such as BVT74316 (BioVitrum), BVT5182c (BioVitrum), E-6795 (Esteve), E-6814 (Esteve), SB399885 (GlaxoSmithkline), SB271046 (GlaxoSmithkline), RO-046790 (Roche), and the like; (53) fatty acid transport protein 4 (FATP4); (54) acetyl-CoA carboxylase (ACC) inhibitors such as CP640186, CP610431, CP640188 (Pfizer); (55) C-terminal growth hormone fragments such as AOD9604 (Monash Univ/Metabolic Pharmaceuticals), and the like; (56) oxyntomodulin; (57) neuropeptide FF receptor antagonists such as those disclosed in WO 04/083218, and the like; (58) amylin agonists such as Symlin/pramlintide/AC137 (Amylin); (59) Hoodia and trichocaulon extracts; (60) BVT74713 and other gut lipid appetite suppressants; (61) dopamine agonists such as bupropion (WELLBUTRIN/GlaxoSmithkline); (62) zonisamide (ZONEGRAN/Dainippon/Elan), and the like.

Specific compounds of use in combination with a compound of the present invention include: simvastatin, mevastatin, ezetimibe, atorvastatin, sitagliptin, metformin, sibutramine, orlistat, Qnexa, topiramate, naltrexone, bupriopion, phentermine, and losartan, losartan with hydrochlorothiazide. Specific CB1 antagonists/inverse agonists of use in combination with a compound of the present invention include: those described in WO03/077847, including: N-[3-(4-chlorophenyl)-2(S)-phenyl-1(S)-methylpropyl]-2-(4-trifluoromethyl-2-pyrimidyloxy)-2-methylpropanamide, N-[3-(4-chlorophenyl)-2-(3-cyanophenyl)-1-methylpropyl]-2-(5-trifluoromethyl-2-pyridyloxy)-2-methylpropanamide, N-[3-(4-chlorophenyl)-2-(5-chloro-3-pyridyl)-1-methylpropyl]-2-(5-trifluoromethyl-2-pyridyloxy)-2-methylpropanamide, and pharmaceutically acceptable salts thereof; as well as those in WO05/000809, which includes the following: 3-{1-[bis(4-chlorophenyl)methyl]azetidin-3-ylidene}-3-(3,5-difluorophenyl)-2,2-dimethylpropanenitrile, 1-{1-[1-(4-chlorophenyl)pentyl]azetidin-3-yl}-1-(3,5-difluorophenyl)-2-methylpropan-2-ol. 3-((S)-(4-chlorophenyl) {3-[(1S)-1-(3,5-difluorophenyl)-2-hydroxy-2-methylpropyl]azetidin-1-yl}methyl)benzonitrile, 3-((S)-(4-chlorophenyl) {3-[(1S)-1-(3,5-difluorophenyl)-2-fluoro-2-methylpropyl]azetidin-1-yl}methyl)benzonitrile, 3-((4-chlorophenyl){3-[1-(3,5-difluorophenyl)-2,2-dimethylpropyl]azetidin-1-yl}methyl)benzonitrile, 3-((1S)-1-{1-[(S)-(3-cyanophenyl)(4-cyanophenyl)methyl]azetidin-3-yl}-2-fluoro-2-methylpropyl)-5-fluorobenzonitrile, 3-[(S)-(4-chlorophenyl)(3-{(1S)-2-fluoro-1-[3-fluoro-5-(4H-1,2,4-triazol-4-yl)phenyl]-2-methylpropyl}azetidin-1-yl)methyl]benzonitrile, and 5-((4-chlorophenyl) {3-[(1S)-1-(3,5-difluorophenyl)-2-fluoro-2-methylpropyl]azetidin-1-yl}methyl)thiophene-3-carbonitrile, and pharmaceutically acceptable salts thereof; as well as: 3-[(S)-(4-chlorophenyl)(3-{(1S)-2-fluoro-1-[3-fluoro-5-(5-oxo-4,5-dihydro-1,3,4-oxadiazol-2-yl)phenyl]-2-methylpropyl}azetidin-1-yl)methyl]benzonitrile, 3-[(S)-(4-chlorophenyl)(3-{(1S)-2-fluoro-1-[3-fluoro-5-(1,3,4-oxadiazol-2-yl)phenyl]-2-methylpropyl}azetidin-1-yl)methyl]benzonitrile, 3-[(S)-(3-{(1S)-1-[3-(5-amino-1,3,4-oxadiazol-2-yl)-5-fluorophenyl]-2-fluoro-2-methylpropyl}azetidin-1-yl)(4-chlorophenyl)methyl]benzonitrile, 3-[(S)-(4-cyanophenyl)(3-{(1S)-2-fluoro-1-[3-fluoro-5-(5-oxo-4,5-dihydro-1,3,4-oxadiazol-2-yl)phenyl]-2-methylpropyl}azetidin-1-yl)methyl]benzonitrile, 3-[(S)-(3-{(1S)-1-[3-(5-amino-1,3,4-oxadiazol-2-yl)-5-fluorophenyl]-2-fluoro-2-methylpropyl}azetidin-1-yl)(4-cyanophenyl)methyl]benzonitrile, 3-[(S)-(4-cyanophenyl)(3-{(1S)-2-fluoro-1-[3-fluoro-5-(1,3,4-oxadiazol-2-yl)phenyl]-2-methylpropyl}azetidin-1-yl)methyl]benzonitrile, 3-[(S)-(4-chlorophenyl)(3-{(1S)-2-fluoro-1-[3-fluoro-5-(1,2,4-oxadiazol-3-yl)phenyl]-2-methylpropyl}azetidin-1-yl)methyl]benzonitrile, 3-[(1S)-1-(1-{(S)-(4-cyanophenyl) [3-(1,2,4-oxadiazol-3-yl)phenyl]-methyl}azetidin-3-yl)-2-fluoro-2-methylpropyl]-5-fluorobenzonitrile, 5-(3-{1-[1-(diphenylmethyl)azetidin-3-yl]-2-fluoro-2-methylpropyl}-5-fluorophenyl)-1H-tetrazole, 5-(3-{1-[1-(diphenylmethyl)azetidin-3-yl]-2-fluoro-2-methylpropyl}-5-fluorophenyl)-1-methyl-1H-tetrazole, 5-(3-{1-[1-(diphenylmethyl)azetidin-3-yl]-2-fluoro-2-methylpropyl}-5-fluorophenyl)-2-methyl-2H-tetrazole, 3-[(4-chlorophenyl)(3-{2-fluoro-1-[3-fluoro-5-(2-methyl-2H-tetrazol-5-yl)phenyl]-2-methylpropyl}azetidin-1-yl)methyl]benzonitrile, 3-[(4-chlorophenyl)(3-{2-fluoro-1-[3-fluoro-5-(1-methyl-1H-tetrazol-5-yl)phenyl]-2-methylpropyl}azetidin-1-yl)methyl]benzonitrile, 3-[(4-cyanophenyl)(3-{2-fluoro-1-[3-fluoro-5-(1-methyl-1H-tetrazol-5-yl)phenyl]-2-methylpropyl}azetidin-1-yl)methyl]benzonitrile, 3-[(4-cyanophenyl)(3-{2-fluoro-1-[3-fluoro-5-(2-methyl-2H-tetrazol-5-yl)phenyl]-2-methylpropyl}azetidin-1-yl)methyl]benzonitrile, 5-{3-[(S)-{3-[(1S)-1-(3-bromo-5-fluorophenyl)-2-fluoro-2-methylpropyl]azetidin-1-yl}(4-chlorophenyl)methyl]phenyl}-1,3,4-oxadiazol-2(3H)-one, 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, 3-[(1S)-1-(1-{(S)-(4-cyanophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, 3-[(1S)-1-(1-{(S)-(4-cyanophenyl)[3-(1,3,4-oxadiazol-2-yl)phenyl]methyl}azetidin-3-yl)-2-fluoro-2-methylpropyl]-5-fluorobenzonitrile, 3-[(1S)-1-(1-{(S)-(4-chlorophenyl)[3-(1,3,4-oxadiazol-2-yl)phenyl]methyl}azetidin-3-yl)-2-fluoro-2-methylpropyl]-5-fluorobenzonitrile, 3-((1S)-1-{1-[(S)-[3-(5-amino-1,3,4-oxadiazol-2-yl)phenyl](4-chlorophenyl)methyl]azetidin-3-yl}-2-fluoro-2-methylpropyl)-5-fluorobenzonitrile, 3-((1S)-1-{1-[(S)-[3-(5-amino-1,3,4-oxadiazol-2-yl)phenyl](4-cyanophenyl)methyl]azetidin-3-yl}-2-fluoro-2-methylpropyl)-5-fluorobenzonitrile, 3-[(1S)-1-(1-{(S)-(4-cyanophenyl)[3-(1,2,4-oxadiazol-3-yl)phenyl]methyl}azetidin-3-yl)-2-fluoro-2-methylpropyl]-5-fluorobenzonitrile, 3-[(1S)-1-(1-{(S)-(4-chlorophenyl)[3-(1,2,4-oxadiazol-3-yl)phenyl]methyl}azetidin-3-yl)-2-fluoro-2-methylpropyl]-5-fluorobenzonitrile, 5-[3-((S)-(4-chlorophenyl) {3-[(1S)-1-(3,5-difluorophenyl)-2-fluoro-2-methylpropyl]azetidin-1-yl}methyl)phenyl]-1,3,4-oxadiazol-2(3H)-one, 5-[3-((S)-(4-chlorophenyl) {3-[(1S)-1-(3,5-difluorophenyl)-2-fluoro-2-methylpropyl]azetidin-1-yl}methyl)phenyl]-1,3,4-oxadiazol-2(3H)-one, 4-{(S)-{3-[(1S)-1-(3,5-difluorophenyl)-2-fluoro-2-methylpropyl]azetidin-1-yl}[3-(5-oxo-4,5-dihydro-1,3,4-oxadiazol-2-yl)phenyl]methyl}-benzonitrile, and pharmaceutically acceptable salts thereof.

Specific NPY5 antagonists of use in combination with a compound of the present invention include: 3-oxo-N-(5-phenyl-2-pyrazinyl)-spiro[isobenzofuran-1(3H), 4′-piperidine]-1′-carboxamide, 3-oxo-N-(7-trifluoromethylpyrido[3,2-b]pyridin-2-yl)spiro-[isobenzofuran-1(3H), 4′-piperidine]-1′-carboxamide, N-[5-(3-fluorophenyl)-2-pyrimidinyl]-3-oxospiro-[isobenzofuran-1(3H), 4′-piperidine]-1′-carboxamide, trans-3′-oxo-N-(5-phenyl-2-pyrimidinyl)spiro[cyclohexane-1,1′(3′H)-isobenzofuran]-4-carboxamide, trans-3′-oxo-N-[1-(3-quinolyl)-4-imidazolyl]spiro[cyclohexane-1,1′(3′H)-isobenzofuran]-4-carboxamide, trans-3-oxo-N-(5-phenyl-2-pyrazinyl)spiro[4-azaiso-benzofuran-1(3H), 1′-cyclohexane]-4′-carboxamide, trans-N-[5-(3-fluorophenyl)-2-pyrimidinyl]-3-oxospiro[5-azaisobenzofuran-1(3H), 1′-cyclohexane]-4′-carboxamide, trans-N-[5-(2-fluorophenyl)-2-pyrimidinyl]-3-oxospiro[5-azaisobenzofuran-1(3H), 1′-cyclohexane]-4′-carboxamide, trans-N-[1-(3,5-difluorophenyl)-4-imidazolyl]-3-oxospiro[7-azaisobenzofuran-1(3H), 1′-cyclohexane]-4′-carboxamide, trans-3-oxo-N-(1-phenyl-4-pyrazolyl)spiro[4-azaisobenzofuran-1(3H), 1′-cyclohexane]-4′-carboxamide, trans-N-[1-(2-fluorophenyl)-3-pyrazolyl]-3-oxospiro[6-azaisobenzofuran-1(3H), 1′-cyclohexane]-4′-carboxamide, trans-3-oxo-N-(1-phenyl-3-pyrazolyl)spiro[6-azaisobenzofuran-1(3H), 1′-cyclohexane]-4′-carboxamide, trans-3-oxo-N-(2-phenyl-1,2,3-triazol-4-yl)spiro[6-azaisobenzofuran-1(3H), 1′-cyclohexane]-4′-carboxamide, and pharmaceutically acceptable salts and esters thereof.

Specific ACC-1/2 inhibitors of use in combination with a compound of the present invention include: 1′-[(4,8-dimethoxyquinolin-2-yl)carbonyl]-6-(1H-tetrazol-5-yl)spiro[chroman-2,4′-piperidin]-4-one; (5-{1′-[(4,8-dimethoxyquinolin-2-yl)carbonyl]-4-oxospiro[chroman-2,4′-piperidin]-6-yl}-2H-tetrazol-2-yl)methyl pivalate; 5-{1′-[(8-cyclopropyl-4-methoxyquinolin-2-yl)carbonyl]-4-oxospiro[chroman-2,4′-piperidin]-6-yl}nicotinic acid; 1′-(8-methoxy-4-morpholin-4-yl-2-naphthoyl)-6-(1H-tetrazol-5-yl)spiro[chroman-2,4′-piperidin]-4-one; and 1′-[(4-ethoxy-8-ethylquinolin-2-yl)carbonyl]-6-(1H-tetrazol-5-yl)spiro[chroman-2,4′-piperidin]-4-one; and pharmaceutically acceptable salts and esters thereof.

Specific MCH1R antagonist compounds of use in combination with a compound of the present invention include: 1-{4-[(1-ethylazetidin-3-yl)oxy]phenyl}-4-[(4-fluorobenzyl)oxy]pyridin-2(1H)-one, 4-[(4-fluorobenzyl)oxy]-1-{4-[(1-isopropylazetidin-3-yl)oxy]phenyl}pyridin-2(1H)-one, 1-[4-(azetidin-3-yloxy)phenyl]-4-[(5-chloropyridin-2-yl)methoxy]pyridin-2(1H)-one, 4-[(5-chloropyridin-2-yl)methoxy]-1-{4-[(1-ethylazetidin-3-yl)oxy]phenyl}pyridin-2(1H)-one, 4-[(5-chloropyridin-2-yl)methoxy]-1-{4-[(1-propylazetidin-3-yl)oxy]phenyl}pyridin-2(1H)-one, and 4-[(5-chloropyridin-2-yl)methoxy]-1-(4-{[(2S)-1-ethylazetidin-2-yl]methoxy}phenyl)pyridin-2(1H)-one, or a pharmaceutically acceptable salt thereof.

Specific DP-IV inhibitors of use in combination with a compound of the present invention are selected from 7-[(3R)-3-amino-4-(2,4,5-trifluorophenyl)butanoyl]-3-(trifluoromethyl)-5,6,7,8-tetrahydro-1,2,4-triazolo[4,3-a]pyrazine. In particular, the compound of formula I is favorably combined with 7-[(3R)-3-amino-4-(2,4,5-trifluorophenyl)butanoyl]-3-(trifluoromethyl)-5,6,7,8-tetrahydro-1,2,4-triazolo[4,3-a]pyrazine, and pharmaceutically acceptable salts thereof.

Specific H3 (histamine H3) antagonists/inverse agonists of use in combination with a compound of the present invention include: those described in WO05/077905, including: 3-{4-[(1-cyclobutyl-4-piperidinyl)oxy]phenyl}-2-ethylpyrido[2,3-d]-pyrimidin-4(3H)-one, 3-{4-[(1-cyclobutyl-4-piperidinyl)oxy]phenyl}-2-methylpyrido[4,3-d]pyrimidin-4(3H)-one, 2-ethyl-3-(4-{3-[(3S)-3-methylpiperidin-1-yl]propoxy}phenyl)pyrido[2,3-d]pyrimidin-4(3H)-one 2-methyl-3-(4-{3-[(3S)-3-methylpiperidin-1-yl]propoxy}phenyl)pyrido[4,3-d]pyrimidin-4(3H)-one, 3-{4-[(1-cyclobutyl-4-piperidinyl)oxy]phenyl}-2,5-dimethyl-4(3H)-quinazolinone, 3-{4-[(1-cyclobutyl-4-piperidinyl)oxy]phenyl}-2-methyl-5-trifluoromethyl-4(3H)-quinazolinone, 3-{4-[(1-cyclobutyl-4-piperidinyl)oxy]phenyl}-5-methoxy-2-methyl-4(3H)-quinazolinone, 3-{4-[(1-cyclobutylpiperidin-4-yl)oxy]phenyl}-5-fluoro-2-methyl-4(3H)-quinazolinone, 3-{4-[(1-cyclobutylpiperidin-4-yl)oxy]phenyl}-7-fluoro-2-methyl-4(3H)-quinazolinone, 3-{4-[(1-cyclobutylpiperidin-4-yl)oxy]phenyl}-6-methoxy-2-methyl-4(3H)-quinazolinone, 3-{4-[(1-cyclobutylpiperidin-4-yl)oxy]phenyl}-6-fluoro-2-methyl-4(3H)-quinazolinone, 3-{4-[(1-cyclobutylpiperidin-4-yl)oxy]phenyl}-8-fluoro-2-methyl-4(3H)-quinazolinone, 3-{4-[(1-cyclopentyl-4-piperidinyl)oxy]phenyl}-2-methylpyrido[4,3-d]pyrimidin-4(3H)-one, 3-{4-[(1-cyclobutylpiperidin-4-yl)oxy]phenyl}-6-fluoro-2-methylpyrido[3,4-d]pyrimidin-4(3H)-one, 3-{4-[(1-cyclobutyl-4-piperidinyl)oxy]phenyl}-2-ethylpyrido[4,3-d]pyrimidin-4(3H)-one, 6-methoxy-2-methyl-3-{4-[3-(1-piperidinyl)propoxy]phenyl}pyrido[3,4-d]pyrimidin-4(3H)-one, 6-methoxy-2-methyl-3-{4-[3-(1-pyrrolidinyl)propoxy]phenyl}pyrido[3,4-d]pyrimidin-4(3H)-one, 2,5-dimethyl-3-{4-[3-(1-pyrrolidinyl)propoxy]phenyl}-4(3H)-quinazolinone, 2-methyl-3-{4-[3-(1-pyrrolidinyl)propoxy]phenyl}-5-trifluoromethyl-4(3H)-quinazolinone, 5-fluoro-2-methyl-3-{4-[3-(1-piperidinyl)propoxy]phenyl}-4(3H)-quinazolinone, 6-methoxy-2-methyl-3-{4-[3-(1-piperidinyl)propoxy]phenyl}-4(3H)-quinazolinone, 5-methoxy-2-methyl-3-(4-{3-[(3S)-3-methylpiperidin-1-yl]propoxy}phenyl)-4(3H)-quinazolinone, 7-methoxy-2-methyl-3-(4-{3-[(3S)-3-methylpiperidin-1-yl]propoxy}phenyl)-4(3H)-quinazolinone, 2-methyl-3-(4-{3-[(3S)-3-methylpiperidin-1-yl]propoxy}phenyl)pyrido[2,3-d]pyrimidin-4(3H)-one, 5-fluoro-2-methyl-3-(4-{3-[(2R)-2-methylpyrrolidin-1-yl]propoxy}phenyl)-4(3H)-quinazolinone, 2-methyl-3-(4-{3-[(2R)-2-methylpyrrolidin-1-yl]propoxy}phenyl)pyrido[4,3-d]pyrimidin-4(3H)-one, 6-methoxy-2-methyl-3-(4-{3-[(2R)-2-methylpyrrolidin-1-yl]propoxy}phenyl)-4(3H)-quinazolinone, 6-methoxy-2-methyl-3-(4-{3-[(2S)-2-methylpyrrolidin-1-yl]propoxy}phenyl)-4(3H)-quinazolinone, and pharmaceutically acceptable salts thereof.

Specific CCK1R agonists of use in combination with a compound of the present invention include: 3-(4-{[1-(3-ethoxyphenyl)-2-(4-methylphenyl)-1H-imidazol-4-yl]carbonyl}-1-piperazinyl)-1-naphthoic acid; 3-(4-{[1-(3-ethoxyphenyl)-2-(2-fluoro-4-methylphenyl)-1H-imidazol-4-yl]carbonyl}-1-piperazinyl)-1-naphthoic acid; 3-(4-{[1-(3-ethoxyphenyl)-2-(4-fluorophenyl)-1H-imidazol-4-yl]carbonyl}-1-piperazinyl)-1-naphthoic acid; 3-(4-{[1-(3-ethoxyphenyl)-2-(2,4-difluorophenyl)-1H-imidazol-4-yl]carbonyl}-1-piperazinyl)-1-naphthoic acid; and 3-(4-{[1-(2,3-dihydro-1,4-benzodioxin-6-yl)-2-(4-fluorophenyl)-1H-imidazol-4-yl]carbonyl}-1-piperazinyl)-1-naphthoic acid; and pharmaceutically acceptable salts thereof.

Specific MC4R agonists of use in combination with a compound of the present invention include: 1) (5S)-1′-{[(3R,4R)-1-tert-butyl-3-(2,3,4-trifluorophenyl)piperidin-4-yl]carbonyl}-3-chloro-2-methyl-5-[1-methyl-1-(1-methyl-1H-1,2,4-triazol-5-yl)ethyl]-5H-spiro[furo[3,4-b]pyridine-7,4′-piperidine]; 2) (SR)-1′-{[(3R,4R)-1-tert-butyl-3-(2,3,4-trifluorophenyl)-piperidin-4-yl]carbonyl}-3-chloro-2-methyl-5-[1-methyl-1-(1-methyl-1H-1,2,4-triazol-5-yl)ethyl]-5H-spiro[furo[3,4-b]pyridine-7,4′-piperidine]; 3) 2-(1′-{[(3S,4R)-1-tert-butyl-4-(2,4-difluorophenyl)pyrrolidin-3-yl]carbonyl}-3-chloro-2-methyl-5H-spiro[furo[3,4-b]pyridine-7,4′-piperidin]-5-yl)-2-methylpropanenitrile; 4) 1′-{[(3S,4R)-1-tert-butyl-4-(2,4-difluorophenyl)pyrrolidin-3-yl]carbonyl}-3-chloro-2-methyl-5-[1-methyl-1-(1-methyl-1H-1,2,4-triazol-5-yl)ethyl]-5H-spiro[furo[3,4-b]pyridine-7,4′-piperidine]; 5) N-[(3R,4R)-3-({3-chloro-2-methyl-5-[1-methyl-1-(1-methyl-1H-1,2,4-triazol-5-yl)ethyl]-1′H,5H-spiro[furo-[3,4-b]pyridine-7,4′-piperidin]-1′-yl}carbonyl)-4-(2,4-difluorophenyl)-cyclopentyl]-N-methyltetrahydro-2H-pyran-4-amine; 6) 2-[3-chloro-1′-({(1R,2R)-2-(2,4-difluorophenyl)-4-[methyl(tetrahydro-2H-pyran-4-yl)amino]-cyclopentyl}-carbonyl)-2-methyl-5H-spiro[furo[3,4-b]pyridine-7,4′-piperidin]-5-yl]-2-methyl-propane-nitrile; and pharmaceutically acceptable salts thereof.

“Obesity” is a condition in which there is an excess of body fat. The operational definition of obesity is based on the Body Mass Index (BMI), calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk for obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index (BMI) in Asians. In Asian countries, including Japan, “obesity” refers to a condition whereby a subject with at least one obesity-induced or obesity-related co-morbidity that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asian countries, a “subject at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, non-insulin dependent diabetes mellitus—type 2, impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

“Treatment” (of obesity and obesity-related disorders) refers to the administration of the compounds of the present invention to reduce or maintain the body weight of an obese subject. One outcome of treatment may be reducing the body weight of an obese subject relative to that subject's body weight immediately before the administration of the compounds of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

“Prevention” (of obesity and obesity-related disorders) refers to the administration of the compounds of the present invention to reduce or maintain the body weight of a subject at risk of obesity. One outcome of prevention may be reducing the body weight of a subject at risk of obesity relative to that subject's body weight immediately before the administration of the compounds of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type II diabetes, polycystic ovarian disease, cardiovascular diseases, osteoarthritis, dermatological disorders, hypertension, insulin resistance, hypercholesterolemia, hypertriglyceridemia, and cholelithiasis.

The obesity-related disorders herein are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovarian disease, craniopharyngioma, the Prader-Willi Syndrome, Frohlich's syndrome, GH-deficient subjects, normal variant short stature, Tumer's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, also known as syndrome X, insulin resistance syndrome, sexual and reproductive dysfunction, such as infertility, hypogonadism in males and hirsutism in females, gastrointestinal motility disorders, such as obesity-related gastro-esophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer. The compounds of the present invention are also useful for reducing the risk of secondary outcomes of obesity, such as reducing the risk of left ventricular hypertrophy.

The compounds of formula I are also useful for treating or preventing obesity and obesity-related disorders in cats and dogs. As such, the term “mammal” includes companion animals such as cats and dogs.

The term “diabetes,” as used herein, includes both insulin-dependent diabetes mellitus (IDDM, also known as type I diabetes) and non-insulin-dependent diabetes mellitus (NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compounds of the present invention are useful for treating both Type I and Type II diabetes. The compounds are especially effective for treating Type II diabetes. The compounds of the present invention are also useful for treating and/or preventing gestational diabetes mellitus.

It will be appreciated that for the treatment or prevention of migraine, a compound of the present invention may be used in conjunction with other anti-migraine agents, such as ergotamines or 5-HT₁ agonists, especially sumatriptan, naratriptan, zolmatriptan or rizatriptan.

It will be appreciated that for the treatment of depression or anxiety, a compound of the present invention may be used in conjunction with other anti-depressant or anti-anxiety agents.

Suitable classes of anti-depressant agents include norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists and atypical anti-depressants.

Suitable norepinephrine reuptake inhibitors include tertiary amine tricyclics and secondary amine tricyclics. Suitable examples of tertiary amine tricyclics include: amitriptyline, clomipramine, doxepin, imipramine and trimipramine, and pharmaceutically acceptable salts thereof. Suitable examples of secondary amine tricyclics include: amoxapine, desipramine, maprotiline, nortriptyline and protriptyline, and pharmaceutically acceptable salts thereof.

Suitable selective serotonin reuptake inhibitors include: fluoxetine, fluvoxamine, paroxetine, imipramine and sertraline, and pharmaceutically acceptable salts thereof.

Suitable monoamine oxidase inhibitors include: isocarboxazid, phenelzine, tranylcypromine and selegiline, and pharmaceutically acceptable salts thereof.

Suitable reversible inhibitors of monoamine oxidase include: moclobemide, and pharmaceutically acceptable salts thereof.

Suitable serotonin and noradrenaline reuptake inhibitors of use in the present invention include: venlafaxine, and pharmaceutically acceptable salts thereof.

Suitable CRF antagonists include those compounds described in International Patent Specification Nos. WO 94/13643, 94/13644, 94/13661, 94/13676 and 94/13677. Still further, neurokinin-1 (NK-1) receptor antagonists may be favorably employed with the CB1 receptor modulators of the present invention. NK-1 receptor antagonists of use in the present invention are fully described in the art. Specific neurokinin-1 receptor antagonists of use in the present invention include: (±)-(2R3R,2S3S)—N-{[2-cyclopropoxy-5-(trifluoromethoxy)-phenyl]methyl}-2-phenylpiperidin-3-amine; 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine; aperpitant; CJ17493; GW597599; GW679769; R673; RO67319; R1124; R1204; SSR146977; SSR240600; T-2328; and T2763.; or a pharmaceutically acceptable salts thereof.

Suitable atypical anti-depressants include: bupropion, lithium, nefazodone, trazodone and viloxazine, and pharmaceutically acceptable salts thereof.

Suitable classes of anti-anxiety agents include benzodiazepines and 5-HT_{1A} agonists or antagonists, especially 5-HT_{1A} partial agonists, and corticotropin releasing factor (CRF) antagonists. Suitable benzodiazepines include: alprazolam, chlordiazepoxide, clonazepam, chlorazepate, diazepam, halazepam, lorazepam, oxazepam and prazepam, and pharmaceutically acceptable salts thereof. Suitable 5-HT_{1A} receptor agonists or antagonists include, in particular, the 5-HT_{1A} receptor partial agonists buspirone, flesinoxan, gepirone and ipsapirone, and pharmaceutically acceptable salts thereof. Suitable corticotropin releasing factor (CRF) antagonists include those previously discussed herein.

As used herein, the term “substance abuse disorders” includes substance dependence or abuse with or without physiological dependence. The substances associated with these disorders are: alcohol, amphetamines (or amphetamine-like substances), caffeine, cannabis, cocaine, hallucinogens, inhalants, marijuana, nicotine, opioids, phencyclidine (or phencyclidine-like compounds), sedative-hypnotics or benzodiazepines, and other (or unknown) substances and combinations of all of the above.

In particular, the term “substance abuse disorders” includes drug withdrawal disorders such as alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances. It will be appreciated that reference to treatment of nicotine withdrawal includes the treatment of symptoms associated with smoking cessation.

Other “substance abuse disorders” include substance-induced anxiety disorder with onset during withdrawal; substance-induced mood disorder with onset during withdrawal; and substance-induced sleep disorder with onset during withdrawal.

In particular, compounds of structural formula I are useful for aiding in stopping consumption of tobacco and are useful in treating nicotine dependence and nicotine withdrawal. The compounds of formula I produce in consumers of nicotine, such as tobacco smokers, a total or partial abstinence from smoking. Further, withdrawal symptoms are lessened and the weight gain that generally accompanies quitting tobacco comsumption is reduced or nonexistent. For smoking cessation, the compound of form I may be used in combination with a nicotine agonist or a partial nicotine agonist, including varenicline and selective alpha-4 beta 2 nicotinic partial agonists such as SSR 591813, or a monoamine oxidase inhibitor (MAOI), or another active ingredient demonstrating efficacy in aiding cessation of tobacco consumption; for example, an antidepressant such as bupropion, doxepine, ornortriptyline; or an anxiolytic such as buspirone or clonidine.

It will be appreciated that a combination of a conventional antipsychotic drug with a CB1 receptor modulator may provide an enhanced effect in the treatment of mania. Such a combination would be expected to provide for a rapid onset of action to treat a manic episode thereby enabling prescription on an “as needed basis”. Furthermore, such a combination may enable a lower dose of the antispychotic agent to be used without compromising the efficacy of the antipsychotic agent, thereby minimizing the risk of adverse side-effects. A yet further advantage of such a combination is that, due to the action of the CB1 receptor modulator, adverse side-effects caused by the antipsychotic agent such as acute dystonias, dyskinesias, akathesia and tremor may be reduced or prevented.

Thus, according to a further aspect of the present invention there is provided the use of a CB1 receptor modulator and an antipsychotic agent for the manufacture of a medicament for the treatment or prevention of mania.

The present invention also provides a method for the treatment or prevention of mania, which method comprises administration to a patient in need of such treatment or at risk of developing mania of an amount of a CB1 receptor modulator and an amount of an antipsychotic agent, such that together they give effective relief.

In a further aspect of the present invention, there is provided a pharmaceutical composition comprising a CB1 receptor modulator and an antipsychotic agent, together with at least one pharmaceutically acceptable carrier or excipient, wherein the CB1 receptor modulator and the antipsychotic agent may be present as a combined preparation for simultaneous, separate or sequential use for the treatment or prevention of mania. Such combined preparations may be, for example, in the form of a twin pack.

In a further or alternative aspect of the present invention, there is therefore provided a product comprising a CB1 receptor modulator and an antipsychotic agent as a combined preparation for simultaneous, separate or sequential use in the treatment or prevention of mania.

It will be appreciated that when using a combination of the present invention, the CB1 receptor modulator and the antipsychotic agent may be in the same pharmaceutically acceptable carrier and therefore administered simultaneously. They may be in separate pharmaceutical carriers such as conventional oral dosage forms which are taken simultaneously. The term “combination” also refers to the case where the compounds are provided in separate dosage forms and are administered sequentially. Therefore, by way of example, the antipsychotic agent may be administered as a tablet and then, within a reasonable period of time, the CB1 receptor modulator may be administered either as an oral dosage form such as a tablet or a fast-dissolving oral dosage form. By a “fast-dissolving oral formulation” is meant, an oral delivery form which when placed on the tongue of a patient, dissolves within about 10 seconds.

Included within the scope of the present invention is the use of CB1 receptor modulators in combination with an antipsychotic agent in the treatment or prevention of hypomania.

It will be appreciated that a combination of a conventional antipsychotic drug with a CB1 receptor modulator may provide an enhanced effect in the treatment of schizophrenic disorders. Such a combination would be expected to provide for a rapid onset of action to treat schizophrenic symptoms thereby enabling prescription on an “as needed basis”. Furthermore, such a combination may enable a lower dose of the CNS agent to be used without compromising the efficacy of the antipsychotic agent, thereby minimizing the risk of adverse side-effects. A yet further advantage of such a combination is that, due to the action of the CB1 receptor modulator, adverse side-effects caused by the antipsychotic agent such as acute dystonias, dyskinesias, akathesia and tremor may be reduced or prevented.

As used herein, the term “schizophrenic disorders” includes paranoid, disorganized, catatonic, undifferentiated and residual schizophrenia; schizophreniform disorder; schizoaffective disorder; delusional disorder; brief psychotic disorder; shared psychotic disorder; substance-induced psychotic disorder; and psychotic disorder not otherwise specified.

Other conditions commonly associated with schizophrenic disorders include self-injurious behavior (e.g. Lesch-Nyhan syndrome) and suicidal gestures.

Suitable antipsychotic agents of use in combination with a CB1 receptor modulator include the phenothiazine, thioxanthene, heterocyclic dibenzazepine, butyrophenone, diphenylbutylpiperidine and indolone classes of antipsychotic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. Suitable examples of dibenzazepines include clozapine and olanzapine. An example of a butyrophenone is haloperidol. An example of a diphenylbutylpiperidine is pimozide. An example of an indolone is molindolone. Other antipsychotic agents include loxapine, sulpiride and risperidone. It will be appreciated that the antipsychotic agents when used in combination with a CB1 receptor modulator may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, olanzapine, haloperidol, pimozide and risperidone are commonly used in a non-salt form.

Other classes of antipsychotic agent of use in combination with a CB1 receptor modulator include dopamine receptor antagonists, especially D2, D3 and D4 dopamine receptor antagonists, and muscarinic ml receptor agonists. An example of a D3 dopamine receptor antagonist is the compound PNU-99194A. An example of a D4 dopamine receptor antagonist is PNU-101387. An example of a muscarinic ml receptor agonist is xanomeline.

Another class of antipsychotic agent of use in combination with a CB1 receptor modulator is the 5-HT_{2A} receptor antagonists, examples of which include MDL100907 and fananserin. Also of use in combination with a CB1 receptor modulator are the serotonin dopamine antagonists (SDAs) which are believed to combine 5-HT_{2A} and dopamine receptor antagonist activity, examples of which include olanzapine and ziperasidone.

Still further, NK-1 receptor antagonists may be favorably employed with the CB1 receptor modulators of the present invention. Preferred NK-1 receptor antagonists for use in the present invention are selected from the classes of compounds described previously.

It will be appreciated that a combination of a conventional anti-asthmatic drug with a CB1 receptor modulator may provide an enhanced effect in the treatment of asthma, and may be used for the treatment or prevention of asthma, which method comprises administration to a patient in need of such treatment an amount of a compound of the present invention and an amount of an anti-asthmatic agent, such that together they give effective relief.

Suitable anti-asthmatic agents of use in combination with a compound of the present invention include, but are not limited to: (a) VLA-4 antagonists such as natalizumab and the compounds described in U.S. Pat. No. 5,510,332, WO97/03094, WO97/02289, WO96/40781, WO96/22966, WO96/20216, WO96/01644, WO96/06108, WO95/15973 and WO96/31206; (b) steroids and corticosteroids such as beclomethasone, methylprednisolone, betamethasone, prednisone, dexamethasone, and hydrocortisone; (c) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, desloratadine, cetirizine, fexofenadine, descarboethoxyloratadine, and the like; (d) non-steroidal anti-asthmatics including P2-agonists (such as terbutaline, metaproterenol, fenoterol, isoetharine, albuterol, bitolterol, salmeterol, epinephrine, and pirbuterol), theophylline, cromolyn sodium, atropine, ipratropium bromide, leukotriene antagonists (such as zafirlukast, montelukast, pranlukast, iralukast, pobilukast, and SKB-106,203), and leukotriene biosynthesis inhibitors (such as zileuton and BAY-1005); (e) anti-cholinergic agents including muscarinic antagonists (such as ipratropium bromide and atropine); and (f) antagonists of the chemokine receptors, especially CCR-3; and pharmaceutically acceptable salts thereof.

It will be appreciated that a combination of a conventional anti-constipation drug with a CB1 receptor modulator may provide an enhanced effect in the treatment of constipation or chronic intestinal pseudo-obstruction, and for use for the manufacture of a medicament for the treatment or prevention of constipation or chronic intestinal pseudo-obstruction.

The present invention also provides a method for the treatment or prevention of constipation, which method comprises administration to a patient in need of such treatment an amount of a compound of the present invention and an amount of an anti-constipation agent, such that together they give effective relief.

Suitable anti-constipation agents of use in combination with a compound of the present invention include, but are not limited to, osmotic agents, laxatives and detergent laxatives (or wetting agents), bulking agents, and stimulants; and pharmaceutically acceptable salts thereof. A particularly suitable class of osmotic agents include, but are not limited to sorbitol, lactulose, polyethylene glycol, magnesium, phosphate, and sulfate; and pharmaceutically acceptable salts thereof. A particularly suitable class of laxatives and detergent laxatives, include, but are not limited to, magnesium, and docusate sodium; and pharmaceutically acceptable salts thereof. A particularly suitable class of bulking agents include, but are not limited to, psyllium, methylcellulose, and calcium polycarbophil; and pharmaceutically acceptable salts thereof. A particularly suitable class of stimulants include, but are not limited to, anthroquinones, and phenolphthalein; and pharmaceutically acceptable salts thereof.

It will be appreciated that a combination of a conventional anti-cirrhosis drug with a CB1 receptor modulator may provide an enhanced effect in the treatment or prevention of cirrhosis of the liver, and for use for the manufacture of a medicament for the treatment or prevention of cirrhosis of the liver, as well as non-alcoholic fatty liver disease (NAFLD) and non-alcoholic steatohepatitis (NASH).

The present invention also provides a method for the treatment or prevention of cirrhosis of the liver, which method comprises administration to a patient in need of such treatment an amount of a compound of the present invention and an anti-cirrhosis agent, such that together they give effective relief.

Suitable anti-cirrhosis agents of use in combination with a compound of the present invention include, but are not limited to, corticosteroids, penicillamine, colchicine, interferon-γ, 2-oxoglutarate analogs, prostaglandin analogs, and other anti-inflammatory drugs and antimetabolites such as azathioprine, methotrexate, leflunamide, indomethacin, naproxen, and 6-mercaptopurine; and pharmaceutically acceptable salts thereof.

The method of treatment of this invention comprises a method of modulating the CB1 receptor and treating CB1 receptor mediated diseases by administering to a patient in need of such treatment a non-toxic therapeutically effective amount of a compound of this invention that selectively antagonizes the CB1 receptor in preference to the other CB or G-protein coupled receptors.

The term “therapeutically effective amount” means the amount the compound of structural formula I that will elicit the biological or medical response of a tissue, system, animal or human that is being sought by the researcher, veterinarian, medical doctor or other clinician, which includes alleviation of the symptoms of the disorder being treated. The novel methods of treatment of this invention are for disorders known to those skilled in the art. The term “mammal” includes humans, and companion animals such as dogs and cats.

The weight ratio of the compound of the Formula I to the second active ingredient may be varied and will depend upon the effective dose of each ingredient. Generally, an effective dose of each will be used. Thus, for example, when a compound of the Formula I is combined with a β-3 agonist the weight ratio of the compound of the Formula I to the β-3 agonist will generally range from about 1000:1 to about 1:1000, preferably about 200:1 to about 1:200. Combinations of a compound of the Formula I and other active ingredients will generally also be within the aforementioned range, but in each case, an effective dose of each active ingredient should be used.

In order to illustrate the invention, the following schemes, examples and intermediates are included. These schemes, examples and intermediates do not limit the invention. They are only meant to suggest a method of reducing the invention to practice. Those skilled in the art may find other methods of practicing the invention which are readily apparent to them. However, those methods are also deemed to be within the scope of this invention.

The following reaction schemes illustrate methods which may be employed for the synthesis of the novel pyrido[3,2-e][1,2,4]triazolo[4,3-c]pyrimidines of structural formula I described in this invention. All substituents are as defined above unless indicated otherwise. There are two embodiments of the title compounds of general formula I that are within the scope of this invention as shown in Figure 1. The first embodiment of the title compounds of general formula I are the substituted pyrido[3,2-e][1,2,4]triazolo[4,3-c]pyrimidin-3(2H)-ones shown in general formula II, wherein the Z group is a carbonyl group, the optional double bond between the nitrogen atom at position 2 and the group Z is absent, and the nitrogen at position 2 is bonded to the R² substituent which is defined above. The second embodiment of the title compounds of general formula I are the substituted pyrido[3,2-e][1,2,4]triazolo[4,3-c]pyrimidines shown in general formula III, wherein the Z group is a carbon atom substituted with the substituent R⁸ as defined above, the optional double bond between the nitrogen atom at position 2 is present, the group Z is present, and the R² substituent is absent.

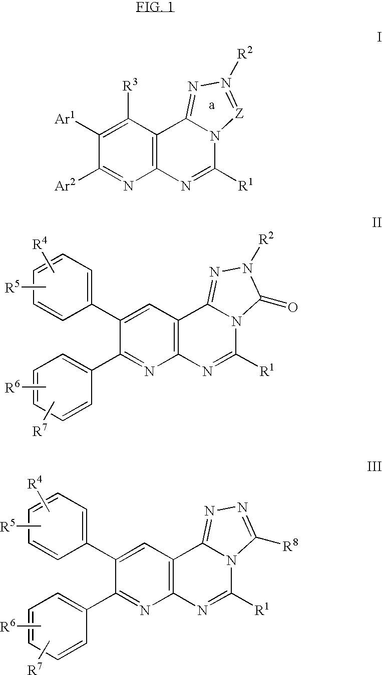

Several strategies based upon synthetic transformations known in the literature of organic synthesis may be employed for the preparation of the title compounds of general formula I. A preferred synthetic process which is shown in the retrosynthetic sense in reaction Scheme 1 begins with a 1,2-diarylethanone of general formula 1 wherein Ar¹ and Ar² are optionally substituted with substituents R⁴, R⁵, R⁶, and R⁷. The 1,2-diarylethanone of general formula 1 is first converted to a 3-cyano-2-pyridone of general formula 2 and then to a substituted pyrido[2,3-d]pyrimidin-4(3H)-one of general formula 3 using the methods described in reaction Scheme 3 as shown below. Finally, the pyrido[2,3-d]pyrimidin-4(3H)-one of general formula 3 may either be converted to the pyrido[3,2-e][1,2,4]triazolo[4,3-c]pyrimidin-3(2H)-one of general formula II as shown in reaction Scheme 4, or the pyrido[2,3-d]pyrimidin-4(3H)-one of general formula 3 may alternatively be converted to the substituted pyrido[3,2-e][1,2,4]-triazolo[4,3-c]pyrimidines of general formula III as shown in reaction Scheme 5.

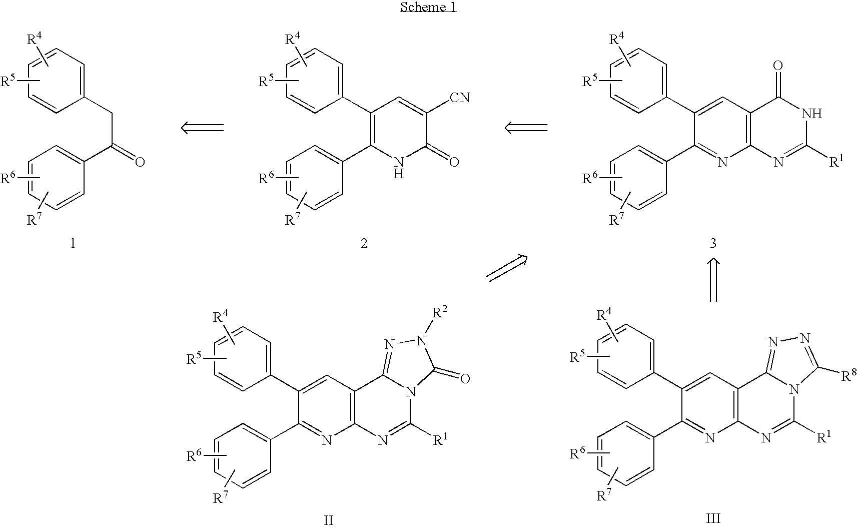

1,2-Diarylethanones of general formula 1 may be available commercially or they can be synthesized using one of several methods known in the art of organic synthesis. Scheme 2 illustrates two methods for the synthesis of the 1,2-diarylethanones of general formula 1. In the first example (equation 1), a substituted arylmethyl bromide of general formula 4 is converted to a Grignard reagent with magnesium metal in a solvent such as THF at a temperature between room temperature and the refluxing temperature of the solvent. The resulting Grignard reagent is then added to a substituted arylnitrile of general formula 5. Acidic hydrolysis of the reaction mixture followed by extraction of the organic product affords a 1,2-diarylethanone of general formula 1 as shown. An alternative synthesis of 1,2-diarylethanones 1 which is preferred when either of the aryl groups is optionally substituted with functional groups that are reactive with Grignard reagents is shown at the bottom of reaction Scheme 2 (equation 2). Here a substituted arylacetic acid of general formula 6 is reacted at low temperature (−78° to −50° C.) with two equivalents of a strong base such as lithium bis(trimethylsilylamide) in an aprotic solvent such as THF. This doubly deprotonates the arylacetic acid 6 and generates a dianion which undergoes a Dieckmann reaction when the substituted arylcarboxylate ester of general formula 7 is added. In this modification of the Dieckmann reaction, the intermediate β-keto acid smoothly decarboxylates and a 1,2-diarylethanone of general formula 1 is produced.

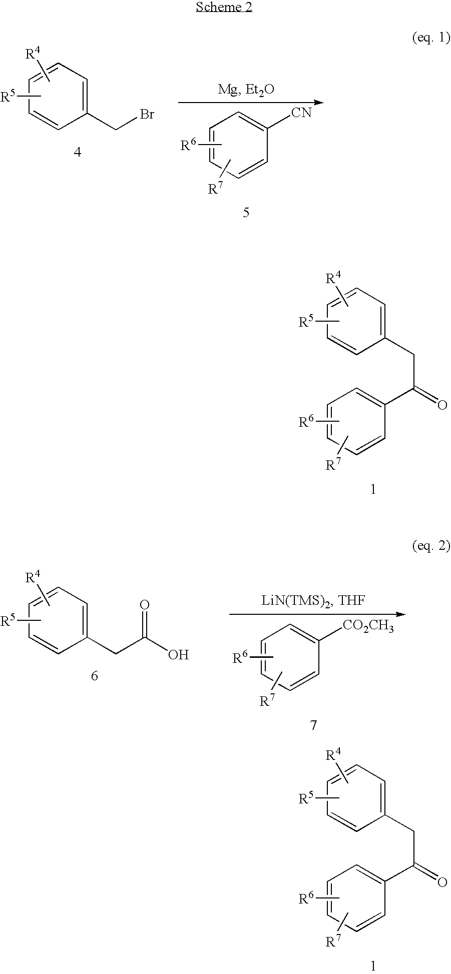

Reaction Scheme 3 illustrates the method for the conversion of the 1,2-diarylethanone of general formula 1 into the 3-cyano-2-pyridones of general formula 2 and then to the pyrido[2,3-d]pyrimidin-4(3H)-ones of general formula 3. The 1,2-diarylethanone of general formula 1 is first converted to a vinylogous amide of general formula 8 by reaction with an N,N-dimethylformamide dimethylacetal as shown. The condensation reaction is conducted using the DMF acetal as the reaction solvent at an elevated temperature, typically between room temperature and 150° C., and the vinylogous amide 8 is produced as a mixture of E and Z diastereoisomers. In the next step of this sequence, the vinylogous amide 8 is condensed with cyanoacetamide to afford the 3-cyano-2-pyridone of general formula 2. The reaction is usually conducted in a polar aprotic solvent such as DMF in the presence of a strong base such as an alkali metal hydride or alkoxide. The 3-cyano-2-pyridone of general formula 2 is then converted to the 2-chloro-3-cyanopyridine derivative of general formula 9 using a chlorinating agent such as phosphorus oxychloride. The reaction is usually conducted at an elevated temperature, for instance between 80° C. and 120° C., and using several equivalents of the phosphorus oxychloride in an inert solvent such as toluene, xylene or the like. Alternatively the reaction may be conducted in neat phosphorus oxychloride. The resulting 2-chloro-3-cyanopyridine derivative of general formula 9 is then converted to a substituted pyrido[2,3-d]pyrimidin-4(3H)-one of general formula 3 by reaction with a substituted amidine of general formula 10. This reaction is typically conducted in a polar aprotic solvent such as dimethylacetamide, N-methylpyrrolidinone or the like, at an elevated temperature, for instance between 100 to 150° C. and in the presence of a base such as DBU. Amidines of general formula 10 are frequently obtained as salts such as hydrochloride salts, and in those cases an excess of the base (e.g. DBU) is employed in the reaction. As the reaction proceeds to completion, it is generally necessary to hydrolyze the imine that results from addition of one of the amino groups from the amidine of general formula 10 to the cyano substituent of the 3-cyanopyridine derivative 9. In some cases the hydrolysis proceeds readily upon addition of water, but in some instances it is preferable to add a strong aqueous acid, such as methanesulfonic acid, at the end of the reaction to ensure complete hydrolysis.

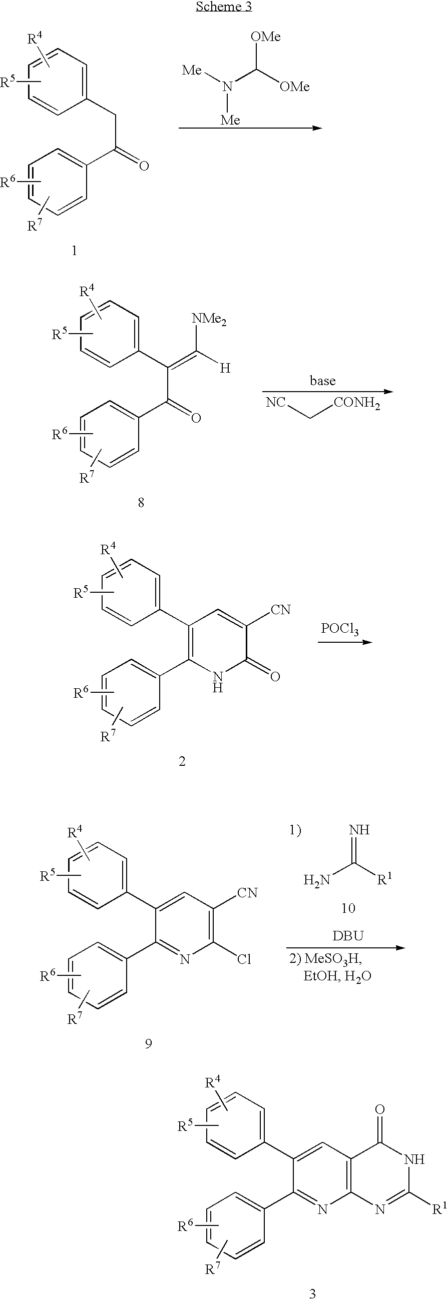

Reaction Scheme 4 illustrates the final stage in the synthesis of the title compounds of general formula I when it is desired to prepare compounds corresponding to embodiment II of the present invention. In this scheme, the substituted pyrido[2,3-d]pyrimidin-4(3H)-one of general formula 3 is first converted to the 4-chloropyrido[2,3-d]pyrimidine derivative of general formula 11 by reaction with a suitable chlorinating reagent such as phosphorus oxychloride. This reaction is also usually conducted at an elevated temperature, for instance between 80° C. and 120° C., using several equivalents of the phosphorus oxychloride in an inert solvent such as toluene, xylene or the like. Alternatively the reaction may also be conducted in neat phosphorus oxychloride. The resulting 4-chloropyrido[2,3-d]pyrimidine derivative of general formula 11 is then reacted with a hydrazine derivative of general formula 12 to afford the 4-hydrazino-pyrido[2,3-d]pyrimidine of general formula 13. This reaction is typically conducted in an inert solvent such as THF, and the reaction generally proceeds in a temperature range between room temperature and the reflux temperature of the solvent. When the substituted hydrazine of general formula 12 is obtained as a salt, such as a hydrochloride salt, then a base such as triethylamine, diisopropylethylamine or DBU is added to accelerate the reaction. The resulting 4-hydrazino-pyrido[2,3-d]pyrimidine of general formula 13 is finally reacted with a phosgene equivalent to afford the compounds of general formula II. The phosgene equivalent may be phosgene itself, or a more conveniently handled reagent such as N,N′-carbonyldiimidazole, trichloromethyl chloroformate or the like. The reaction is typically conducted at temperatures between room temperature and the reflux temperature of the solvent, in an aprotic solvent such dichloromethane, THF, DMF or the like, and the solvent is selected depending upon the solubility characteristics of the starting material 13.

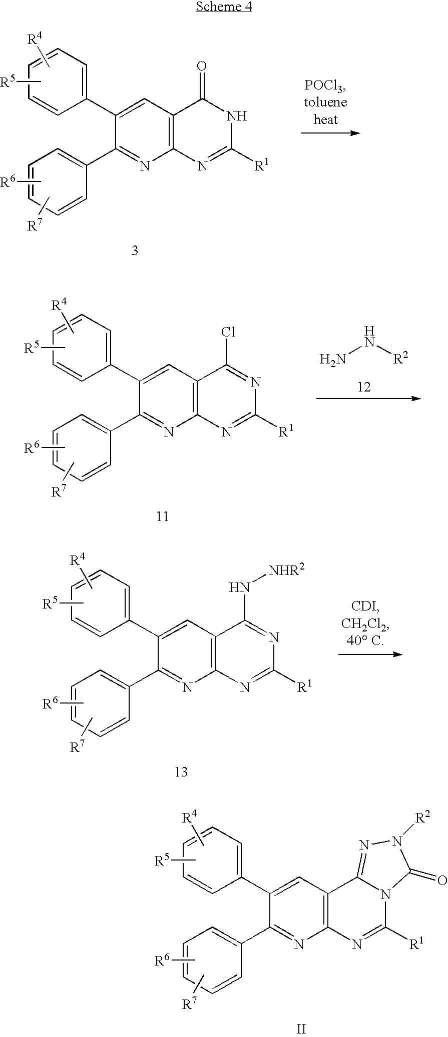

Reaction Scheme 5 illustrates the final stage in the synthesis of the title compounds of general formula I when it is desired to prepare compounds corresponding to embodiment III of the present invention. In this scheme, the 4-chloropyrido[2,3-d]pyrimidine derivative of general formula 11 from Scheme 4, is reacted with hydrazine hydrate to afford the 4-hydrazinopyrido[2,3-d]pyrimidine of general formula 14. This reaction is also typically conducted in an inert solvent like THF, and the reaction generally proceeds in a temperature range between room temperature and the reflux temperature of the solvent. The resulting 4-hydrazinopyrido[2,3-d]pyrimidine of general formula 14 is finally reacted with an orthoester or orthoester equivalent to afford the compounds of general formula III. This latter reaction is typically conducted in a polar aprotic solvent such as dimethylacetamide or N-methyl-pyrrolidinone at an elevated temperature, such as the reflux temperature of the solvent. For instance, when the orthoester (15) selected is triethyl orthoformate (R⁸═H), the reaction is typically conducted in NMP at 135° C. in approximately 18 hours.

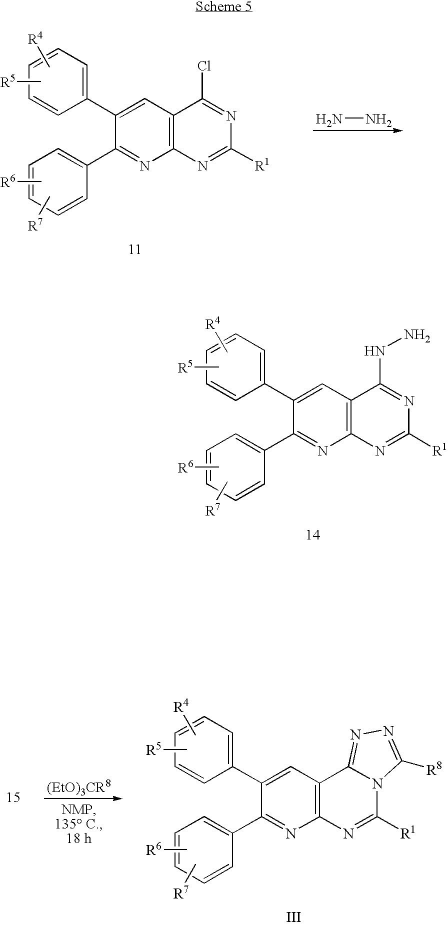

It is to be recognized that the compounds of general formulae II and III described above may be subjected to further synthetic modification to afford additional derivatives which are within the scope of the present invention. For instance, halo substituents on the aromatic rings at the 8- and 9-positions of the compounds of general formulae II and III, may be employed in palladium-catalyzed cross coupling reactions. Numerous palladium catalyzed cross coupling reactions are well known in organic synthesis and are routinely employed to replace halo substituents with a variety of carbon bonded substituent groups including alkyl, vinyl, aryl, cyano and the like. Palladium catalyzed cross coupling reactions that are also well known in the literature of organic chemistry can replace halo substituents with non carbon atom substituents. For instance, palladium-catalyzed cross coupling reactions described by Buchwald can be employed to introduce substituted amino or substituted thio groups. Furthermore, the palladium-catalyzed cross coupling reactions of halo aromatic compounds can be used to prepare organoboron compounds which can be utilized in further cross coupling reactions or the organoboron derivatives may be oxidized under mild conditions to afford phenols.

### General Procedures.

Reactions sensitive to moisture or air were performed under nitrogen or argon using anhydrous solvents and reagents. The progress of reactions was determined by either analytical thin layer chromatography (TLC) performed with E. Merck precoated TLC plates, silica gel 60F-254, layer thickness 0.25 mm or liquid chromatography-mass spectrum (LC-MS). Mass analysis was performed on a Waters Micromass® ZQ™ with electrospray ionization in positive ion detection mode. High performance liquid chromatography (HPLC) was conducted on an Agilent 1100 series HPLC on Waters C18 XTerra 3.5 μm 3.0×50 mm column with gradient 10:90-100 v/v CH₃CN/H₂O+v 0.05% TFA over 3.75 min then hold at 100 CH₃CN+v 0.05% TFA for 1.75 min; flow rate 1.0 mL/min, UV wavelength 254 nm). Concentration of solutions was carried out on a rotary evaporator under reduced pressure. Flash chromatography was performed using a Biotage Flash Chromatography apparatus (Dyax Corp.) on silica gel (32-63 mM, 60 Å pore size) in pre-packed cartridges.

Abbreviations: acetic acid (AcOH), aqueous (aq), (benzotriazol-1-yloxy)tri-pyrrolidinophosphonium hexafluorophosphate (PyBOP), 1,1′-bis(diphenylphosphino)ferrocene (dppf), ethyl (Et), ethyl acetate (EtOAc), diethyl ether (ether or Et₂O), N,N-diisopropylethylamine (DIEA), N,N-dimethylformamide (DMF), 1-methyl-2-pyrrolidinone (NMP), 1,8-diazabicyclo[5.4.0]undec-7-ene (DBU), 4-N,N-dimethylaminopyridine (DMAP), ethylene glycol dimethyl ether (DME), N-bromosuccinimide (NBS), azobisisobutyronitrile (AIBN), dimethyl sulfoxide (DMSO), 1-hydroxybenzotriazole (HOBT or HOBt), N-methylmorpholine-N-oxide (NMO), N-methyl-pyrrolidinone (NMP), methanol (MeOH), gram(s) (g), hour(s) (h or hr), microliter(s) (μL), milligram(s) (mg), milliliter(s) (mL), millimole (mmol), mass spectrum (ms or MS), 2-propanol (IPA), retention time (Rₜ), room temperature (rt), saturated aq sodium chloride solution (brine), trifluoroacetic acid (TFA), tetrahydrofuran (THF), and minute(s) (min).

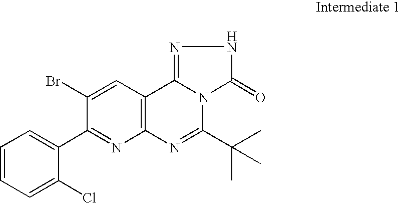

### 9-Bromo-5-tert-butyl-8-(2-chlorophenyl)pyrido[3,2-e][1,2,4]triazolo[4,3-c]pyrimidin-3(2H)-one

Step A: 1-(2-Chlorophenyl)-3-(dimethylamino)prop-2-en-1-one. A solution of 2-chloroacetophenone (188.6 g, 1.22 mol), dimethylformamide dimethylacetal (0.650 L, 4.468 mol) and DMF (3.8 L), was heated at 90° C. for 16 h. The reaction was cooled and water and EtOAc were added and the aqueous layer was extracted twice with EtOAc. The combined organic layer was washed with water and brine and the aqueous layer was re-extracted with EtOAc. The combined organic solutions were dried (Na₂SO₄), filtered and concentrated affording the product.

Step B: 6-(2-Chlorophenyl)-2-oxo-1,2-dihydropyridine-3-carbonitrile. 2-Cyanoacetamide (101 g, 1.19 mol) in DMF (350 mL) was added dropwise to a solution of sodium methoxide (25 wt % in MeOH, 0.55 L, 2.376 mol), in DMF (1.25 L), followed by the slow addition of the product from Step A (227.1 g, 1.08 mol) in DMF (1.5 L). The reaction was heated to 95° C. for 3 h and was allowed to come to rt overnight. The reaction was diluted with water, followed by HCl (2 N), and was stirred for 30 min. Additional water was added and the mixture was filtered and the resulting yellow solid was washed with water, cold ethanol and hexane and placed in a high vacuum oven for 1 day affording the product.

Step C: 5-Bromo-6-(2-chlorophenyl)-2-oxo-1,2-dihydropyridine-3-carbonitrile. The product from Step B (90.88 g, 0.395 mol) was dissolved in MeOH (1.5 L) and 1,4-dioxane (0.75 L) and NBS (70.33 g, 0.395 mol) was added. The reaction was stirred 15 min, diluted with EtOAc, and washed three times with saturated aq NaHCO₃. The aqueous layers were combined and re-extracted twice with EtOAc. The combined organic layers were concentrated and dissolved in isopropanol. Water was added and the solution was concentrated to the point where the product started to come out of solution. The solution was kept in a cold room for 2 days, filtered, and solids obtained were dried in a vacuum oven overnight. The filtrate (mostly aqueous) was saturated with solid NaCl and extracted with EtOAc three times. The combined organic layers were concentrated and re-dissolved in MeOH and water was added. The solution was seeded and allowed to stir overnight. The solution was filtered and the two batches of solids were combined affording the product.

Step D: 5-Bromo-2-chloro-6-(2-chlorophenyl)-1,2-dihydropyridine-3-carbonitrile. The product from Step C (12 g, 38.8 mmol) was dissolved in toluene (50 mL) and phosphorus oxychloride (18 mL, 194 mmol) was added. The mixture was heated at 90° C. for 30 h, followed by 2 days of stirring at rt. The reaction was concentrated, EtOAc was added, and the solution was washed with saturated aq NaHCO₃, dried (Na₂SO₄), filtered and concentrated. The residue was purified by flash chromatography on silica gel gradient eluted with 0-15% EtOAc in hexane affording the product.

Step E: 6-Bromo-2-tert-butyl-7-(2-chlorophenyl)-8,8a-dihydropyrido[2,3-d]pyrimidin-4-amine. To an 80 mL microwave tube of a CEM Corporation Discover 300 Watt microwave reactor, was added the product from Step D (3 g, 9.2 mmol), DMF (10 mL), 2,2-dimethylpropanimidamide hydrochloride (1.87 g, 13.72 mmol) and DBU (2.23 g, 14.6 mmol). The tube was purged with nitrogen, capped and inserted into the microwave reactor and heated at 135° C., 50 watts maximum power, for 25 min. The reaction mixture was concentrated and re-dissolved in EtOAc and hexane and water was added until the desired material precipitated. The solids were isolated and the filtrate was concentrated and was purified by flash chromatography on silica gel gradient eluted with 0-40% EtOAc in hexane. The solids obtained earlier, and the purified material from the silica column, were combined affording the product.

Step F: 6-Bromo-2-tert-butyl-7-(2-chlorophenyl)-8,8a-dihydropyrido[2,3-d]pyrimidin-4(3H)-one. To the product from Step E (2.05 g, 5.23 mmol) was added methanesulfonic acid (9 mL) and the slurry was heated at 110° C. Water (6 mL) was added via syringe once the reaction had reached the desired temperature, and the mixture was heated for 1 h. The solution was cooled and EtOAc and water was added. The organic layer was collected and washed with brine (2 times), saturated aq NaHCO₃ (2 times), water, dried (Na₂SO₄), filtered and concentrated affording the product.

Step G. 6-Bromo-2-tert-butyl-4-chloro-7-(2-chlorophenyl)-8,8a-dihydropyrido[2,3-d]pyrimidine. The product of Step F (1.9 g, 4.84 mmol) was dissolved in toluene (15 mL) and POCl₃ (2.25 mL, 24.2 mmol) was added. The reaction was heated at 110° C. for 1 h and then cooled. EtOAc and water was added and the organic layer was isolated and washed with brine (2 times), saturated aq NaHCO₃ (2 times), water, dried (Na₂SO₄), filtered and concentrated affording the product.

Step H: 6-Bromo-2-tert-butyl-7-(2-chlorophenyl)-4-hydrazino-8,8a-dihydropyrido[2,3-d]pyrimidine. To the product of Step G (0.95 g, 2.31 mmol) in THF (20 mL) was added hydrazine (2.81 mL, 57.8 mmol) at rt. After 20 min, HPLC/MS indicated that all the starting material had been consumed and the reaction was concentrated. The residue was dissolved in EtOAc, washed with brine (5 times), dried (Na₂SO₄), filtered and concentrated affording the product.

Step I: 9-Bromo-5-tert-butyl-8-(2-chlorophenyl)pyrido[3,2-e][1,2,4]-triazolo[4,3-c]pyrimidin-3(2H)-one. To the product of Step H (2.31 mmol), in CH₂Cl₂ (30 mL), was added N,N′-carbonyldiimidazole (1.68 g, 10.4 mmol) and the reaction was allowed to stir at rt overnight. The reaction mixture was concentrated and re-dissolved in EtOAc and washed with brine (3 times), dried (Na₂SO₄), filtered and concentrated. The crude material was taken up in CH₂Cl₂, the solids were isolated and the filtrate was concentrated. The concentrated residue was purified by flash chromatography on silica gel gradient eluted with 0-40% EtOAc in hexane. The solids obtained earlier, and the purified material from the silica column, were combined affording the title compound. HPLC/MS: 432.0 (M+1), 434.0 (M+3); Rₜ=3.48 min.

### Example 1

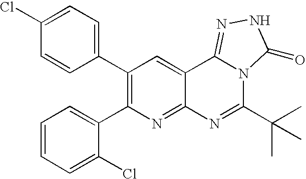

### 5-Tert-butyl-8-(2-chlorophenyl)-9-(4-chlorophenyl)pyrido[3,2-e][1,2,4]triazolo[4,3-c]pyrimidin-3(2H)-one

Step A: 1-(2-Chlorophenyl)-2-(4-chlorophenyl)-3-(dimethylamino)prop-2-en-1-one. To 1-(2-chlorophenyl)-2-(4-chlorophenyl)ethanone (13.2 g, 49.8 mmol) in 100 mL of DMF was added N,N-dimethylformamide dimethyl acetal (23.8 g). The mixture was stirred at 75° C. for 16 h. The solution was concentrated and used without further purification in the next step.

Step B: 6-(2-Chlorophenyl)-5-(4-chlorophenyl)-2-oxo-1,2-dihydropyridine-3-carbonitrile. A solution of all the product from step A dissolved in DMF (80 mL), methanol (4.4 mL) and containing cyanoacetamide (4.61 g, 54.8 mmol) was transferred by cannula into a flask containing a suspension of NaH (4.98 g, 124.5 mmol, 60% dispersion in mineral oil, freed of excess oil by washing with hexane just prior to use) in DMF (40 mL). The solution was heated to 95° C. for 2.5 h then concentrated. The residue was dissolved in ethyl acetate, washed with 10% aq NaHSO₄, water and concentrated to a solid. The solid was suspended in warm ethanol and then cooled, and the title compound was subsequently isolated by filtration and dried in vacuo.

Step C: 2-Chloro-6-(2-chlorophenyl)-5-(4-chlorophenyl)pyridine-3-carbonitrile. To the product of Step B (1.5 g, 4.40 mmol) was added POCl₃ (5 mL). The reaction was heated to 100° C. for 17 h. After cooling to room temperature the excess POCl₃ was removed in vacuo before the residue was dissolved in EtOAc and washed with saturated aq NaHCO₃ solution. The solution was concentrated and purified via flash chromatography on silica gel eluted with 10% EtOAc in hexane affording the product. HPLC/MS: 358.9 (M+1), 360.9 (M+3); Rₜ=4.07 min.

Step D: 2-Tert-butyl-7-(2-chlorophenyl)-6-(4-chlorophenyl)pyrido[2,3-d]pyrimidin-4-amine. An 80 mL CEM corporation Discover microwave tube was charged with the product of Step C (3.1 g, 8.62 mmol), 2,2-dimethylpropanimidamide hydrochloride (1.766 g, 12.93 mmol), DMF (15 mL) and DBU (2.21 mL, 14.7 mmol). The tube was sealed and heated (with air cooling) to 130° C. for 25 min. The reaction was cooled and diluted with EtOAc (250 mL) and MeOH (8 mL). The solution was washed with brine and concentrated. The residue was purified by flash chromatography on silica gel gradient eluted with 0-15% EtOAc in CH₂Cl₂ affording the product. HPLC/MS: 423.1 (M+1), 425.1 (M+3); Rₜ=3.06 min.

Step E: 2-Tert-butyl-7-(2-chlorophenyl)-6-(4-chlorophenyl)pyrido[2,3-d]pyrimidin-4(3H)-one. To the product of Step D (2.05 g, 4.84 mmol) was added methanesulfonic acid (9 mL). The reaction was heated to 113° C. and then water (6 mL) was added. The reaction was stirred for an hour and cooled. It was then diluted with EtOAc, washed with brine, saturated aq NaHCO₃, and brine. The solution was dried (Na₂SO₄), filtered and concentrated to afford the product. HPLC/MS: 424.1 (M+1), 426.1 (M+3); Rₜ=4.04 min.

Step F: 2-Tert-butyl-4-chloro-7-(2-chlorophenyl)-6-(4-chlorophenyl)pyrido[2,3-d]pyrimidine. To the product of Step E (1.90 g, 4.48 mmol) was added POCl₃ (3.43 g, 22.4 mmol) and toluene (15 mL). The reaction was heated to 108° C. for about an hour. The reaction was diluted with EtOAc, washed with brine, saturated aq NaHCO₃, brine and concentrated. The residue was purified via flash chromatography on silica gel gradient eluted with 0-25% EtOAc in hexane affording the product. HPLC/MS: 442.1 (M+1), 444.0 (M+3); Rₜ=4.67 min.

Step G: 2-Tert-butyl-7-(2-chlorophenyl)-6-(4-chlorophenyl)-4-hydrazinopyrido[2,3-d]pyrimidine.

To the product of Step F (252 mg, 0.569 mmol) in THF (7 mL) was added hydrazine hydrate (0.7 mL, 14.4 mmol). The reaction stirred at rt for about 25 min and was concentrated. The residue was diluted with EtOAc, washed with brine (5 times), dried (Na₂SO₄), filtered and concentrated to afford the product which was used without further purification. HPLC/MS: 438.2 (M+1), 440.2 (M+3).

Step H: 5-Tert-butyl-8-(2-chlorophenyl)-9-(4-chlorophenyl)pyrido[3,2-e][1,2,4]triazolo[4,3-c]pyrimidin-3(2H)-one. To the product of Step G (assumed 0.569 mmol) in CH₂Cl₂ (10 mL) was added 1,1′-carbonyldiimidazole (420 mg, 2.59 mmol) and the reaction stirred about 18 h at rt. The reaction was diluted with EtOAc, washed with brine and concentrated. The residue was purified via flash chromatography on silica gel gradient eluted with 0-10% EtOAc in CH₂Cl₂ affording the product.

HPLC/MS: 464.0 (M+1), 466.0 (M+3); Rₜ=3.75 min.

### Example 2

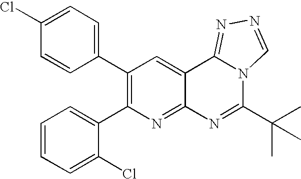

### 5-Tert-butyl-8-(2-chlorophenyl)-9-(4-chlorophenyl)pyrido[3,2-e][1,2,4]-triazolo[4,3-c]pyrimidine

To the product of Example 1 Step G (60.0 mg, 0.137 mmol) was added triethylorthoformate (0.6 mL) and NMP (0.6 mL). The reaction was heated to 135° C. for about 18 h and was concentrated. The residue was diluted with EtOAc, washed with brine and was concentrated. The residue was purified by flash chromatography on silica gel gradient eluted with 0-100% EtOAc in hexane affording the title compound. HPLC/MS: 448.2 (M+1), 450.1 (M+3); Rₜ=3.62 min.

Using the procedures similar to that described in Example 1, and the appropriate starting materials, the following compounds were obtained:

| | | HPLC/MS | |
|---|---|---|---|
| | | m/z (M + 1) | |
| | | m/z (M + 3) | |
| Example | Name | Rₜ (min) | Structure |
| Example 3 | 8-(4-Bromo-2-chlorophenyl)-5-tert- butyl-9-(4-chlorophenyl)pyrido[3,2- e][1,2,4]triazolo[4,3-c]pyrimidin-3(2H)- one | 559.9 561.9 4.03 | 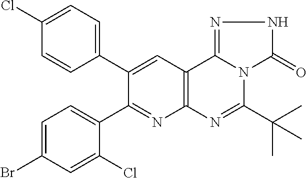 |
| Example 4 | 5-Tert-butyl-9-(4-chlorophenyl)-8-(2- methylphenyl)pyrido[3,2- e][1,2,4]triazolo[4,3-c]pyrimidin-3(2H)- one | 444.1 446.1 3.77 | 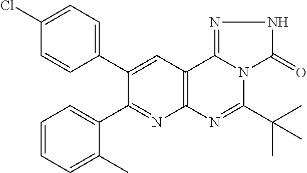 |
| Example 5 | 9-(4-Bromophenyl)-5-tert-butyl-8-(2- chlorophenyl)pyrido[3,2- e][1,2,4]triazolo[4,3-c]pyrimidin-3(2H)- one | 507.8 509.9 3.73 | 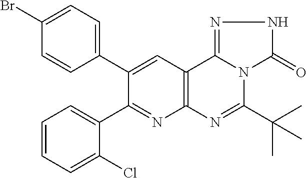 |
| Example 6 | 8-(2-Bromophenyl)-5-tert-butyl-9-(4- chlorophenyl)pyrido[3,2- e][1,2,4]triazolo[4,3-c]pyrimidin-3(2H)- one | 508.1 510.1 3.77 | 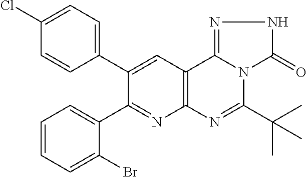 |
| Example 7 | 8-(2-Bromo-4-chlorophenyl)-5-tert- butyl-9-(4-chlorophenyl)pyrido[3,2- e][1,2,4]triazolo[4,3-c]pyrimidin-3(2H)- one | 542.0 544.0 3.97 | 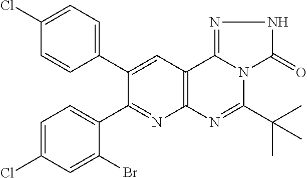 |
| Example 8 | 5-Tert-butyl-8-(2-chloro-3- methylphenyl)-9-(4- chlorophenyl)pyrido[3,2- e][1,2,4]triazolo[4,3-c]pyrimidin-3(2H)- one | 478.1 480.1 3.85 | 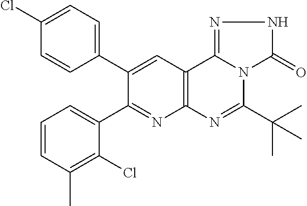 |

### Example 9

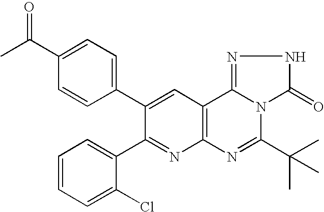

9-(4-Acetylphenyl)-5-tert-butyl-8-(2-chlorophenyl)pyrido[3,2-e][1,2,4]triazolo[4,3-c]pyrimidin-3(2H)-one. The product of Intermediate 1 (0.100 g, 0.231 mmol) was dissolved in DME (0.9 mL), water (0.4 mL) and ethanol (0.2 mL) in a 10 mL reaction tube of a CEM Corporation Discover 300 Watt microwave reactor. An aq solution of Na₂CO₃ (1 M, 0.11 mL, 0.220 mmol), 4-acetylphenylboronic acid (46 mg, 0.277 mmol) and tetrakis(triphenylphosphine)palladium(0) (26.7 mg, 0.023 mmol) were added and the tube was purged with nitrogen, capped and inserted into the microwave reactor and heated at 120° C., 50 watts maximum power, for 3 min. The reaction was diluted with EtOAc, washed with brine, dried (Na₂SO₄), filtered and concentrated. The residue was purified by flash chromatography on silica gel gradient eluted with 0-30% EtOAc in hexane affording the title compound. HPLC/MS: 472.2 (M+1), 474.2 (M+3); Rₜ=3.43 min.

Using the general Suzuki coupling procedure described in Example 9 and the product of Intermediate 1, along with the appropriate boronic acid or boronic ester, the following compounds were obtained:

| | | HPLC/MS | |
|---|---|---|---|
| | | m/z (M + 1) | |
| | | m/z (M + 3) | |
| Example | Name | Rₜ (min) | Structure |
| Example 10 | 5-Tert-butyl-8-(2-chlorophenyl)-9-(3- chlorophenyl)pyrido[3,2- e][1,2,4]triazolo[4,3-c]pyrimidin-3(2H)- one | 464.0 466.0 3.74 | 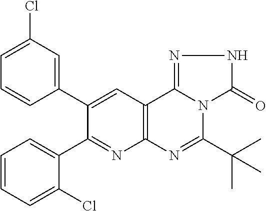 |
| Example 11 | 5-Tert-butyl-8-(2-chlorophenyl)-9-(4- methylphenyl)pyrido[3,2- e][1,2,4]triazolo[4,3-c]pyrimidin-3(2H)- one | 444.1 446.1 3.71 | 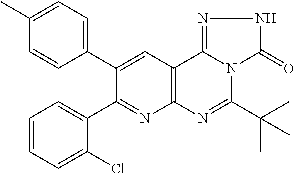 |
| Example 12 | 5-Tert-butyl-8-(2-chlorophenyl)-9-[4- (dimethylamino)phenyl]pyrido[3,2- e][1,2,4]triazolo[4,3-c]pyrimidin-3(2H)- one | 473.1 475.1 3.16 | 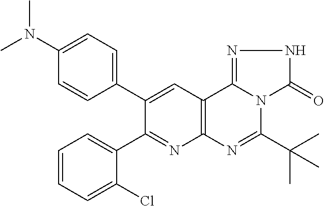 |
| Example 13 | 5-Tert-butyl-8-(2-chlorophenyl)-9-[4- (trifluoromethoxy)phenyl]pyrido[3,2- e][1,2,4]triazolo[4,3-c]pyrimidin-3(2H)- one | 514.0 516.0 3.85 | 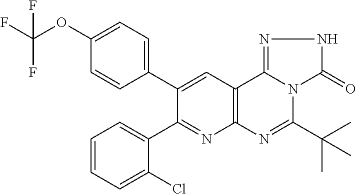 |
| Example 14 | 5-Tert-butyl-8-(2-chlorophenyl)-9-(4- methoxyphenyl)pyrido[3,2- e][1,2,4]triazolo[4,3-c]pyrimidin-3(2H)- one | 460.1 462.1 3.59 | 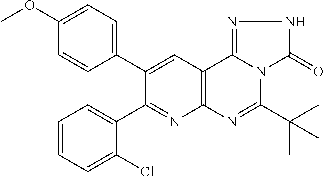 |
| Example 15 | 5-Tert-butyl-8-(2-chlorophenyl)-9-(4- isopropoxyphenyl)pyrido[3,2- e][1,2,4]triazolo[4,3-c]pyrimidin-3(2H)- one | 488.1 490.1 3.81 | 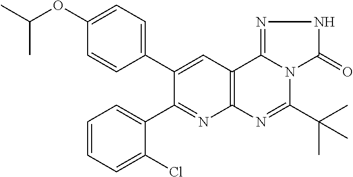 |
| Example 16 | 4-[5-Tert-butyl-8-(2-chlorophenyl)-3- oxo-2,3-dihydropyrido[3,2- e][1,2,4]triazolo[4,3-c]pyrimidin-9- yl]benzonitrile | 455.1 457.0 3.49 | 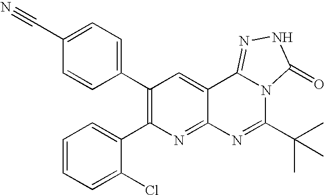 |
| Example 17 | 5-Tert-butyl-8-(2-chlorophenyl)-9-[4- (trifluoromethyl)phenyl]pyrido[3,2- e][1,2,4]triazolo[4,3-c]pyrimidin-3(2H)- one | 498.1 500.0 3.80 | 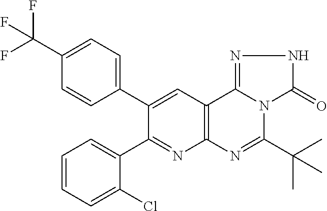 |
| Example 18 | 5-Tert-butyl-8-(2-chlorophenyl)-9-(4- fluorophenyl)pyrido[3,2- e][1,2,4]triazolo[4,3-c]pyrimidin-3(2H)- one | 448.2 450.1 3.60 | 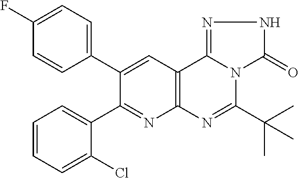 |
| Example 19 | 5-Tert-butyl-8-(2-chlorophenyl)-9-(3,4- difluorophenyl)pyrido[3,2- e][1,2,4]triazolo[4,3-c]pyrimidin-3(2H)- one | 466.2 468.1 3.66 | 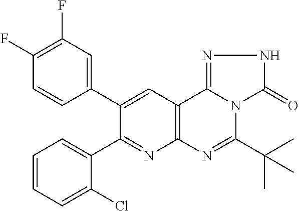 |
| Example 20 | 5-Tert-butyl-8-(2-chlorophenyl)-9-(3,5- difluorophenyl)pyrido[3,2- e][1,2,4]triazolo[4,3-c]pyrimidin-3(2H)- one | 466.2 468.1 3.66 | 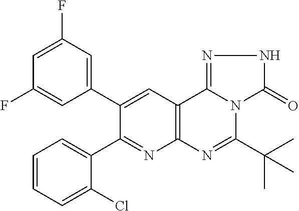 |
| Example 21 | 5-Tert-butyl-9-(4-chloro-3- methylphenyl)-8-(2- chlorophenyl)pyrido[3,2- e][1,2,4]triazolo[4,3-c]pyrimidin-3(2H)- one | 478.1 480.1 3.87 | 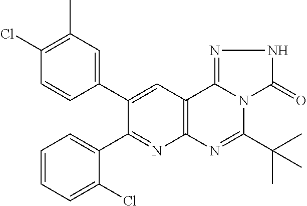 |
| Example 22 | 5-Tert-butyl-8-(2-chlorophenyl)-9-[4- (methylsulfonyl)phenyl]pyrido[3,2- e][1,2,4]triazolo[4,3-c]pyrimidin-3(2H)- one | 508.1 510.1 3.22 | 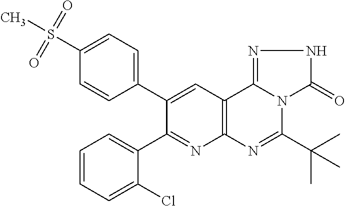 |
| Example 23 | 5-Tert-butyl-8-(2-chlorophenyl)-9- pyridin-4-ylpyrido[3,2- e][1,2,4]triazolo[4,3-c]pyrimidin-3(2H)- one | 431.2 433.2 2.60 | 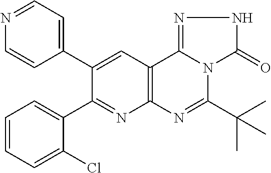 |
| Example 24 | 5-Tert-butyl-8-(2-chlorophenyl)-9- pyridin-3-ylpyrido[3,2- e][1,2,4]triazolo[4,3-c]pyrimidin-3(2H)- one | 431.2 433.2 2.66 | 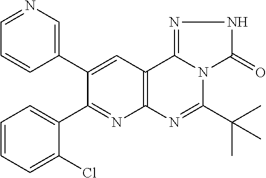 |
| Example 25 | 5-Tert-butyl-8-(2-chlorophenyl)-9-(6- fluoropyridin-3-yl)pyrido[3,2- e][1,2,4]triazolo[4,3-c]pyrimidin-3(2H)- one | 449.2 451.2 3.34 | 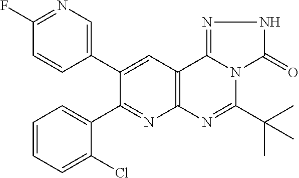 |
| Example 26 | 5-[5-Tert-butyl-8-(2-chlorophenyl)-3- oxo-2,3-dihydropyrido[3,2- e][1,2,4]triazolo[4,3-c]pyrimidin-9- yl]pyridine-2-carbonitrile | 456.2 458.2 3.32 | 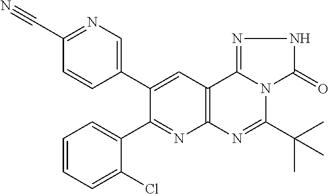 |

### Example 27

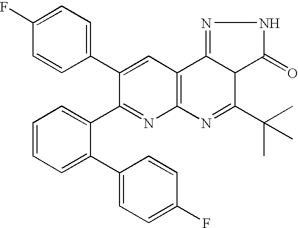

5-Tert-butyl-8-(4′-fluorobiphenyl-2-yl)-9-(4-fluorophenyl)pyrido[3,2-e][1,2,4]triazolo[4,3-c]pyrimidin-3(2H)-one When performing the Suzuki coupling to prepare the product of Example 18, the title compound was isolated as a side product. HPLC/MS: 508.2 (M+1), 510.2 (M+3); Rₜ=3.81 min.

### Example 28

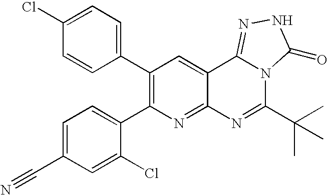

4-[5-Tert-butyl-9-(4-chlorophenyl)-3-oxo-2,3-dihydropyrido[3,2-e][1,2,4]triazolo[4,3-c]pyrimidin-8-yl]-3-chlorobenzonitrile. To the product of Example 3 (250 mg, 0.460 mmol) was added 18-crown-6 (182 mg, 0.690 mmol), potassium cyanide (69.0 mg, 1.06 mmol), tetrakis(triphenylphosphine)palladium (0) (213 mg, 0.184 mmol) and 1,4-dioxane (5 mL). The flask was purged with N₂ for 5 minutes before heating to 95° C. for 1 h. The reaction was diluted with EtOAc, washed with brine and concentrated. The residue was purified by flash chromatography on silica gel gradient eluted with 0-35% EtOAc in hexane affording the title compound. HPLC/MS: 489.0 (M+1), 491.0 (M+3); Rₜ=3.68 min.

The following examples were prepared in similar fashion to Example 28 except using sodium cyanide in place of potassium cyanide.

| | | HPLC/MS | |
|---|---|---|---|
| | | m/z (M + 1) | |
| | | m/z (M + 3) | |
| Example | Name | Rₜ (min) | Structure |
| Example 29 | 2-[5-Tert-butyl-9-(4-chlorophenyl)-3- oxo-2,3-dihydropyrido[3,2- e][1,2,4]triazolo[4,3-c]pyrimidin-8-yl]- 5-chlorobenzonitrile | 489.0 491.0 3.70 | 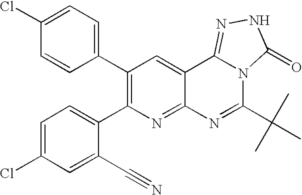 |
| Example 30 | 2-[5-Tert-butyl-9-(4-chlorophenyl)-3- oxo-2,3-dihydropyrido[3,2- e][1,2,4]triazolo[4,3-c]pyrimidin-8- yl]benzonitrile | 455.2 457.1 3.49 | 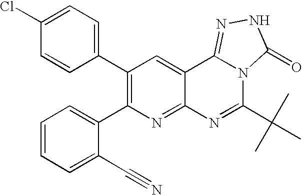 |

### Example 31

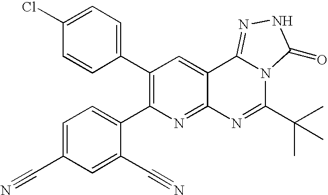

4-[5-Tert-butyl-9-(4-chlorophenyl)-3-oxo-2,3-dihydropyrido[3,2-e][1,2,4]triazolo[4,3-c]pyrimidin-8-yl]isophthalonitrile. The title compound was isolated as a side product from Example 28. HPLC/MS: 480.1 (M+1), 482.0 (M+3); Rₜ=3.46 min.

### Example 32

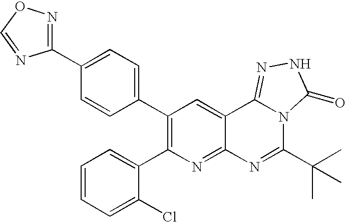

### 5-Tert-butyl-8-(2-chlorophenyl)-9-[4-(1,2,4-oxadiazol-3-yl)phenyl]pyrido[3,2-e][1,2,4]-triazolo[4,3-c]pyrimidin-3(2H)-one

Step A: 2-Tert-butyl-7-(2-chlorophenyl)-6-[4-(1,2,4-oxadiazol-3-yl)phenyl]pyrido[2,3-d]pyrimidin-4(3H)-one. To 4-[2-tert-butyl-7-(2-chlorophenyl)-4-oxo-3,4-dihydropyrido[2,3-d]pyrimidin-6-yl]benzonitrile (210 mg, 0.506 mmol, prepared in similar fashion to compounds in the preceding examples) was added hydroxylamine hydrochloride (184 mg, 2.65 mmol), ethanol (8 mL), dioxane (3 mL) and NEt₃ (0.706 mL, 5.06 mmol). The reaction was heated to 85° C. for about 6 h and concentrated. The residue was diluted with xylenes (5 mL) and triethylorthoformate (1 mL), heated to about 120° C. for 40 min and concentrated. The residue was purified by flash chromatography on silica gel gradient eluted with 0-100% EtOAc in hexane affording the title compound. HPLC/MS: 458.1 (M+1), 460.1 (M+3); Rₜ=3.37 min.

Step B: 5-Tert-butyl-8-(2-chlorophenyl)-9-[4-(1,2,4-oxadiazol-3-yl)phenyl]pyrido[3,2-e][1,2,4]triazolo[4,3-c]pyrimidin-3(2H)-one. Using the product of Step A and the general procedure of Example I Steps F-H the title compound was prepared. HPLC/MS: 498.1 (M+1), 500.1 (M+3); Rₜ=3.52 min.

### Example 33

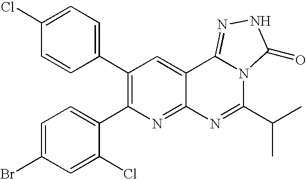

8-(4-Bromo-2-chlorophenyl)-9-(4-chlorophenyl)-5-isopropylpyrido[3,2-e][1,2,4]triazolo[4,3-c]pyrimidin-3(2H)-one. Using the appropriate starting materials and following the general procedure of Example 1 the title compound was obtained. HPLC/MS: 528.0 (M+1), 530.0 (M+3); Rₜ=3.86 min.

### Example 34

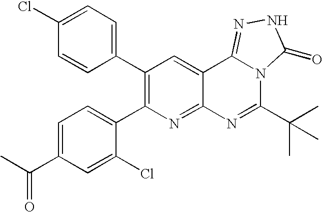

8-(4-Acetyl-2-chlorophenyl)-5-tert-butyl-9-(4-chlorophenyl)pyrido[3,2-e][1,2,4]triazolo[4,3-c]pyrimidin-3(2H)-one. To the product of Example 28 (92.7 mg, 0.189 mmol) in THF (2 mL) was added methyllithium (1.6 M in ether, 0.3 mL, 0.480 mmol) at −45° C. The reaction was warmed to −4° C., but HPLC/MS indicated incomplete reaction. The reaction was cooled to −45° C. before adding methyllithium (1.6 M in ether, 0.1 mL, 0.160 mmol). The reaction was then quenched with saturated aq NH₄Cl, diluted with EtOAc, washed with brine and concentrated. The residue was purified by flash chromatography on silica gel gradient eluted with 0-20% EtOAc in CH₂Cl₂ affording the title compound. HPLC/MS: 506.1 (M+1), 508.1 (M+3); Rₜ=3.63 min.

### Biological Example 1

### Cannabinoid Receptor-1 (CB1) Binding Assay

Binding affinity determination is based on recombinant human CB1 receptor expressed in Chinese Hamster Ovary (CHO) cells (Felder et al, Mol. Pharmacol. 48: 443-450, 1995). Total assay volume is 250 μl (240 μl CB1 receptor membrane solution plus 5 μl test compound solution plus 5 μl [3H]CP-55940 solution). Final concentration of [3H]CP-55940 is 0.6 nM. Binding buffer contains 50 mM Tris-HCl, pH7.4, 2.5 mM EDTA, 5 mM MgCl₂, 0.5 mg/mL fatty acid free bovine serum albumin and protease inhibitors (Cat#P8340, from Sigma). To initiate the binding reaction, 5 μl of radioligand solution is added, the mixture is incubated with gentle shaking on a shaker for 1.5 h at 30° C. The binding is terminated by using 96-well harvester and filtering through GF/C filter presoaked in 0.05% polyethylenimine. The bound radiolabel is quantitated using scintillation counter. Apparent binding affinities for various compounds are calculated from IC₅₀ values (DeBlasi et al., Trends Pharmacol Sci 10: 227-229, 1989). Compounds of the present invention have have IC50s of less than 5 micromolar in the CB1 binding assay. In particular, compounds of Examples 1 to 34 were assayed in the CB1 Binding assay and found to have IC₅₀ values for the human CB1 receptor of less than 1.2 micromolar.

The binding assay for CB2 receptor is done similarly with recombinant human CB2 receptor expressed in CHO cells. The compounds of the present invention are selective CB1 antagonist/inverse agonist compounds having IC50s greater in the CB2 binding assay than in the CB1 assay.

CB1 Receptor Binding Activity for Selected Compounds

| | CB1 binding |
|---|---|
| Example No. | IC₅₀ (nM) |
| 1 | 2 |
| 9 | 10 |
| 16 | 6 |
| 27 | 6 |
| 28 | 1 |
| 31 | 4 |
| 32 | 11 |

### Biological Example 2

### Cannabinoid Receptor-1 (CB1) Functional Activity Assay

The functional activation of CB1 receptor is based on recombinant human CB1 receptor expressed in CHO cells (Felder et al, Mol. Pharmacol. 48: 443-450, 1995). To determine the agonist activity or inverse agonist activity of any test compound, 50 ul of CB1-CHO cell suspension are mixed with test compound and 70 ul assay buffer containing 0.34 mM 3-isobutyl-1-methylxanthine and 5.1 uM of forskolin in 96-well plates. The assay buffer is comprised of Earle's Balanced Salt Solution supplemented with 5 mM MgCl₂, 1 mM glutamine, 10 mM HEPES, and 1 mg/mL bovine serum albumin. The mixture is incubated at room temperature for 30 minutes, and terminated by adding 30 ul/well of 0.5M HCl. The total intracellular cAMP level is quantitated using the New England Nuclear Flashplate and cAMP radioimmunoassay kit.

The compounds of Examples 1, 9, 16, 27, 28, 31, and 32 were all tested in the CB1 functional activity assay and found to have EC50s less than 100 nM.

### Biological Example 3

### Cannabinoid Receptor-1 (CB1) Functional Antagonist Assay

To determine the antagonist activity of test compound, the reaction mixture also contains 0.5 nM of the agonist CP55940 (or 50 nM of methanandamide), and the reversal of the CP55940 (or methanandamide) effect is quantitated with increasing concentration of the test compound. Intracellular cAMP is determined as described above. An IC50 value for the test compound is calculated from the titration curve.

Alternatively, a series of dose response curves for the agonist CP55940 (or methanandamide) is performed with increasing concentration of the test compound in each of the dose response curves, and a Schild analysis is carried to calculate the Kb value which is an estimation of test compound binding affinity.

The compounds of Examples 1, 9, 16, 27, 28, 31, and 32 were all tested in the CB1 functional activity assay and were functional inverse agonists.

### Biological Example 4

### Cannabinoid Receptor-2 (CB2) Functional Activity Assay

The functional assay for the CB2 receptor is done similarly with recombinant human CB2 receptor expressed in CHO cells.

### Biological Example 5

### Acute Food Intake Studies in Rats or Mice: General Procedure

Adult rats or mice are used in these studies. After at least 2 days of acclimation to the vivarium conditions (controlled humidity and temperature, lights on for 12 hours out of 24 hours) food is removed from rodent cages. Experimental compounds or their vehicles are administered orally, intraperitoneally, subcutaneously or intravenously before the return of a known amount of food to cage. The optimal interval between dosing and food presentation is based on the half-life of the compound based on when brain concentrations of the compound is the highest. Food remaining is measured at several intervals. Food intake is calculated as grams of food eaten per gram of body weight within each time interval and the appetite-suppressant effect of the compounds are compared to the effect of vehicle. In these experiments many strains of mouse or rat, and several standard rodent chows can be used.

### Biological Example 6

### Chronic Weight Reduction Studies in Rats or Mice: General Procedure

Adult rats or mice are used in these studies. Upon or soon after weaning, rats or mice are made obese due to exclusive access to diets containing fat and sucrose in higher proportions than in the control diet. The rat strains commonly used include the Sprague Dawley bred through Charles River Laboratories. Although several mouse strains may be used, c57Bl/6 mice are more prone to obesity and hyperinsulinemia than other strains. Common diets used to induce obesity include: Research Diets D12266B (32% fat) or D12451 (45% fat) and BioServ S3282 (60% fat). The rodents ingest chow until they are significantly heavier and have a higher proportion of body fat than control diet rats, often 9 weeks. The rodents receive injections (1 to 4 per day) or continuous infusions of experimental compounds or their vehicles either orally, intraperitoneally, subcutaneously or intravenously. Food intake and body weights are measured daily or more frequently. Food intake is calculated as grams of food eaten per gram of body weight within each time interval and the appetite-suppressant and weight loss effects of the compounds are compared to the effects of vehicle.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A compound of structural formula I: 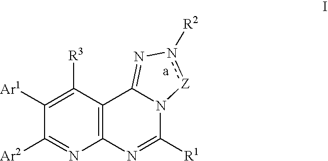
or a pharmaceutically acceptable salt thereof, wherein:
Z is selected from:
(1) C═O, and
(2) CR⁸;
“a” is:
(1) a single bond when R² is present and Z is C═O, or
(2) a double bond when R² is absent and Z is CR⁸;
Ar¹ is selected from:
(1) aryl, and
(2) heteroaryl,
wherein aryl and heteroaryl are unsubstituted or substituted with one, two, three or four substituents selected from R⁴ and R⁵;
Ar² is aryl, wherein aryl is unsubstituted or substituted with one, two, three or four substituents independently selected from R⁶ and R⁷;
R¹ is selected from:
(1) C₁₋₁₀alkyl,
(2) C₃₋₁₀cycloalkyl,
(3) C₃₋₁₀cycloalkenyl,
(4) C₃₋₁₀cycloalkyl-C₁₋₄alkyl,
(5) C₃₋₁₀cycloalkenyl-C₁₋₄alkyl,
(6) cycloheteroalkyl,
(7) cycloheteroalkyl-C₁₋₄alkyl,
(8) aryl,
(9) aryl-C₁₋₄alkyl,
(10) heteroaryl,
(11) heteroaryl-C₁₋₄alkyl,
(12) OR^{e}, and
(13) NR^{c}R^{d},
wherein each alkyl is unsubstituted or substituted with one to four substituents independently selected from R^{a}, and each cycloalkyl, cycloalkenyl, cycloheteroalkyl, aryl and heteroaryl is unsubstituted or substituted with one to four substituents independently selected from R^{b};
R² is absent or selected from:
(1) hydrogen, and
(2) C₁₋₁₀alkyl;
R³ is hydrogen;
each R⁴, R⁵, R⁶, and R⁷ is independently selected from:
(1) -hydrogen,
(2) -halogen,
(3) —CN,
(4) —C₁₋₆alkyl unsubstituted or substituted with one, two or three R^{f} substitutents,
(5) —CF₃,
(6) —C₂₋₆alkenyl, unsubstituted or substituted with one, two or three R^{f} substitutents,
(7) -cycloalkyl, unsubstituted or substituted with one, two or three R^{f} substitutents,
(8) -cycloalkyl-C₁₋₃alkyl-, unsubstituted or substituted with one, two or three R^{f} substitutents,
(9) -cycloheteroalkyl, unsubstituted or substituted with one, two or three R^{f} substitutents,
(10) -aryl, unsubstituted or substituted with one, two or three R^{h} substitutents,
(11) -aryl-C₁₋₃alkyl-, unsubstituted or substituted on aryl with one, two or three R^{h} substitutents,
(12) -heteroaryl, unsubstituted or substituted with one, two or three R^{h} substitutents,
(13) -heteroaryl-C₁₋₃alkyl-, unsubstituted or substituted with one, two or three R^{h} substitutents,
(14) —OR^{d},
(15) —OCF₃,
(16) —C(O)R^{d},
(17) —S(O)ₘC₁₋₆alkyl, and
(18) —NR^{c}R^{d};
R⁸ is selected from:
(1) hydrogen,
(2) halogen,
(3) C₁₋₁₀alkyl,
(4) (CH₂)ₙ—O—C₁₋₆alkyl,
(5) (CH₂)ₙ—NH₂,
(6) (CH₂)ₙ—NH(C₁₋₆alkyl), and
(7) (CH₂)ₙ—N(C₁₋₆alkyl)₂,
wherein each alkyl is unsubstituted or substituted with one to four substituents independently selected from R^{a};
each R^{a} is independently selected from:
(1) —OR^{d},
(2) —NR^{c}S(O)ₘR^{d},
(3) halogen,
(4) —SR^{d},
(5) —S(O)ₘNR^{c}R^{d},
(6) —NR^{c}R^{d},
(7) —C(O)R^{d},
(8) —CO₂R^{d},
(9) —CN,
(10) —C(O)NR^{c}R^{d},
(11) —NR^{c}C(O)OR^{d},
(12) —NR^{c}C(O)OR^{d},
(13) —NR^{c}C(O)NR^{c}R^{d},
(14) —CF₃, and
(15) —OCF₃;
each R^{b} is independently selected from:
(1) R^{a},
(2) oxo,
(3) Cl₁₋₁₀alkyl,
(4) C₂₋₁₀alkenyl,
(5) cycloalkyl,
(6) cycloalkyl-C₁₋₁₀alkyl,
(7) cycloheteroalkyl,
(8) cycloheteroalkyl-C₁₋₁₀ alkyl,
(9) aryl,
(10) heteroaryl,
(11) aryl-C₁₋₁₀alkyl, and
(12) heteroaryl-C₁₋₁₀alkyl,
wherein alkyl and alkenyl are unsubstituted or substituted with one, two, three or four R^{h} substituents, and cycloalkyl, cycloheteroalkyl, aryl and heteroaryl are unsubstituted or substituted with one, two or three R^{h} substituents;
R^{c}C and R^{d} are each independently selected from:
(1) hydrogen,
(2) C₁₋₁₀alkyl,
(3) C₂₋₁₀ alkenyl,
(4) cycloalkyl,
(5) cycloalkyl-C₁₋₁₀alkyl-,
(6) cycloheteroalkyl,
(7) cycloheteroalkyl-C₁₋₁₀alkyl-,
(8) aryl,
(9) heteroaryl,
(10) aryl-C₁₋₁₀alkyl-, and
(11) heteroaryl-C₁₋₁₀alkyl-,
wherein alkyl, alkenyl, cycloalkyl, cycloheteroalkyl, aryl and heteroaryl are unsubstituted or substituted with one to three substituents selected from R^{f};
each R^{e} is independently selected from:
(1) C₁₋₁₀alkyl,
(2) aryl,
(3) heteroaryl,
(4) cycloalkyl, and
(5) cycloheteroalkyl;
wherein alkyl, cycloalkyl, cycloheteroalkyl, aryl and heteroaryl are unsubstituted or substituted with one, two, or three substituents independently selected from R^{h};
each R^{f} is independently selected from:
(1) halogen,
(2) C₁₋₆alkyl,
(3) 4-methylbenzyl-,
(4) —OH,
(5) —O—C₁₋₄alkyl,
(6) benzyloxy-,
(7) -oxo,
(8) —OC(O)—C₁₋₆alkyl,
(9) —C(O)O—C₁₋₆alkyl,
(10) —S—C₁₋₄alkyl,
(11) —NH(CH₃),
(12) —N(CH₃)₂,
(13) —NO₂,
(14) —CN,
(15) —CF₃, and
(16) —OCF₃,
wherein alkyl may be unsubstituted or substituted with one, two or three substituents selected from R^{g};
each R^{g} is independently selected from:
(1) halogen,
(2) —O—C₁₋₄alkyl,
(3) —OH,
(4) —S—C₁₋₄alkyl,
(5) —CN,
(6) —CF₃, and
(7) —OCF₃;
each R^{h} is independently selected from:
(1) halogen,
(2) oxo,
(3) amino,
(4) hydroxy,
(5) C₁₋₄alkyl,
(6) —O—C₁₋₄alkyl,
(7) —S—C₁₋₄alkyl,
(8) —CN,
(9) —CF₃, and
(10) —OCF₃;
each m is independently selected from 0, 1 and 2; and
each n is independently selected from 0, 1, 2 and 3.

2. The compound according to claim 1, wherein Ar² is phenyl, wherein phenyl is substituted with R⁶ and R⁷, or a pharmaceutically acceptable salt thereof.

3. The compound according to claim 1, wherein Ar¹ is selected from phenyl and pyridyl, wherein phenyl and pyridyl are substituted with R⁴ and R⁵, or a pharmaceutically acceptable salt thereof.

4. The compound according to claim 1, wherein R¹ is C₁₋₁₀alkyl, wherein alkyl is unsubstituted or substituted with one to four substituents independently selected from R^{a}, or a pharmaceutically acceptable salt thereof.

5. The compound according to claim 1, wherein each R⁴, R⁵, R⁶, and R⁷ is independently selected from:
(1) -hydrogen,
(2) -halogen,
(3) —CN,
(4) —C₁₋₆alkyl, unsubstituted or substituted with one, two or three R^{f} substitutents,
(5) —CF₃,
(6) -heteroaryl, unsubstituted or substituted with one, two or three R^{h} substitutents,
(7) —OR^{d},
(8) —OCF₃,
(9) —C(O)R^{d},
(10) —S(O)ₘC₁₋₆alkyl, and
(11) NR^{c}R^{d},
or a pharmaceutically acceptable salt thereof.

6. The compound according to claim 1, wherein “a” is a single bond, R² is hydrogen, and Z is C═O, or a pharmaceutically acceptable salt thereof.

7. The compound according to claim 1, wherein “a” is a double bond, R² is absent, and Z is CH, or a pharmaceutically acceptable salt thereof.

8. The compound according to claim 1 of structural formula ID: 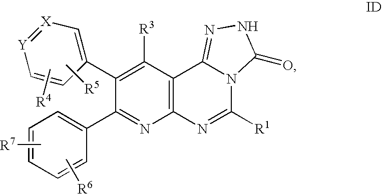
or a pharmaceutically acceptable salt thereof, wherein:
X is CH or N;
Y is CH or N, provided that at least one of X and Y is CH; and
R¹, R³, R⁴, R⁵, R⁶ and R⁷ are as defined in claim 1.

9. The compound according to claim 1 of structural formula IE: 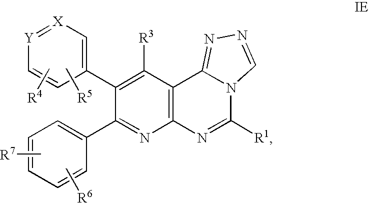
or a pharmaceutically acceptable salt thereof, wherein:
X is CH or N;
Y is CH or N, provided that at least one of X and Y is CH; and
R¹, R³, R⁴, R⁵, R⁶ and R⁷ are as defined in claim 1.

10. The compound according to claim 1, selected from:
(1) 5-tert-butyl-8-(2-chlorophenyl)-9-(4-chlorophenyl)pyrido[3,2-e][1,2,4]triazolo[4,3-c]pyrimidin-3(2H)-one;
(2) 9-bromo-5-tert-butyl-8-(2-chlorophenyl)pyrido[3,2-e][1,2,4]triazolo[4,3-c]pyrimidin-3(2H)-one;
(3) 8-(4-bromo-2-chlorophenyl)-5-tert-butyl-9-(4-chlorophenyl)pyrido[3,2-e][1,2,4]triazolo[4,3-c]pyrimidin-3(2H)-one;
(4) 5-tert-butyl-9-(4-chlorophenyl)-8-(2-methylphenyl)pyrido[3,2-e][1,2,4]triazolo[4,3-c]pyrimidin-3(2H)-one;
(5) 9-(4-bromophenyl)-5-tert-butyl-8-(2-chlorophenyl)pyrido[3,2-e][1,2,4]triazolo[4,3-c]pyrimidin-3(2H)-one;
(6) 8-(2-bromophenyl)-5-tert-butyl-9-(4-chlorophenyl)pyrido[3,2-e][1,2,4]triazolo[4,3-c]pyrimidin-3 (2H)-one;
(7) 8-(2-bromo-4-chlorophenyl)-5-tert-butyl-9-(4-chlorophenyl)pyrido[3,2-e][1,2,4]triazolo[4,3-c]pyrimidin-3 (2H)-one;
(8) 5-tert-butyl-8-(2-chloro-3-methylphenyl)-9-(4-chlorophenyl)pyrido[3,2-e][1,2,4]triazolo[4,3-c]pyrimidin-3 (2H)-one;
(9) 9-(4-acetylphenyl)-5-tert-butyl-8-(2-chlorophenyl)pyrido[3,2-e][1,2,4]triazolo[4,3-c]pyrimidin-3(2H)-one;
(10) 5-tert-butyl-8-(2-chlorophenyl)-9-(3-chlorophenyl)pyrido[3,2-e][1,2,4]triazolo[4,3-c]pyrimidin-3 (2H)-one;
(11) 5-tert-butyl-8-(2-chlorophenyl)-9-(4-methylphenyl)pyrido[3,2-e][1,2,4]triazolo[4,3-c]pyrimidin-3 (2H)-one;
(12) 5-tert-butyl-8-(2-chlorophenyl)-9-[4-(dimethylamino)phenyl]pyrido[3,2-e][1,2,4]triazolo[4,3-c]pyrimidin-3(2H)-one;
(13) 5-tert-butyl-8-(2-chlorophenyl)-9-[4-(trifluoromethoxy)phenyl]pyrido[3,2-e][1,2,4]triazolo[4,3-c]pyrimidin-3(2H)-one;
(14) 5-tert-butyl-8-(2-chlorophenyl)-9-(4-methoxyphenyl)pyrido[3,2-e][1,2,4]triazolo[4,3-c]pyrimidin-3 (2H)-one;
(15) 5-tert-butyl-8-(2-chlorophenyl)-9-(4-isopropoxyphenyl)pyrido[3,2-e][1,2,4]triazolo[4,3-c]pyrimidin-3(2H)-one;
(16) 4-[5-tert-butyl-8-(2-chlorophenyl)-3-oxo-2,3-dihydropyrido[3,2-e][1,2,4]triazolo[4,3-c]pyrimidin-9-yl]benzonitrile;
(17) 5-tert-butyl-8-(2-chlorophenyl)-9-[4-(trifluoromethyl)phenyl]pyrido[3,2-e][1,2,4]triazolo[4,3-c]pyrimidin-3(2H)-one;
(18) 5-tert-butyl-8-(2-chlorophenyl)-9-(4-fluorophenyl)pyrido[3,2-e][1,2,4]triazolo[4,3-c]pyrimidin-3(2H)-one;
(19) 5-tert-butyl-8-(2-chlorophenyl)-9-(3,4-difluorophenyl)pyrido[3,2-e][1,2,4]triazolo[4,3-c]pyrimidin-3 (2H)-one;
(20) 5-tert-butyl-8-(2-chlorophenyl)-9-(3,5-difluorophenyl)pyrido[3,2-e][1,2,4]triazolo[4,3-c]pyrimidin-3(2H)-one;
(21) 5-tert-butyl-9-(4-chloro-3-methylphenyl)-8-(2-chlorophenyl)pyrido[3,2-e][1,2,4]triazolo[4,3-c]pyrimidin-3(2H)-one;
(22) 5-tert-butyl-8-(2-chlorophenyl)-9-[4-(methylsulfonyl)phenyl]pyrido[3,2-e][1,2,4]triazolo[4,3-c]pyrimidin-3(2H)-one;
(23) 5-tert-butyl-8-(2-chlorophenyl)-9-pyridin-4-ylpyrido[3,2-e][1,2,4]triazolo[4,3-c]pyrimidin-3(2H)-one;
(24) 5-tert-butyl-8-(2-chlorophenyl)-9-pyrimidin-5-ylpyrido[3,2-e][1,2,4]triazolo[4,3-c]pyrimidin-3(2H)-one;
(25) 5-tert-butyl-8-(2-chlorophenyl)-9-pyridin-3-ylpyrido[3,2-e][1,2,4]triazolo[4,3-c]pyrimidin-3(2H)-one;
(26) 5-tert-butyl-8-(2-chlorophenyl)-9-(6-fluoropyridin-3-yl)pyrido[3,2-e][1,2,4]triazolo[4,3-c]pyrimidin-3(2H)-one;
(27) 5-[5-tert-butyl-8-(2-chlorophenyl)-3-oxo-2,3-dihydropyrido[3,2-e][1,2,4]triazolo[4,3-c]pyrimidin-9-yl]pyridine-2-carbonitrile;
(28) 5-tert-butyl-8-(4′-fluorobiphenyl-2-yl)-9-(4-fluorophenyl)pyrido[3,2-e][1,2,4]triazolo[4,3-c]pyrimidin-3(2H)-one;
(29) 4-[5-tert-butyl-9-(4-chlorophenyl)-3-oxo-2,3-dihydropyrido[3,2-e][1,2,4]triazolo[4,3-c]pyrimidin-8-yl]-3-chlorobenzonitrile];
(30) 2-[5-tert-butyl-9-(4-chlorophenyl)-3-oxo-2,3-dihydropyrido[3,2-e][1,2,4]triazolo[4,3-c]pyrimidin-8-yl]-5-chlorobenzonitrile;
(31) 2-[5-tert-butyl-9-(4-chlorophenyl)-3-oxo-2,3-dihydropyrido[3,2-e][1,2,4]triazolo[4,3-c]pyrimidin-8-yl]benzonitrile;
(32) 4-[5-tert-butyl-9-(4-chlorophenyl)-3-oxo-2,3-dihydropyrido[3,2-e][1,2,4]triazolo[4,3-c]pyrimidin-8-yl]isophthalonitrile;
(33) 5-tert-butyl-8-(2-chlorophenyl)-9-[4-(1,2,4-oxadiazol-3-yl)phenyl]pyrido[3,2-e][1,2,4]triazolo[4,3-c]pyrimidin-3(2H)-one;
(34) 8-(4-bromo-2-chlorophenyl)-9-(4-chlorophenyl)-5-isopropylpyrido[3,2-e][1,2,4]triazolo[4,3-c]pyrimidin-3(2H)-one;
(35) 8-(4-acetyl-2-chlorophenyl)-5-tert-butyl-9-(4-chlorophenyl)pyrido[3,2-e][1,2,4]triazolo[4,3-c]pyrimidin-3(2H)-one; and
(36) s-tert-butyl-8-(2-chlorophenyl)-9-(4-chlorophenyl)pyrido[3,2-e][1,2,4]triazolo[4,3-c]pyrimidine;
or a pharmaceutically acceptable salt thereof.

11. A composition comprising a compound according to claim 1 and a pharmaceutically acceptable carrier.

12. A composition comprising a compound according to claim 10 and a pharmaceutically acceptable carrier.

13-16. (canceled)

17. A method of treating a condition ameliorated by antagonism or inverse agonism of the CB1 receptor in a patient in need thereof comprising administration of a therapeutically effective amount of a compound according to claim 1.

18. A method of preventing obesity in a patient at risk for obesity comprising administration of about 0.01 mg to about 50 mg of a compound according to claim 1.

19. The method of claim 17 wherein the condition is selected from: psychosis, memory deficit, cognitive disorders, Alzheimer's disease, migraine, neuropathy, neuro-inflammatory disorders, cerebral vascular accidents, head trauma, anxiety disorders, stress, epilepsy, Parkinson's disease, schizophrenia, substance abuse disorders, constipation, chronic intestinal pseudo-obstruction, cirrhosis of the liver, asthma, obesity, and other eating disorders associated with excessive food intake.

20. The method of claim 19, wherein the substance abuse disorder is abuse of or addiction to a substance selected from: opiates, alcohol, marijuana, and nicotine, and the eating disorder associated with excessive food intake is selected from obesity, bulimia nervosa, and compulsive eating disorders.
